(19) United States
(12) Patent Application Publication
(10) Pub. No.: US 2023/0348843 A1
(43) Pub. Date: Nov. 2, 2023

(54) METHODS FOR CONTROLLING GEOMETRIC REGULARITY AND HOMOGENEITY OF AERIAL MYCELIUM TOPOLOGIES AND PRODUCTS OF AERIAL MYCELIUM WITH GEOMETRICALLY REGULAR OR HOMOGENEOUS TOPOLOGIES

(71) Applicant: Ecovative Design LLC, Green Island, NY (US)

(72) Inventors: Asa Trench Snyder, Petersburg, NY (US); Jacob Michael Winiski, Troy, NY (US); Eben D. Bayer, Troy, NY (US)

(21) Appl. No.: 18/181,497

(22) Filed: Mar. 9, 2023

Related U.S. Application Data
(60) Provisional application No. 63/421,087, filed on Oct. 31, 2022.
(60) Provisional application No. 63/318,653, filed on Mar. 10, 2022.

(51) Int. Cl. C12N 1/14; A23L 31/00

(52) U.S. Cl. CPC C12N 1/14; A23L 31/00; C12N 2513/00

(57) ABSTRACT

This application relates to a method for growing aerial mycelium with a regular growth pattern and products of aerial mycelium biopolymers with regular growth patterns. For example, a regular growth pattern can include a homogeneous growth topology or a geometrically regular pattern of bulbous forms. As further examples, the method for affecting growth topology can include controlling environmental conditions or using a topology adjustment layer.

### CROSS-REFERENCE TO RELATED APPLICATIONS

This application is related to U.S. Provisional Patent Application No. 63/318,653, filed Mar. 10, 2022, entitled “METHODS FOR CONTROLLING GEOMETRIC REGULARITY AND HOMOGENEITY OF AERIAL MYCELIUM TOPOLOGIES AND PRODUCTS OF AERIAL MYCELIUM WITH GEOMETRICALLY REGULAR OR HOMOGENEOUS TOPOLOGIES,” and U.S. Provisional Patent Application No. 63/421,087, filed Oct. 31, 2022, entitled “METHODS FOR CONTROLLING GEOMETRIC REGULARITY AND HOMOGENEITY OF AERIAL MYCELIUM TOPOLOGIES AND PRODUCTS OF AERIAL MYCELIUM WITH GEOMETRICALLY REGULAR OR HOMOGENEOUS TOPOLOGIES,” the disclosures of which are incorporated herein by reference in their entirety to the extent not inconsistent with the content of this disclosure.

### INCORPORATION BY REFERENCE TO ANY PRIORITY APPLICATIONS

Any and all applications for which a foreign or domestic priority claims is identified in the Application Data Sheet as filed with the present application are hereby incorporated by reference under 37 C.F.C. § 1.57. This application claims the benefit of U.S. Provisional Patent Application No. 63/318,653, filed Mar. 10, 2022, entitled “METHODS FOR CONTROLLING GEOMETRIC REGULARITY AND HOMOGENEITY OF AERIAL MYCELIUM TOPOLOGIES AND PRODUCTS OF AERIAL MYCELIUM WITH GEOMETRICALLY REGULAR OR HOMOGENEOUS TOPOLOGIES,” and U.S. Provisional Patent Application No. 63/421,087, filed Oct. 31, 2022, entitled “METHODS FOR CONTROLLING GEOMETRIC REGULARITY AND HOMOGENEITY OF AERIAL MYCELIUM TOPOLOGIES AND PRODUCTS OF AERIAL MYCELIUM WITH GEOMETRICALLY REGULAR OR HOMOGENEOUS TOPOLOGIES,” the disclosure of which is incorporated herein by reference in its entirety.

### BACKGROUND

### Field

This application relates generally to improved aerial mycelium products and methods for growing aerial mycelium, and in particular, for aerial mycelium with improved growth surface topologies.

### Description

Aerial mycelium can be implemented for use in the manufacture of various products, such as mycelium-based textile products, leather-like materials, petroleum-based product alternatives and foams, and various food products, such as meat substitutes. As a result of the growth process, aerial mycelium can include varying degrees of topological growth. There is a need for aerial mycelium products and methods that include desired topological features.

### SUMMARY

For purposes of summarizing the invention and the advantages achieved over the prior art, certain objects and advantages of the invention have been described herein. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

In a first aspect, a method of growing an aerial mycelium biopolymer is described. The method can include providing an inoculated substrate comprising a first substrate and a first spawn interspersed throughout the first substrate. The first spawn can include a second substrate for fungal growth and a first fungus. The first spawn can also include a solid fungal inoculum. The method can further include providing a second spawn comprising a third substrate for fungal growth and a second fungus and positioning the second spawn in a growth pattern approximately parallel to an outer surface of the inoculated substrate, wherein the growth pattern is configured to affect a growth topology of aerial mycelium growing from the first spawn and the second spawn.

In various aspects, the growth pattern can be configured to increase the homogeneity of the growth topology.

In some aspects, the method can also include forming a layer of uninoculated substrate on at least one of the inoculated substrate and the second spawn, wherein the uninoculated substrate comprises a fourth substrate without substantially any fungus. The step of forming the layer of uninoculated substrate can include forming a first layer of uninoculated substrate on the inoculated substrate and forming a second layer of uninoculated substrate on the second spawn.

In another aspect, the step of positioning can include positioning the second spawn directly onto the outer surface of the inoculated substrate.

In various aspects, the method can also include providing a first topology adjustment layer, wherein the first topology adjustment layer comprises one or more openings.

The step of positioning can include positioning the first topology adjustment layer adjacent to the outer surface of the inoculated substrate wherein the one or more openings of the first topology adjustment layer form the growth pattern and spreading the second spawn across the one or more openings of the first topology adjustment layer. In some aspects, the step of positioning can also include removing the first topology adjustment layer.

In various aspects, the one or more openings of the first topology adjustment layer can include a plurality of holes in a grid pattern across the first topology adjustment layer.

In some aspects, the method can include providing a second topology adjustment layer.

In some aspects, the step of positioning can also include positioning the first or second topology adjustment layer adjacent to the outer surface of the inoculated substrate, wherein one or more of the one or more openings of the first or second topology adjustment layer comprise perforations, and wherein the first or second topology adjustment layer is configured to allow for growth through the perforations and further affect a growth topology of aerial mycelium growing from the first spawn and the second spawn.

In various aspects, the method can include placing the inoculated substrate and the second spawn into an incubation chamber; maintaining the incubation chamber with a predetermined growth environment of humidity, temperature, carbon dioxide content, and oxygen content sufficient to produce an aerial mycelium biopolymer consisting essentially of fungal mycelium; and incubating the inoculated substrate and the second spawn in the incubation chamber for a period of time sufficient to produce the aerial mycelium biopolymer.

In some aspects, the step of maintaining the incubation chamber can include maintaining the carbon dioxide content within the incubation chamber at a level between about 0.6% to about 7% to affect the vertical growth topology of aerial mycelium. The method of maintaining the incubation chamber can also include maintaining the humidity by introducing aqueous mist into the incubation chamber at a rate of about 0.01 mg/cm²/hr to about 1 mg/cm²/hr to affect the vertical growth topology of aerial mycelium.

In some aspects, the method can include growing the aerial mycelium through the one or more openings of the first or second topology adjustment layer.

In various aspects, the method can also include peeling the first or second topology adjustment layer to separate the aerial mycelium biopolymer from the inoculated substrate.

In another aspect, a method of growing an aerial mycelium biopolymer is described. The method can include providing an inoculated substrate comprising a first substrate and a spawn interspersed throughout the substrate, wherein the spawn comprises a second substrate for fungal growth and a first fungus. The method can further include treating the inoculated substrate to prevent growth of the spawn within an inviable portion and permit growth of the remaining spawn within a viable portion, wherein the viable portion forms a growth pattern configured to affect the growth topology of aerial mycelium growing from the inoculated substrate.

In another aspect, an aerial mycelium biopolymer obtained from any one of the preceding methods is described.

In various aspects, the aerial mycelium biopolymer can include a growth surface, wherein the growth surface has a mean native height, and wherein the coefficient of variation of the mean native height is less than 2. In some aspects, the growth surface can include a plurality of bulbs forming a preselected pattern. In some further aspects, the plurality of bulbs can be regularly-spaced. In some aspects, the growth surface can include a plurality of depressions forming a preselected pattern.

In some aspects, the aerial mycelium biopolymer panel can include a morphological segmentation of each of a plurality of point clouds based on their Z by XY luminance height profiles, comprising a segment to area ratio of less than 1 segment per square inch.

In some aspects, the aerial mycelium biopolymer panel can include a morphological segmentation of each of a plurality of point clouds based on their Z by XY luminance height profiles, comprising a mean segment area of greater than 875 mm².

In various aspects, the aerial mycelium biopolymer panel can have a displacement at tensile strength of at least 40% and a density of between about 1.5 lb/ft³ and about 10.5 lb/ft³.

In some aspects, the arial mycelium biopolymer panel can include a growth surface, wherein the growth surface includes at least one of a plurality of bulbs and a plurality of depressions, wherein the at least one of the plurality of bulbs and the plurality of depressions form a preselected pattern. In some aspects, the growth surface can include a plurality of bulbs, wherein the plurality of bulbs can be regularly-spaced. In some further aspects, the growth surface can include a plurality of regularly-spaced depressions forming a preselected pattern.

In another aspect, a method for inducing a homogeneity of surface topology in a mycelium growth surface is described. The method can include providing a substrate on a tool, placing at least one fungus for aerial mycelial growth within the substrate and/or on a surface of the substrate, placing the tool into a growth chamber, controlling the growth environment to induce growth of aerial mycelium from the fungus, and growing aerial mycelium from the fungus to form a preselected homogeneous aerial mycelium growth topology. In some aspects, the substrate and the tool can be configured to support aerial mycelial growth. In some aspects, the growth chamber can include a controlled growth environment conducive to aerial mycelial growth.

In some further aspects, placing can include selectively placing the at least one fungus in a preselected fungus pattern, wherein growing can include growing the aerial mycelium such that the preselected homogeneous aerial mycelium growth topology includes a preselected aerial mycelium growth pattern corresponding with the preselected fungus pattern.

In additional aspects, growing can include growing a plurality of intentionally positioned merged bulbous structures forming the preselected aerial mycelium growth pattern upon a growth surface of the aerial mycelium.

In some further aspects, the bulbous structures can be regularly-spaced with respect to each other.

In some aspects, the bulbous structures can form a mean native height on the growth surface relative to each other, and the coefficient of variation of the mean native height can be less than 2.

In some aspects, growing can include growing the aerial mycelium though a casing layer to form the preselected homogeneous aerial mycelium growth topology.

All of these embodiments are intended to be within the scope of the invention herein disclosed. These and other embodiments of the present invention will become readily apparent to those skilled in the art from the following detailed description of the preferred embodiments having reference to the attached figures, the invention not being limited to any particular preferred embodiment(s) disclosed.

### BRIEF DESCRIPTION OF THE DRAWINGS

The features and advantages of the invention described herein will become more fully apparent from the following description and appended claims, taken in conjunction with the accompanying drawings. These drawings depict only several embodiments in accordance with the disclosure and are not to be considered limiting of its scope. In the drawings, similar reference numbers or symbols typically identify similar components, unless context dictates otherwise. In some instances, the drawings may not be drawn to scale.

FIG. 1 illustrates positive gravitropism in extra-particle appressed mycelial growth.

FIG. 2 illustrates negative gravitropism in extra-particle aerial mycelial growth.

FIG. 3 illustrates a flow chart of substrate inoculation, substrate colonization, application, incubation, aerial mycelial growth, and aerial mycelium processing.

FIG. 4 illustrates a pattern of aerial mycelial growth resulting from a method to affect the topology thereof.

FIG. 5 illustrates horizontal airflow.

FIGS. 6A and 6B illustrate aerial mycelium morphologies from the control cultivation paradigm.

FIGS. 7A and 7B illustrate cross sectional schematic views of two examples of a growth matrix with extra-particle growth extending through one or more topology adjustment layers.

FIG. 8 shows a chart of treatments and their descriptions, which are various embodiments of the present disclosure, and which correspond to experimental conditions described in FIGS. 9-29.

FIGS. 9A-D illustrate an example of feed-grain application and positions.

FIGS. 10A-D illustrate a method for surface application of feed-grain spawn onto a substrate with a defined pattern.

FIGS. 11A-21D illustrate a visual timeline of growth morphology for each experimental condition.

FIGS. 22A-C illustrate an overhead view of three experimental material conditions at the conclusion of incubation.

FIGS. 23A-C illustrate intact sheets of aerial mycelium after extraction.

FIGS. 24A-D show graphs comparing physical characteristics of experimental conditions including total bone-dry mass yield (DM Yield), growth height (Z max), growth volume (Tissue Vol), and topological heterogeneity (RSE).

FIGS. 25A and 25B illustrate luminance height profiles as top-down views of 3D scans of each experimental condition, showing only tissue that grew above the surface of the substrate.

FIGS. 26A and 26B illustrate morphological segmentation of each of a plurality of point clouds based on their Z by XY luminance height profiles, where the panel is subdivided into the minimum number of discrete morphological features based on the eccentricity of the panel's topology.

FIGS. 27A and 27B illustrate mean segment area and number of discrete morphological segments based on image-based morphological analysis and segmentation.

FIGS. 28A-D illustrate relative differences between samples based on total morphological response (A: color coding by replicate ID; B: color coding by treatment; C: color coding by mean CO₂% and D: Euclidean distance between samples based on total morphological response).

FIG. 29 illustrates one experimental condition after completion of the growth cycle, physical removal (peeling) from the substrate, and drying for 24 hours at 110° F. in a commercial dehydrator to a final moisture content of <10%.

FIG. 30 illustrates a layout for cutting “dog bone” shaped specimens from an intact sheet of aerial mycelium to be used for evaluating tensile strength based on a standard testing protocol.

FIGS. 31A-D show the resulting mechanical data (tensile strength, modulus of elasticity, density, and displacement) for an experimental condition data set compared to a control condition data set.

FIGS. 32A-C show plots of tensile strength, modulus of elasticity, and displacement as a function of sample density.

FIG. 33 shows a summary table indicating increased density, increased displacement, decreased modulus of elasticity, and increased tensile strength for an experimental condition as compared to control conditions.

FIGS. 34A-E show morphological and mechanical responses based on application of a casing layer.

FIGS. 35A and 35B show observed morphology differences between samples with and without an uninoculated casing layer.

FIGS. 36A-C show measured physical differences between samples with and without an uninoculated casing layer.

FIGS. 37A and 37B show samples without a casing layer after 7 days of growth.

FIG. 38 shows a mid-section scan from a sample with grain application below a casing layer.

### DETAILED DESCRIPTION

The following discussion presents detailed descriptions of the several embodiments of growing aerial mycelium shown in the figures. These embodiments are not intended to be limiting, and modifications, variations, combinations, etc., are possible and within the scope of this disclosure.

The present disclosure provides for an aerial mycelium, methods of making an aerial mycelium, and uses thereof.

It is an object of the invention to provide an improved mycelium in the form of an aerial mycelium that is suitable for use as a textile, such as a leather-like material, or a petroleum-based material alternative, such as a foam.

It is another object of the invention to provide a method of making an aerial mycelium suitable for use as a textile, such as a leather-like material, or a petroleum-based material alternative, such as a foam.

It is yet another object of the invention to provide a product containing an aerial mycelium, and a method of making a product comprising an aerial mycelium, such as a textile, including a leather-like material, or a petroleum-based material alternative, including a foam.

It is another object of the invention to provide a mycelium-based product having a texture that is analogous to a textile or leather-like material.

Methods of making aerial mycelia of the present disclosure, methods of post-processing the aerial mycelia, and aerial mycelia and processed aerial mycelia obtained therefrom, can be adapted to prepare a wide variety of materials and products. Generally contiguous aerial mycelium panels can be implemented for applications such as, for example, materials and products that can be used to replace animal-derived or petroleum-based textiles, leather and leather-like materials and products, or to provide foam-like materials for use in upholstery, apparel, military gear, athletic gear, footwear, and the like.

In some aspects, the present disclosure provides for an aerial mycelium, and for methods of making an aerial mycelium, wherein the aerial mycelium is a growth product of a fungus. In some embodiments, the fungus is a species of the genus Agrocybe, Albatrellus, Armillaria, Agaricus, Bondarzewia, Cantharellus, Cerioporus, Climacodon, Cordyceps, Fistulina, Flammulina, Fomes, Fomitopsis, Fusarium, Grifola, Hericium, Hydnum, Hypomyces, Hypsizygus, Ischnoderma, Laetiporus, Laricifomes, Lentinula, Lentinus, Lepista, Meripilus, Morchella, Ophiocordyceps, Panellus, Piptoporus, Pleurotus, Polyporus, Pycnoporellus, Rhizopus, Schizophyllum, Stropharia, Tuber, Tyromyces, Wolfiporia, Ceriporiopsis, Chlorociboria, Daedalea, Daedaleopsis, Daldinia, Ganoderma, Hypoxylon, Inonotus, Lenzites, Omphalotus, Oxyporus, Phanerochaete, Phellinus, Polyporellus, Porodaedalea, Pycnoporus, Scytalidium, Stereum, Trametes or Xylaria. In some further embodiments, the fungus is a species of the genus Bondarzewia, Ceriporiopsis, Daedalea, Daedaleopsis, Fomitopsis, Ganoderma, Inonotus, Lenzites, Omphalotus, Oxyporus, Phellinus, Polyporellus, Polyporus, Porodaedalea, Pycnoporus, Stereum, Trametes or Xylaria. In some more particular embodiments, the fungus is selected from the group consisting of Bondarzewia berkeleyi, Daedalea quercina, Daedaleopsis spp., Daedaleopsis confragosa, Daedaleopsis septentrionalis, Fomitopsis spp., Fomitopsis cajanderi, Fomitopsis pinicola, Ganoderma spp., Ganoderma amboinense, Ganoderma applanatum, Ganoderma atrum, Ganoderma australe, Ganoderma brownii, Ganoderma capense, Ganoderma carnosum, Ganoderma cochlear, Ganoderma colossus, Ganoderma curtisii, Ganoderma donkii, Ganoderma formosanum, Ganoderma gibbosum, Ganoderma hainanense, Ganoderma hoehnelianum, Ganoderma japonicum, Ganoderma lingzhi, Ganoderma lobatum, Ganoderma lucidum, Ganoderma multipileum, Ganoderma oregonense, Ganoderma pfeifferi, Ganoderma resinaceum, Ganoderma sessile, Ganoderma sichuanense, Ganoderma sinense, Ganoderma tropicum, Ganoderma tsugae, Ganoderma tuberculosum, Ganoderma weberianum, Inonotus spp., Inonotus obliquus, Inonotus hispidus, Inonotus dryadeus, Inonotus tomentosus, Lenzites betulina, Phellinus spp., Phellinus igniarius, Phellinus gilvus, Polyporus spp., Polyporus squamosus, Polyporus badius, Polyporus umbellatus, Polyporus squamosus, Polyporus tuberaster, Polyporus arcularius, Polyporus alveolaris, Polyporus radicatus, Porodaedalea pini, Pycnoporus spp., Pycnoporus spp., Pycnoporus sanguineus, Pycnoporus cinnabarinus, Stereum spp., Stereum ostrea, Stereum hirsutum, Trametes spp., Trametes versicolor, Trametes elegans, Trametes suaveolens, Trametes hirsuta, Trametes gibbosa, Trametes ochracea, Trametes villosa, Trametes cubensis and Trametes pubescens. In some other embodiments, the fungus is a pigment-producing fungus of a genus selected from the group consisting of Chlorociboria, Daldinia, Hypoxylon, Phanerochaete and Scytalidium. In yet some other embodiments, the fungus is a species of the genus Ganoderma. In some further embodiments, the fungus is Ganoderma spp., Ganoderma amboinense, Ganoderma applanatum, Ganoderma atrum, Ganoderma australe, Ganoderma brownii, Ganoderma capense, Ganoderma carnosum, Ganoderma cochlear, Ganoderma colossus, Ganoderma curtisii, Ganoderma donkii, Ganoderma formosanum, Ganoderma gibbosum, Ganoderma hainanense, Ganoderma hoehnelianum Ganoderma japonicum, Ganoderma lingzhi, Ganoderma lobatum, Ganoderma lucidum, Ganoderma multipileum, Ganoderma oregonense, Ganoderma pfeifferi, Ganoderma resinaceum, Ganoderma sessile, Ganoderma sichuanense, Ganoderma sinense, Ganoderma tropicum, Ganoderma tsugae, Ganoderma tuberculosum or Ganoderma weberianum.

Aerial mycelium is morphologically composed of variably expressed structures (e.g., bulbous structures) with varying degrees of diffusion within and between one another, and in height, with respect to each other. This may be referred to more generally as the topology of growth. The variable and eccentric expression of bulbous features and variable tissue density within and between bulbous features represents a challenge for textile applications. For example, tensile failure can selectively occur when morphological “bulb” forms become too discrete, due to a lack of cross-linking at the intersections between these forms. This can lead to variable failure modes and reduced tensile strengths.

However, a perfect homogeneity may not be desired. Too much homogeneity can look artificial, for example, in a textile product. Thus, some heterogeneity may be desired. Additionally, too high of density in the raw material can lead to difficulty with re-wetting and chemistry penetration. In this case it may be more desirable to have a lower density raw material at a defined mass per unit area, upon which chemical penetration can be performed, and further densification can be achieved through compression. A more acute problem than general heterogeneity (assuming adequate crosslinking between discrete “bulb” forms) is geometrically irregular expression of the given “bulb” forms.

Given the above, a desirable process would be to control topology in order to provide a more homogeneous (but still heterogeneous to a degree) lower density mat with geometrically normalized morphology and diffusion and crosslinking between “bulb” forms.

Methods for affecting the growth topology of aerial mycelium, including, for example, growing aerial mycelium with a desired level of homogeneity, are described herein. The increased homogeneity can include a reduced number of discrete bulbous features, or a lack of discrete bulbous features, to improve quality and reduce tensile failure.

Methods for growing aerial mycelium with increased geometric regularity is also described herein. The geometric regularity can include expression of bulbous features in a defined geometry such that textural, aesthetic, and mechanical qualities are reproducible.

### Definitions Related to Mycelium and its Characterization

“Mycelium” as used herein refers to a connective network of fungal hyphae.

“Hyphae” as used herein refers to branched filament vegetative cellular structures that are interwoven to form mycelium.

“Fruiting body” as used herein refers to a stipe, pileus, gill, pore structure, or a combination thereof.

“Extra-particle mycelial growth” (EPM) as used herein refers to mycelial growth, which can be either appressed or aerial.

“Extra-particle appressed mycelial growth” as used herein refers to a distinct mycelial growth that is surface-tracking (thigmotropic), is determinate in growth substantially orthogonal to the surface of a growth matrix, is indeterminate in growth substantially parallel to the surface of the growth matrix, and which can exhibit positive gravitropism.

“Appressed mycelium” as used herein refers to a continuous mycelium obtained from extra-particle appressed mycelial growth, and which is substantially free of growth matrix.

“Determinate growth” as used herein refers to growth that occurs until a maximum final dimension is achieved while growth continues to occur in other dimensions. Either determinate or indeterminate mycelial growth above the surface of a growth matrix defines a mycelium's native thickness.

“Indeterminate growth” as used herein refers to growth that expands indefinitely in a given direction as long as mycelial growth is occurring.

“Positive gravitropism” as used herein refers to growth that preferentially occurs in the direction of gravity.

“Extra-particle aerial mycelial growth” as used herein refers to a distinct mycelial growth that occurs away from and outward from the surface of a growth matrix, and which can exhibit negative gravitropism. In a geometrically unrestricted scenario, extra-particle aerial mycelial growth could be described as being positively gravitropic, or neutrally gravitropic, aerial, and radial in which growth will expand in all directions from its point source. In some embodiments, external forces, such as airflow, can be applied towards (e.g., approximately perpendicular to) the growth substrate, and in some embodiments, through the growth substrate, for example, to create downward aerial mycelium growth in the direction of gravity.

“Aerial mycelium” as used herein refers to mycelium obtained from extra-particle aerial mycelial growth, and which is substantially free of growth matrix.

“Growth surface” as used herein refers to an outer-facing surface of the mycelium, extra-particle aerial mycelial growth or aerial mycelium.

“Negative gravitropism” as used herein refers to mycelial growth that preferentially occurs in the direction away from gravity. As disclosed herein, extra-particle aerial mycelial growth exhibits negative gravitropism. Without being bound by any particular theory, this may be attributable at least in part to the geometric restriction of the growth format, wherein an uncovered tool having a bottom and side walls contains a growth matrix. With such geometric restriction, growth will primarily occur along the unrestricted dimension(s), which in the scenario is primarily vertically (negatively gravitropic).

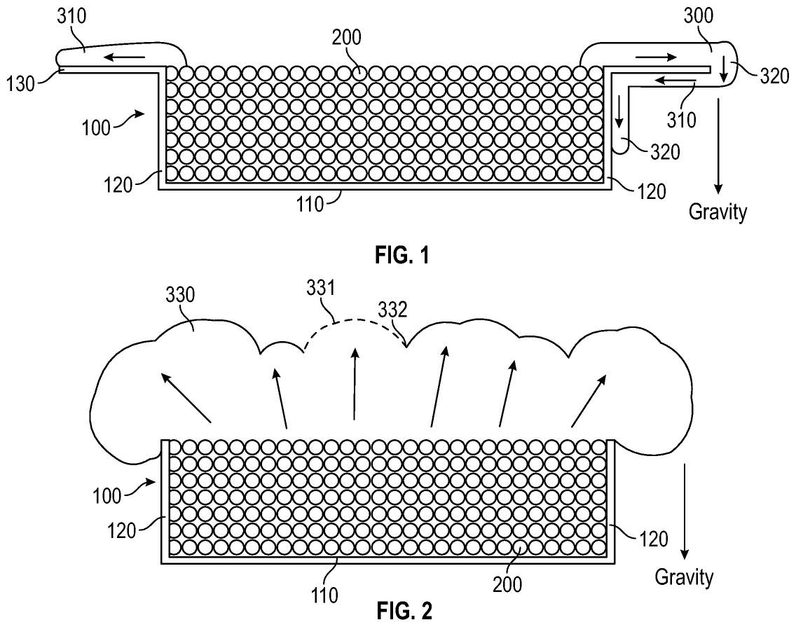

To better understand these terms, an embodiment of positive gravitropism in extra-particle appressed mycelial growth of the present disclosure is illustrated in FIG. 1. Referring to FIG. 1, a growth unit consists of a single tray container 100 with a bottom 110 and side walls 120, with horizontally oriented rigid surfaces 130 placed as a skirt oriented at the lip of the tray container 100. The tray container 100 contains growth matrix 200. In the absence of physical water mist deposition, extra-particle mycelial growth (EPM) 300 expands along the horizontally oriented rigid surfaces 130 as a function of a preference for surface-tracking growth 310. In this case, if and when the expanding EPM 300 reaches the boundary of the horizontally oriented skirt, EPM will default to a combination of surface-tracking 310 and positive gravitropism 320, continuing to expand along the underside of the skirt 130 or the exterior surfaces of the sidewalls 120 of the tray container 100.

To better understand these terms, an embodiment of negative gravitropism in extra-particle aerial mycelial growth of the present disclosure is illustrated in FIG. 2. Referring to FIG. 2, the growth unit consists of a single tray container 100 with a bottom 110 and side walls 120. The tray container 100 contains growth matrix 200. Aqueous mist (not shown) is deposited directly onto the exposed growth matrix surface 200, resulting in EPM initiating across the exposed surface. With continued physical aqueous mist deposition, EPM continues to expand forming a contiguous, semi-contiguous, or dis-contiguous volume of extra-particle aerial mycelial growth 330 with discrete bulbous features 331, and corresponding depressions 332 therebetween, as a combined function of mist deposition rates and mean mist deposition rates. It will be understood that embodiments herein that reference a “tray” or “tray container” can be implemented with another type of tool (e.g., containers, carrier sheets, conveyer belts, shelves (such as open shelving), beds or other surfaces or containers, etc.).

“Mycelium-based” as used herein refers to a composition substantially comprising mycelium.

As used herein, “bulbous” refers to a morphological feature having a bulb-like morphology that may be spherical or eccentric.

As used herein, “growth topology” refers to the overall topological character and morphology of an aerial mycelium panel as a function of the pattern of expression and level of fusion between bulbous features.

In a further aspect, the present disclosure provides for an aerial mycelium characterized as having particular physicochemical properties.

“Homogeneous” as used herein refers to the topology of growth of the aerial mycelium. In some embodiments, aerial mycelium is morphologically composed of variably expressed structures (e.g., bulbous structures) with varying degrees of diffusion within and between one another, and in height, with respect to each other. This may be referred to more generally as the topology of growth. The variable and eccentric expression of bulbous features and variable tissue density within and between bulbous features represents a challenge for example, in textiles applications. For example, tensile failure can selectively occur when morphological “bulb” forms become too discrete, due to a lack of cross-linking at the intersections between these forms, which can lead to variable failure modes and reduced physical strength. Conversely, increased homogeneity can increase tensile strength, for example, by increasing cross-linking. Compressing an aerial mycelium that has increased homogeneity can further increase tensile strength. Given the above, a desirable process would be to control topology to provide a more normalized aerial mycelium morphology and crosslinking between “bulb” forms.

In some further embodiments, increased homogeneity can include a reduced number of discrete bulbous features, a lack of discrete bulbous features, or a plurality of discrete bulbous features wherein such bulbous features have so merged that the overall topography of the overall material includes few if any visually discernible low or high points in the vertical (Z) direction as observed by an observer without visual impairment from a distance of about 12 inches.

In some embodiments, discrete bulbous features are quantified per morphological segmented area (mm²) of the growth surface. In some further embodiments, the morphological segmented area is between about 1000 mm² and 3000 mm², or in some embodiments, between about 1500 mm² and 2900 mm². In some further embodiments, the sizes of discrete bulbous features are measured by one of morphological segment perimeter, morphological segment maximum Feret diameter, or morphological segment inscribed disc radius. In some embodiments, the morphological segment perimeter of discrete bulbous features is between 90 mm and 230 mm, or in some embodiments, between about 100 mm and 200 mm. In some embodiments, the morphological segment maximum Feret diameter is between 30 mm and 80 mm, or in some embodiments, between about 50 mm and 80 mm. In some embodiments, the morphological segment inscribed disc radius is between 6 mm and 20 mm, or in some embodiments, between about 9 mm and 19 mm.

In some embodiments, the mean of the “native height”, i.e., the mean of the difference in distance between the discernable low points (e.g., depressions 332 between bulbs 331 as illustrated in FIG. 2) and high points (e.g., the highest point on the bulbs) in the vertical (Z) direction of the growth surface, is approaching 0 mm, is between about 0 mm and about 65 mm, is between about 1 mm and about 6 mm, is between about 6 mm and about 30 mm, or is between about 13 mm and about 30 mm. In some further embodiments, the maximum native height is between about 20 mm and about 63 mm, and in some embodiments, between about 37 mm and about 63 mm. In some further embodiments, the coefficient of variation of the difference between the discernable low or high points in the vertical (Z) direction (e.g., the mean native height) is less than 2. In some embodiments, the coefficient of variation of the difference between the discernable low or high points in the vertical (Z) direction (e.g., the mean native height) is less than 0.75. In some embodiments, the coefficient of variation of the difference between the discernable low or high points in the vertical (Z) direction (e.g., the mean native height) is less than 0.5. In some embodiments, the coefficient of variation of the difference between the discernable low or high points in the vertical (Z) direction (e.g., the mean native height) is less than 0.25. In some embodiments, the coefficient of variation the difference between the discernable low or high points in the vertical (Z) direction (e.g., the mean native height) is less than 1.

In some embodiments, a minimum number of discrete morphological features is preferred to reduce the coefficient of variation of height. In some further embodiments, perfect homogeneity can result in a product with undesirable aesthetic and haptic qualities, for example, in a textile product. Thus, in some embodiments, varying degrees of inhomogeneity may be desired. In some embodiments, methods for affecting the growth topology of aerial mycelium, including, for example, growing aerial mycelium with a desired level of homogeneity, are described herein. In some embodiments, methods for growing aerial mycelium with increased geometric regularity is also described herein. The geometric regularity can include expression of bulbous features in a defined geometry such that textural, aesthetic, and mechanical qualities are reproducible. An aerial mycelium with increased homogeneity, upon being compressed, can result in a compressed aerial mycelium with greater tensile strength than a corresponding compressed aerial mycelium with decreased homogeneity.

A “native” property as used herein refers to a property associated with a mycelium obtained after an incubation time period has elapsed and upon subsequent removal of the mycelial growth from a growth matrix, and prior to any optional environmental, physical, or other post-processing step(s) or excursion(s), whether intentional or unintentional, that substantially alters the property. In some aspects, the present disclosure provides for a mycelium characterized as having one or more “native” properties. In some further aspects, the native property can be a native density, a native thickness, a native nutritional content, a native moisture content, a native compressive modulus, a native height on a growth surface of the mycelium, or another native property as described herein, which is not intended to be limiting. In a nonlimiting example, an environmental step can be a drying step, such as one that reduces the aerial mycelial native moisture content to less than about 80% (w/w). In another nonlimiting example, a physical step can be a compression step that substantially reduces the thickness of an aerial mycelium. Unless stated otherwise, any reference to “height” with respect to the embodiments herein are used interchangeably with “native height.”

“Native moisture content” as used herein refers to the moisture content of a mycelium obtained after an incubation time period has elapsed and the resulting mycelial growth has been removed from a growth matrix, and prior to performing any optional environmental, physical, or other post-processing step(s) that may increase or decrease the moisture content of the mycelium so obtained. In some embodiments, a mycelium of the present disclosure is characterized as having a native moisture content. In some embodiments, the native moisture content is expressed as a mean native moisture content.

In some embodiments, an aerial mycelium of the present disclosure can have a native moisture content of at most about 75% (w/w). In some embodiments, an aerial mycelium can have a native moisture content of greater than about 80% (w/w). In some further embodiments, an aerial mycelium of the present disclosure can have a native moisture content of at least about 85% (w/w), or at least about 90% (w/w). In some embodiments, an aerial mycelium of the present disclosure can have a native moisture content of at most about 95% (w/w), at most about 94% (w/w) or at most about 93% (w/w). In some more particular embodiments, an aerial mycelium can have a native moisture content of about 81% (w/w), about 82% (w/w), about 83% (w/w), about 84% (w/w), about 85% (w/w), about 86% (w/w), about 87% (w/w), about 88% (w/w), about 89% (w/w), about 90% (w/w), about 91% (w/w), about 92% (w/w), about 93% (w/w), about 94% (w/w) or about 95% (w/w), or any range therebetween. In some embodiments, an aerial mycelium can have a native moisture content within a range of about 75% (w/w) to about 95% (w/w), about 80% (w/w) to about 95% (w/w), about 75% (w/w) to about 93% (w/w) or about 80% (w/w) to about 93% (w/w). Typically, an aerial mycelium of the present disclosure has a native moisture content of about 90% (w/w). In some embodiments, an appressed mycelium of the present disclosure can have a native moisture content of not more than about 80% (w/w), for example, within a range of about 70% (w/w) to about 80% (w/w).

In some embodiments, a mycelium of the present disclosure is characterized as having a native thickness. In some embodiments, the native thickness is expressed as a mean native thickness as determined from sampling over the volume of the mycelium. Typically, the native mycelial thickness is determined from a mycelium obtained after an incubation time period has elapsed and the resulting extra-particle mycelial growth has been removed from a growth matrix, and prior to performing any optional environmental, physical, or other post-processing step(s) that may compress or expand the thickness of the mycelium so obtained.

In some aspects, an aerial mycelium of the present disclosure has a native thickness of greater than about 10 mm. In some embodiments, an aerial mycelium of the present disclosure can have a native thickness of at least about 15 mm, at least about 20 mm, at least about 25 mm, at least about 30 mm, at least about 35 mm, at least about 40 mm, at least about 45 mm, at least about 50 mm, at least about 55 mm, at least about 60 mm, at least about 65 mm or at least about 70 mm. In some embodiments, the native thickness is a mean native thickness. Thus, in some further embodiments, an aerial mycelium of the present disclosure can have a mean native thickness of at least about 15 mm, at least about 20 mm, at least about 25 mm, at least about 30 mm, at least about 35 mm, at least about 40 mm, at least about 45 mm, at least about 50 mm, at least about 55 mm or at least about 60 mm. In some embodiments, the native thickness is a median native thickness. Thus, in some further embodiments, an aerial mycelium of the present disclosure can have a median native thickness of at least about 15 mm, at least about 20 mm, at least about 25 mm, at least about 30 mm, at least about 35 mm, at least about 40 mm, at least about 45 mm, at least about 50 mm, at least about 55 mm, at least about 60 mm, or at least about 65 mm. In some embodiments, the native thickness is a maximum native thickness. Thus, in some further embodiments, an aerial mycelium of the present disclosure can have a maximum native thickness of at most about 150 mm, at most about 125 mm, at most about 100 mm, at most about 95 mm, at most about 90 mm, or at most about 85 mm.

In some other aspects, at least a portion of an aerial mycelium (or an aerial mycelial panel) of the present disclosure can have a native thickness of greater than about 10 mm. In some embodiments, at least a portion of an aerial mycelium of the present disclosure can have a native thickness of at least about 15 mm, at least about 20 mm, at least about 25 mm, at least about 30 mm, at least about 35 mm, at least about 40 mm, at least about 45 mm, at least about 50 mm, at least about 55 mm, at least about 60 mm, at least about 65 mm, at least about 70 mm, at least about 75 mm or at least about 80 mm. In some more particular embodiments, the portion can be at least about 10%, at least about 20%, at least about 30%, at least about 40%, at least about 50%, at least about 60% at least about 70%, at least about 80% or at least about 90% of the aerial mycelium.

Thus, in some embodiments, the present disclosure provides for an aerial mycelium, wherein at least 25% of the aerial mycelium (i.e., at least 25% of a single aerial mycelial panel) can have a native thickness of at least about 20 mm, at least about 25 mm, at least about 30 mm, at least about 35 mm, at least about 40 mm, at least about 45 mm, at least about 50 mm, at least about 55 mm, at least about 60 mm, at least about 65 mm or at least about 70 mm. In some embodiments, the present disclosure provides for an aerial mycelium, wherein at least 50% of the aerial mycelium can have a native thickness of at least about 20 mm, at least about 25 mm, at least about 30 mm, at least about 35 mm, at least about 40 mm, at least about 45 mm, at least about 50 mm, at least about 55 mm, at least about 60 mm, at least about 65 mm or at least about 70 mm. In some embodiments, the present disclosure provides for an aerial mycelium, wherein at least 75% of the aerial mycelium can have a native thickness of at least about 20 mm, at least about 25 mm, at least about 30 mm, at least about 35 mm, at least about 40 mm, at least about 45 mm, at least about 50 mm, at least about 55 mm, or at least about 60 mm. In a nonlimiting example, the present disclosure provides for an aerial mycelium, wherein 75% of the aerial mycelium has a native thickness of about 54 mm, 50% of the aerial mycelium has a native thickness of about 66 mm, and 25% of the aerial mycelium has a native thickness of about 70 mm.

In some further embodiments, an aerial mycelium of the present disclosure can have a native thickness of at least about 20 mm, at least about 30 mm or at least about 40 mm over at least 60% of the aerial mycelium. In yet further embodiments, an aerial mycelium of the present disclosure can have a native thickness of at least about 20 mm, at least about 30 mm or at least about 40 mm over at least 70% of the aerial mycelium. In even more particular embodiments, an aerial mycelium of the present disclosure can have a native thickness of at least about 20 mm or at least about 30 mm over at least 70% of the aerial mycelium. In some more particular embodiments still, an aerial mycelium of the present disclosure can have a native thickness of at least about 20 mm over at least 80% of the aerial mycelium. In some preferred embodiments, an aerial mycelium of the present disclosure can have a native thickness of at least about 20 mm over at least 90% of the aerial mycelium.

In some aspects, a mycelium of the present disclosure is characterized as having a surface area. The surface area of an aerial mycelium of the present disclosure can be characterized as the area of the aerial mycelium that occupies the plane that is substantially orthogonal to the direction of mycelial growth.

In some aspects, an aerial mycelium of the present disclosure can have a surface area that is at least about 80% of the surface area of the growth matrix or is at least about 90% of the surface area of the growth matrix. In some further aspects, an aerial mycelium of the present disclosure can have a surface area that is at most about 125% of the surface area of the growth matrix. In some further aspects, an aerial mycelium of the present disclosure can have a surface area of at least about 1 square inch. In some yet further aspects, an aerial mycelium of the present disclosure can have a surface area of at most about 2,000 square feet.

In some aspects, a mycelium of the present disclosure is characterized as a contiguous mycelium. A contiguous mycelium of the present disclosure can be obtained by removing a contiguous extra-particle mycelial growth from a growth matrix as a contiguous object. “Contiguous” as used herein in connection with an extra-particle aerial mycelial growth or an aerial mycelium refers to an extra-particle aerial mycelial growth or an aerial mycelium having a contiguous volume, wherein the contiguous volume is at least about 15 cubic inches, has a series of linked hyphae over the contiguous volume, or both. In some embodiments, an aerial mycelium of the present disclosure can have a contiguous volume of at least about 150 cubic inches, at least about 300 cubic inches or more. In some embodiments, a contiguous aerial mycelium of the present disclosure can be obtained by removing a contiguous extra-particle aerial mycelial growth from a growth matrix as a contiguous 3-dimensional object, which may be referred to herein as a panel.

In some embodiments, a mycelium of the present disclosure is characterized as having a native density. In some embodiments, the native density is expressed as a mean native density as determined from sampling over the volume of the mycelium. “Native density” as used herein in connection with an aerial mycelium refers to the density of an aerial mycelium having a native moisture content of at least about 80% (w/w), or at least about 90% (w/w), and at most about 100% (w/w). Typically, the native density is determined from a mycelium obtained after an incubation time period has elapsed and the resulting mycelial growth has been removed from a growth matrix, and prior to performing any optional environmental, physical, or other post-processing step(s) that may compress or expand the aerial mycelium so obtained. An environmental step can be a drying step that reduces the aerial mycelial native moisture content to less than about 80% (w/w).

Thus, in some embodiments, an aerial mycelium of the present disclosure can have a mean native density of no greater than about 70 pounds per cubic foot (pcf). In some embodiments, an aerial mycelium of the present disclosure can have a mean native density within a range of about 0.05 to about 70 pcf. In a further embodiment, an aerial mycelium of the present disclosure can have a mean native density within a range of about 0.05 to about 15 pcf. As a non-limiting example, an aerial mycelium of the present disclosure can have a low native density of about 0.06 pcf.

In some other embodiments, an aerial mycelium of the present disclosure can have a mean native density within a range of about 1 pcf to about 70 pcf. In some further embodiments, the aerial mycelium can have a mean native density of at least about 1 pcf, at least about 2 pcf, at least about 3 pcf, at least about 4 pcf, at least about 5 pcf, at least about 6 pcf, at least about 7 pcf, at least about 8 pcf, at least about 9 pcf or at least about 10 pcf. In yet some further embodiments, the aerial mycelium can have a mean native density of at most about 60 pcf, at most about 55 pcf, at most about 50 pcf, at most about 45 pcf, at most about 40 pcf, at most about 35 pcf, at most about 30 pcf, at most about 25 pcf, at most about 20 pcf or at most about 15 pcf. In some embodiments, an aerial mycelium of the present disclosure has a mean native density within a range of about 0.1 pcf to about 50 pcf, about 0.1 pcf to about 45 pcf, about 0.1 pcf to about 40 pcf, about 0.1 pcf to about 35 pcf, about 0.1 pcf to about 30 pcf, about 0.1 pcf to about 25 pcf, about 0.1 pcf to about 20 pcf, about 0.1 pcf to about 15 pcf, about 0.1 pcf to about 10 pcf, about 0.1 pcf to about 8 pcf, about 0.1 pcf to about 7 pcf, about 0.1 pcf to about 6 pcf, or about 0.1 pcf to about 5 pcf. In some embodiments, an aerial mycelium of the present disclosure has a mean native density within a range of about 1 pcf to about 50 pcf, about 1 pcf to about 45 pcf, about 1 pcf to about 40 pcf, about 1 pcf to about 35 pcf, about 1 pcf to about 30 pcf, about 1 pcf to about 25 pcf, about 1 pcf to about 20 pcf, about 1 pcf to about 15 pcf, about 1 pcf to about 10 pcf, about 1 pcf to about 8 pcf, about 1 pcf to about 7 pcf, about 1 pcf to about 6 pcf, or about 1 pcf to about 5 pcf. In some further embodiments, an aerial mycelium of the present disclosure has a mean native density within a range of about 2 pcf to about 50 pcf, about 2 pcf to about 45 pcf, about 2 pcf to about 40 pcf, about 2 pcf to about 35 pcf, about 2 pcf to about 30 pcf, about 2 pcf to about 25 pcf, about 2 pcf to about 20 pcf, about 2 pcf to about 15 pcf, about 2 pcf to about 10 pcf, about 2 pcf to about 8 pcf, about 2 pcf to about 7 pcf, about 2 pcf to about 6 pcf, or about 2 pcf to about 5 pcf. In some yet further embodiments, an aerial mycelium of the present disclosure has a mean native density within a range of about 3 pcf to about 50 pcf, about 3 pcf to about 45 pcf, about 3 pcf to about 40 pcf, about 3 pcf to about 35 pcf, about 3 pcf to about 30 pcf, about 3 pcf to about 25 pcf, about 3 pcf to about 20 pcf, about 3 pcf to about 15 pcf, about 3 pcf to about 10 pcf, about 3 pcf to about 8 pcf, about 3 pcf to about 7 pcf, about 3 pcf to about 6 pcf, or about 3 pcf to about 5 pcf. In some more particular embodiments, an aerial mycelium of the present disclosure has a mean native density of about 0.05 pcf, about 1 pcf, about 2 pcf, about 3 pcf, about 4 pcf, about 5 pcf, about 6 pcf, about 7 pcf, about 8 pcf, about 9 pcf, about 10 pcf, about 11 pcf, about 12 pcf, about 13 pcf, about 14 pcf or about 15 pcf, or any range therebetween.

In yet other embodiments, an aerial mycelium of the present disclosure can have a mean native density of at most about 15 pcf. In some embodiments, an aerial mycelium can have a mean native density within a range of about 0.1 pcf to about 15 pcf. In some embodiments, an aerial mycelium can have a mean native density of at most about 10 pcf, or at most about 5 pcf.

In some embodiments, a mycelium of the present disclosure is characterized as having a dry density. In some embodiments, the dry density is expressed as a mean dry density as determined from sampling over the volume of the mycelium. “Dry density” (or bone-dry density) as used herein refers to the density of a mycelium having a moisture content of no greater than about 10% (w/w). Typically, the dry density of a mycelium is determined after removing mycelial growth from a growth matrix to obtain a mycelium, and subsequently drying the mycelium to a moisture content of no greater than about 10% (w/w).

Thus, in some embodiments, an aerial mycelium of the present disclosure can have a mean dry density of at most about 7 pcf, at most about 6 pcf or at most about 5 pcf. In some embodiments, an aerial mycelium of the present disclosure can have a mean dry density within a range of about 0.05 pcf to about 7 pcf, about 0.05 pcf to about 6 pcf, about 0.05 to about 5 pcf, about 0.05 to about 4 pcf, about 0.05 to about 3 pcf, about 0.1 pcf to about 7 pcf, about 0.1 to about 6 pcf, about 0.1 to about 5 pcf, about 0.1 to about 4 pcf or about 0.1 to about 3 pcf. In some further embodiments, an aerial mycelium of the present disclosure has a mean dry density within a range of about 0.1 pcf to about 2 pcf. In some more particular embodiments, an aerial mycelium of the present disclosure has a mean dry density of about 0.1 pcf, about 0.2 pcf, about 0.3 pcf, about 0.4 pcf, about 0.5 pcf, about 0.6 pcf, about 0.7 pcf, about 0.8 pcf, about 0.9 pcf, about 1.0 pcf, about 1.1 pcf, about 1.2 pcf, about 1.3 pcf, about 1.4 pcf, about 1.5 pcf, about 1.6 pcf, about 1.7 pcf, about 1.8 pcf, about 1.9 pcf or about 2 pcf, or any range therebetween.

In some aspects, an aerial mycelium of the present disclosure can be further characterized by its hyphal width. In some embodiments, an aerial mycelium of the present disclosure has a mean hyphal width of no greater than about 20 microns (um), or no greater than about 15 microns. In some embodiments, an aerial mycelium of the present disclosure has a mean hyphal width within a range of about 0.1 micron to about 20 microns, about 0.1 micron to about 15 microns, or about 0.2 microns to about 15 microns, or any range between each of these values.

“Open volume” as used herein refers to the ratio of the volume of interstices of a mycelium to the volume of its mass. In some aspects, an aerial mycelium of the present disclosure can be characterized as having a “volume fraction,” which corresponds to the open volume expressed as a percentage. In some embodiments, an aerial mycelium of the present disclosure can have a volume fraction of at least about 50% (v/v), at least about 60%, or at least about 70% (v/v). In some embodiments, an aerial mycelium of the present disclosure can have a volume fraction within a range of about 50% to about 90%, or about 60% to about 80%. In some embodiments, the aerial mycelium having a volume fraction is a dried aerial mycelium. In some further embodiments, the dried aerial mycelium has a moisture content of less than about 10% (w/w).

As disclosed herein, an aerial mycelium of the present disclosure comprises a growth-grain. As further disclosed herein, an aerial mycelium can be characterized by its direction of mycelial growth. The growth-grain is generally aligned along a first axis, which may be referred to herein as an “aerial mycelial growth axis.” The orientation of the growth-grain may be evident at a macroscopic scale. The orientation of the growth-grain may be made more evident by the ease with which the aerial mycelium panel tears along this growth-grain, in analogy to the grain of a cut of meat. When looking microscopically, the growth-grain may be visible as a function of aggregations of hyphae that are oriented into larger aligned structures. Accordingly, physical properties of an aerial mycelium of the present disclosure can vary depending on how a physical (e.g., a mechanical) test or step is performed relative to the growth-grain or to the first axis. In some non-limiting embodiments, a physical property of an aerial mycelium can be assessed in a direction parallel to the first axis, in a direction perpendicular to the first axis, or both. In other non-limiting examples, a physical property of an aerial mycelium can be assessed with the growth-grain, against the growth-grain, or both. Such physical properties can include Kramer shear force, ultimate tensile strength and compressive modulus, compressive stress, and the like. In some embodiments, an aerial mycelium of the present disclosure can have a native Kramer shear force of greater than about 100 kg/g of aerial mycelium.

Hyphal alignments can be measured by methods known in the art (e.g., Boudaoud A. et al., FibrilTool, an ImageJ plug-in to quantify fibrillar structures in raw microscopy images, Nature Protocols, 9, 457-463, 2014, the entire contents of which are hereby incorporated by reference in their entirety to the extent not inconsistent with the content of this disclosure), which quantify the amount (i.e., outputs strengths of alignment along a single axis) of hyphal alignment as fractional anisotropy. An aerial mycelium of the present disclosure can have a fractional anisotropy of at least about 5%, or at least about 10%, and can have a fractional anisotropy of at most about 40%.

In some embodiments, an aerial mycelium of the present disclosure is characterized as having an ultimate tensile strength. In some embodiments, an aerial mycelium can have an ultimate tensile strength in a dimension substantially parallel to the growth-grain after drying the aerial mycelium to a final moisture content of less than about 10% (w/w), wherein the ultimate tensile strength in the dimension substantially parallel to the growth-grain after the drying of the aerial mycelium to a final moisture content of less than about 10% (w/w) is no greater than about 50 pounds per square inch (psi). In some embodiments, the ultimate tensile strength in the dimension substantially parallel to the growth-grain after the drying of the aerial mycelium to a final moisture content of less than about 10% (w/w) is no greater than about 40 psi.

In some embodiments, an aerial mycelium of the present disclosure is characterized as having a modulus of elasticity. In some embodiments, an aerial mycelium can have a modulus of elasticity of no greater than about 150 psi, of no greater than about 125 psi, or of no greater than about 100 psi.

In some embodiments, there is provided a batch of aerial mycelia. “Batch” as used herein refers to a quantity of goods produced at one time, wherein the quantity is at least two (2). In some embodiments, the quantity is at most about 10,000, at most about 5,000, at most about 1000, at most about 500, at most about 100, at most about 50, at most about two dozen or at most about a dozen. A batch of aerial mycelia of the present disclosure can be produced in a growth chamber or other system configured for growing aerial mycelia, or another controlled growth environment. In some embodiments, a batch of aerial mycelia of the present disclosure is produced under a predetermined set of growth conditions.

Thus, in some embodiments, there is provided a batch of aerial mycelia (or aerial mycelial panels). In some embodiments, greater than 50% of the aerial mycelia (or aerial mycelial panels) in said batch conform to having one or more properties. Non-limiting examples of said properties include a native density, a native moisture content, a native thickness, a native volume, absence of a fruiting body, a native compressive modulus, a native compressive stress, a native ultimate tensile strength and/or a native Kramer shear force, wherein each said property can have a preestablished value or range of values. In some further embodiments, greater than 50% of the aerial mycelia (or aerial mycelial panels) in the batch conform to at least two, at least three, at least four, at least five, at least six or more of said properties. In some embodiments, at least about 75% or more of the aerial mycelia (or aerial mycelial panels) in a batch conforms to having at least one, two, three, four, five, six or more of said properties. In some embodiments, an aerial mycelium of a batch of aerial mycelia (or aerial mycelial panels) can have one or more of said properties that are predetermined, e.g., by establishing a set of growth conditions and target values or ranges of values prior to making the aerial mycelium or batch of aerial mycelia.

Any of the methods herein can be implemented to make a batch of aerial mycelium, wherein greater than 50% of the panels in the batch comprise an aerial mycelium according to the method being implemented. In some implementations, greater than 50% of the panels in the batch are suitable for use (e.g., are for use) in the manufacture of a textile product, including a leather-like product. In some implementations, the textile product, including the leather-like product, is a mycelium-based textile product.

### Definitions and Methods Related to Materials for Growing Mycelium

“Substrate” as used herein refers to a material or surface thereof, from or on which an organism lives, grows, and/or obtains its nourishment. In some embodiments, a substrate provides sufficient nutrition to the organism under target growth conditions such that the organism can live and grow without providing the organism a further source of nutrients. A variety of substrates are suitable to support the growth of an aerial mycelium of the present disclosure. Suitable substrates are disclosed, for example, in US20200239830A1, the entire contents of which are hereby incorporated by reference in their entirety to the extent not inconsistent with the content of this disclosure. In some embodiments, the substrate is a natural substrate. Non-limiting examples of a natural substrate include a lignocellulosic substrate, a cellulosic substrate, or a lignin-free substrate. A natural substrate can be an agricultural waste product or one that is purposefully harvested for the intended purpose of food production, including mycelial-based food production. Further non-limiting examples of substrates suitable for supporting the growth of mycelia of the present disclosure include soy-based materials, oak-based materials, maple-based materials, corn-based materials, seed-based materials and the like, or combinations thereof. The materials can have a variety of particle sizes, as disclosed in US20200239830A1, and occur in a variety of forms, including shavings, pellets, chips, flakes, or flour, or can be in monolithic form. Non-limiting examples of suitable substrates for the production of mycelia of the present disclosure include corn stover, maple flour, maple flake, maple chips, soy flour, chickpea flour, millet seed flour, oak pellets, soybean hull pellets and combinations thereof. Additional useful substrates for the growth of mycelia are disclosed herein. A substrate can comprise a solid (e.g., discrete solid particles) and/or a liquid.

“Growth media” or “growth medium” as used herein refers to a substrate and an optional further source of nutrition that is the same as or different from the substrate, wherein the substrate, the nutrition source, or both are intended for fungal consumption to support mycelial growth.

“Growth matrix” as used herein refers to a matrix containing a growth medium and a fungus. In some embodiments, the fungus is provided as a fungal inoculum; thus, in such embodiments, the growth matrix comprises an inoculated substrate. In other embodiments, the growth matrix comprises a colonized substrate, as defined below. Thus, as disclosed herein, a growth matrix of the present disclosure, comprising an inoculated substrate or a colonized substrate, can be incubated in a growth environment to produce extra-particle aerial mycelial growth therefrom.

“Inoculated substrate” as used herein refers to a substrate that has been inoculated with fungal inoculum. For example, an inoculated substrate can be formed by combining an uninoculated substrate with a fungal inoculum. An inoculated substrate can be formed by combining an uninoculated substrate with a previously inoculated substrate. An inoculated substrate can be formed by combining an inoculated substrate with a colonized substrate.

“Spawn” as used herein refers to the carrier of a living fungal culture of mycelium grown onto or within a substrate and held in stasis until it is ready to transfer into another substrate for growth. Spawn is formed by inoculating a substrate with fungal inoculum and incubating for sufficient time to allow for fungal colonization. Spawn can be made with, for example, soy, wood, or seeds including white millet, and can be in the form of shavings, pellets, chips, flakes, flour, particles, liquids, or combinations thereof, and is not intended to be limiting to a particular size, shape, or state of matter. For example, spawn can be implemented in a liquid or solid form.

“Casing layer” as used herein refers to a layer of organic or inorganic material placed on top of or below a patterning of spawn. A casing layer can be made of, for example, vermiculite, peat moss, coconut coir, any material that can be used as an uninoculated substrate, or a combination of such materials. A casing layer can be included above or below a patterning of spawn or both above and below a patterning of spawn. In some embodiments a casing layer can serve as a means for controlling the topology of an aerial mycelium.

“Colonized substrate” as used herein refers to an inoculated substrate that has been incubated for sufficient time to allow for fungal colonization. A colonized substrate of the present disclosure can be characterized as a contiguous hyphal mass grown throughout the entirety of the volume of the growth media substrate. The colonized substrate may further contain residual nutrition that has not been consumed by the colonizing fungus. As is understood by persons of ordinary skill in the art, a colonized substrate has undergone primary myceliation, sometimes referred to by skilled artisans as having undergone a “mycelium run.” Thus, in some particular aspects, a colonized substrate consists essentially of a substrate and a colonizing fungus in a primary myceliation phase. For many fungal species, asexual sporulation occurs as part of normal vegetative growth, and as such could occur during the colonization process. Non-limiting examples of such fungi include Ganoderma, Grifola, Laetiporus, Polyporus, Cerioporus, Laricifomes, Fomes and Fomitopsis. Accordingly, in some embodiments, a colonized substrate of the present disclosure may also contain asexual spores (conidia). In some aspects, a colonized substrate of the present disclosure can exclude growth progression into sexual reproduction and/or vegetative foraging. Sexual reproduction includes fruiting body formation (primordiation and differentiation) and sexual sporulation (meiotic sporulation). Vegetative foraging includes any mycelial growth away from the colonizing substrate (such as aerial growth). Thus, in some further aspects, a colonized substrate can exclude mycelium that is in a vertical expansion phase of growth. As used herein, a vertical expansion phase occurs between primary myceliation and primordiation. A colonized substrate can enter a mycelial vertical expansion phase during incubation in a growth environment of the present disclosure. For example, a colonized substrate can enter a mycelial vertical expansion phase upon introducing aqueous mist into the growth environment and/or depositing aqueous mist onto colonized substrate and/or any ensuing extra-particle growth. In some embodiments, the use of aqueous mist can be adjusted, for example, to desired levels and timing, to affect the topology of the growth.

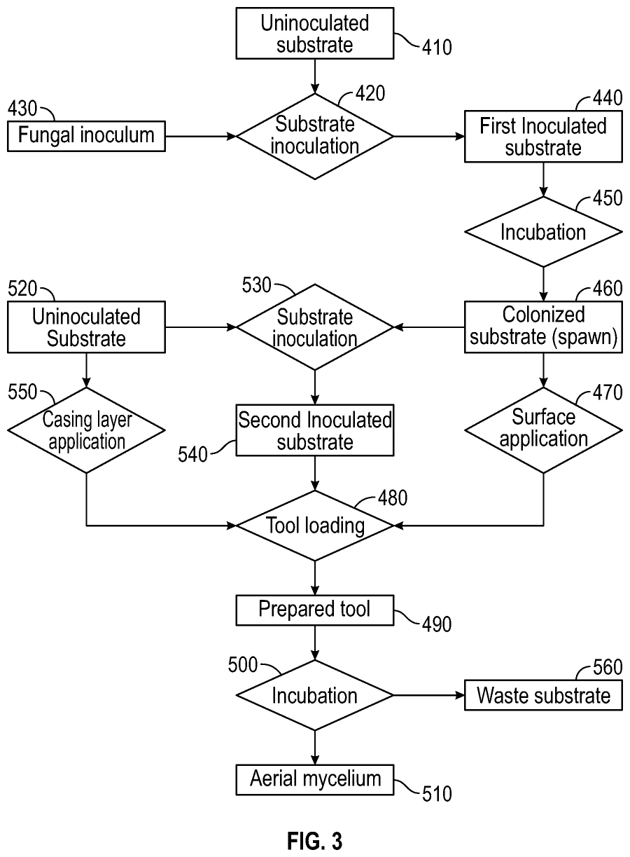

To better understand these terms, a flow chart of an embodiment of the present disclosure is illustrated in FIG. 3. An uninoculated substrate 410 may be inoculated 420 with a fungal inoculum 430 to form a first inoculated substrate 440. After a period of time of incubation 450, the first inoculated substrate 440 can become a colonized substrate which may be referred to as spawn 460. In some embodiments, spawn 460 may be applied 470 to the surface of a substrate which has been or may subsequently be loaded 480 into a tool (which can, for example, comprise a tray or a carrier surface) to form a prepared tool 490. The prepared tool 490 may be incubated 500 for aerial mycelial growth 510. In some embodiments, spawn 460 may be combined with uninoculated substrate 520 to serve in the process of substrate inoculation 530 to form a second inoculated substrate 540 before further incubation 500. A tool 490 may be prepared with or without a casing layer application 550 and with or without a surface application 470 of spawn 460 before a period of time of incubation 500. After a period of time, the aerial mycelium 510 may be separated from the waste substrate 560 (which can comprise waste colonized substrate) and may be further processed.

In some aspects, the present disclosure provides for a method of preparing a growth media or a growth matrix. A prepared and unsterilized substrate is an available resource with high and unselective inoculum potential. Reducing the bioburden of the substrate prior to inoculation with a target fungus can minimize or exclude other potential colonizers. Thus, in some aspects, growth media of the present disclosure can be treated to reduce its bioburden prior to inoculation with the target fungus. In a non-limiting example, the growth media bioburden can be reduced by pasteurization or sterilization, including heat sterilization or steam sterilization of the growth media, each of which may include pressure. In some embodiments, the growth media bioburden can be reduced by irradiation with electromagnetic radiation. In some embodiments, the electromagnetic radiation comprises gamma rays, X-rays, UV, or UV-visible radiation. In some embodiments, the growth media bioburden can be reduced by plasma sterilization. In some embodiments, the growth media bioburden can be reduced by chemical treatment. In some embodiments, the growth media bioburden can be reduced by treatment with ethylene oxide. In some embodiments, the growth media bioburden can be reduced by treatment with hydrogen peroxide. The hydrogen peroxide treatment can include exposure of the substrate (or growth media) to hydrogen peroxide vapor, hydrogen peroxide solution, or both. In some embodiments, the growth media bioburden can be reduced by treatment with alkali. In a non-limiting example, a substrate (or growth media) can be treated by exposure to an alkaline solution, including but not limited to soaking the substrate (or growth media) in an alkaline solution.

By reducing the bioburden of the substrate (or growth media) to minimize or exclude all potential colonizers prior to inoculation, and then inoculating the resulting substrate (or growth media) with the target fungus at a suitable inoculation rate, and physically excluding competitors (e.g., by containing the inoculated substrate or growth media in a sealed bag, colonizing in a sterile or sanitary environment, etc.), a high and selective inoculum potential is created for the target fungus. Colonization of the substrate by the target fungus creates a priority effect for the target fungus based on spatial, metabolic, and chemical dominance (spanning both the intra- and inter-particle matrix comprising the substrate and colonizing fungus), offsetting the subsequent need for physical exclusion of competitors. In general, colonization shifts the exclusion of competitors/contaminants from a physical system level to a biological level and increases freedom to operate on the physical system level. Once this shift in fungal dominance over competitors has occurred, if the inter-particle matrix is broken up or fragmented into discrete particles, the fungus still has a functional priority effect; that is, each discrete particle is still spatially and metabolically dominated by the target fungus.

Thus, in some embodiments, a colonized substrate can be fragmented into smaller portions to provide a fragmented colonized substrate. As used herein, “fragmented colonized substrate” refers to a plurality of discrete particles of colonized substrate. The discrete colonized substrate particles can be characterized as having a particle size. The particle size can have a range, wherein the maximum particle size is less than that of the colonized substrate prior to the fragmentation, and the minimum particle size is substantially the same as the particle size of the substrate prior to the colonization. Methods of fragmenting the colonized substrate can include applying sufficient force to the colonized substrate such that the colonized substrate is fragmented into a plurality of discrete colonized substrate particles. This may simply involve breaking up the colonized substrate into “clumps.” The fragmentation can be performed on a colonized substrate contained in a container. For example, the container can be an aerated bag within which a substrate underwent colonization. Force can be applied to the contained colonized substrate to provide a contained fragmented substrate. Alternatively, the fragmentation can be performed after removal of the colonized substrate from a container. For example, a colonized substrate can reside on an open tray or surface and be physically fragmented, e.g., by hand, machine, or other means of applying force.

Any suitable substrate can be used alone, or optionally combined with a nutrient source, as media to support mycelial growth. The growth media can be hydrated to a final target moisture content prior to inoculation with a fungal inoculum. In a non-limiting example, the substrate or growth media can be hydrated to a final moisture content of at least about 50% (w/w), at most about 80% w/w, within a range of about 50% (w/w) to about 80% (w/w), about 50% (w/w) to about 75% (w/w), within a range of about 50% (w/w) to about 65% (w/w), within a range of about 50% (w/w) to about 60% (w/w), or within a range of about 60% (w/w) to about 70% (w/w). Growth media hydration can be achieved via the addition of any suitable source of moisture. In a non-limiting example, the moisture source can be liquid phase water, an aqueous solution containing one or more additives (including but not limited to a nutrient source), and/or gas phase water. In some embodiments, at least a portion of the moisture is derived from steam utilized during bioburden reduction of the growth media. In some embodiments, inoculation of the growth media with the fungal inoculum can include a further hydration step to achieve a target moisture content, which can be the same or different than the moisture content of the growth media. For example, if growth media loses moisture during fungal inoculation, the fungal inoculated growth media can be hydrated to compensate for the lost moisture.

In some embodiments, a growth matrix of the present disclosure is provided as a solid-state matrix. In other embodiments, a growth matrix of the present disclosure is provided as a slurry.

Methods for the production of aerial mycelium disclosed herein require an inoculation stage, wherein an inoculum is used to transport an organism into a substrate. The inoculum, which carries a desired fungal strain, is produced in sufficient quantities to inoculate a target quantity of substrate. The inoculation can provide a plurality of myceliation sites (nucleation points) distributed throughout the substrate. Inoculum can take the form of a liquid, a slurry, or a solid, or any other known vehicle for transporting an organism from one growth-supporting environment to another. Generally, the inoculum comprises water, carbohydrates, sugars, vitamins, other nutrients, and fungi. The inoculum typically contains enzymatically available carbon and nitrogen sources (e.g., lignocellulosic biomass, chitinous biomass, carbohydrates) augmented with additional micronutrients (e.g., vitamins, minerals). The inoculum can contain inert materials (e.g., perlite). In a non-limiting example, the fungal inoculum can be a seed-supported fungal inoculum, a feed-grain-supported fungal inoculum, a seed-sawdust mixture fungal inoculum, or another commercially available fungal inoculum, including specialty proprietary spawn types provided by inoculum retailers. In some aspects, a fungal inoculum can be characterized by its density. “Feed-grain” herein refers to grain used for agriculture or food, as distinguished from “growth-grain” as defined and used elsewhere herein. In some embodiments, a fungal inoculum has a density of about 0.1 gram per cubic inch to about 10 grams per cubic inch, or from about 1 gram per cubic inch to about 7 grams per cubic inch. A skilled person can modify variables including the substrate or growth media component identities, substrate or growth media nutrition profile, substrate or growth media moisture content, substrate or growth media bioburden, inoculation rate, and inoculum constituent concentrations to arrive at a suitable medium to support aerial mycelial growth. In some embodiments, the inoculation rate can be expressed as a percentage of the target volume of the substrate or growth media (% (v/v)). In some embodiments, the inoculation rate can range from about 0.1% (v/v) to about 80% (v/v). In some embodiments, the inoculation rate is at most about 50% (v/v), at most about 45% (v/v), at most about 40% (v/v), at most about 30% (v/v), at most about 25% (v/v), at most about 20% (v/v), at most about 15% (v/v), at most about 10% (v/v) or at most about 5% (v/v). In some embodiments, the inoculation rate is about 1% (v/v), about 2% (v/v), about 3% (v/v), about 4% (v/v), about 5% (v/v), about 6% (v/v), about 7% (v/v), about 8% (v/v), about 9% (v/v), about 10% (v/v), about 11% (v/v), about 12% (v/v), about 13% (v/v), about 14% (v/v), about 15% (v/v), about 16% (v/v), about 17% (v/v), about 18% (v/v), about 19% (v/v), about 20% (v/v), about 21% (v/v), about 22% (v/v), about 23% (v/v), about 24% (v/v), about 25% (v/v), about 26% (v/v), about 27% (v/v), about 28% (v/v), about 29% (v/v) or about 30% (v/v); or any range therebetween. In some embodiments, the inoculation rate can be expressed as a percentage of the target dry mass of the substrate or growth media (% (w/w)). In some embodiments, the inoculation rate can range from about 0.1% (w/w) to about 80% (w/w). In some embodiments, the inoculation rate is at most about 50% (w/w), at most about 45% (w/w), at most about 40% (w/w), at most about 30% (w/w), at most about 25% (w/w), at most about 20% (w/w), at most about 15% (w/w), at most about 10% (w/w) or at most about 5% (w/w). In some embodiments, the inoculation rate is about 1% (w/w), about 2% (w/w), about 3% (w/), about 4% (w/w), about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w), about 20% (w/w), about 21% (w/w), about 22% (w/w), about 23% (w/w), about 24% (w/w), about 25% (w/w), about 26% (w/w), about 27% (w/w), about 28% (w/w), about 29% (w/w) or about 30% (w/w); or any range therebetween.

As disclosed herein, a growth medium of the present disclosure can be inoculated after reducing its bioburden. When the method of reducing the growth media bioburden involves heat, it can be necessary to cool the growth media prior to adding the fungal inoculum to maintain fungal viability for subsequent growth. In a non-limiting example, a growth medium can be steam sterilized and subsequently cooled to ambient room temperature, or to no greater than about 37° C. In another non-limiting example, the growth medium can be cooled to fall within a temperature range suitable to support fungal mycelial growth, such as a temperature that supports primary myceliation when the inoculated growth media is intended for subsequent incubation under colonization conditions to produce a colonized substrate, or to a temperature that supports extra-particle aerial mycelial growth when the inoculated growth media is intended for subsequent incubation in a growth environment to produce an aerial mycelium.

A growth medium inoculated with fungal inoculum can be incubated in a growth environment of the present disclosure for an incubation time period to produce extra-particle aerial mycelial growth. Alternatively, the fungal-inoculated growth medium can be incubated under colonization conditions in a colonization environment of the present disclosure for a colonization time period to provide a colonized substrate, which is optionally fragmented. The colonized substrate or fragmented colonized substrate can be subsequently incubated in the growth environment for an incubation time period to produce extra-particle aerial mycelial growth.

A colonization environment of the present disclosure refers to an environment that supports primary myceliation. The colonization environment is characterized as having a temperature and a relative humidity that supports primary myceliation. In some embodiments, a colonization environment of the present disclosure can exclude a condition that produces aerial mycelial growth; thus, the colonization environment can exclude aqueous mist. The colonization environment can include a primary colonization environment immediately surrounding the fungal-inoculated growth medium and an optional secondary colonization environment surrounding the primary colonization environment. Typically, the colonization conditions allow for sufficient aeration of the fungal-inoculated growth medium during or throughout the colonization time period. Thus, in some embodiments, the colonization conditions allow for gas exchange to occur between the fungal-inoculated growth medium in a primary colonization environment and the secondary colonization environment. In a non-limiting example, the fungal-inoculated growth medium can be contained in a breathable container characterized as having a primary colonization environment, and the container can be stored in the secondary colonization environment. Non-limiting examples of a breathable container include a perforated bag, a microperforated bag or a gas-permeable filter patch bag; such containers can be otherwise sealed. In some embodiments, the primary colonization environment temperature is within a range of about 4° C. to about 37° C. In some more particular embodiments, the primary colonization environment temperature is within a range of about 15° C. to about 30° C. In some further embodiments, the primary colonization temperature is within a range of about 30° C. to 31° C. The colonization temperature can be optimized for a particular fungus and can be above or below the recited ranges for extremophiles. In some embodiments, the primary colonization environment relative humidity can be at least about 90%, at least about 95%, at least about 96%, at least about 97%, at least about 98%, or at least about 99%. In some embodiments, the fungal-inoculated growth medium is characterized as having colonization pH. In some embodiments, the fungal-inoculated growth medium pH can be at least about pH 3 and at most about pH 10 over the course of the colonization time period or can be at least about pH 3 and at most about pH 7 over the course of the colonization period. In some embodiments, the pH of the fungal-inoculated growth medium is within a range of about pH 6 to about pH 7 at the beginning of the colonization time period and decreases to a pH within a range of about pH 3 to about pH 5 or about pH 4 to about pH 5 over the course of the colonization time period, concurrent with the primary myceliation of the substrate. In some embodiments, the first and second colonization environments are substantially the same, at least with respect to temperature and relative humidity. In some embodiments, the relative humidity of the secondary colonization environment can be substantially lower than that of the primary colonization environment, so long as sufficient relative humidity is maintained in the primary colonization environment (e.g., inside the sealed bag or container).

A fungal-inoculated growth medium can be incubated in a colonization environment for a colonization time period. A colonization time period is sufficient to allow the substrate to be fully colonized, thereby producing a single colony of mycelium containing the substrate, which typically presents as a contiguous matrix. In some embodiments, the colonization time period can end no later than when a visible fruiting body forms, but most preferably prior to when a visible fruiting body forms. In a non-limiting example, the colonization time period can end prior to a karyogamy or meiosis phase of the fungal reproductive cycle. In some embodiments, the substrate colonization time period is within a range of about 1 day to about 3 weeks and can optimized based on the target fungus. In some embodiments, the substrate colonization time period is about 1 day to about 10 days. Trial substrate colonization runs can be used to inform the period of time in the colonization environment during which substrate colonization is achieved without the formation of visible fruiting bodies.

After incubation of the fungal-inoculated growth medium in the colonization environment for the colonization time period, or prior to incubating a colonized substrate to a growth environment, supplemental nutrition and/or moisture can be added to the colonized substrate. In some embodiments, the supplemental nutrition is a macronutrient. In some embodiments, the macronutrient is provided as a flour. When a colonized substrate is fragmented to provide a fragmented colonized substrate, supplemental nutrition and/or moisture can be added during or after the fragmentation, and prior to incubating the fragmented colonized substrate in the growth environment.

In some further aspects, a colonized substrate can be stored in a storage environment for a storage time period prior to incubating the colonized substrate in a growth environment of the present disclosure. The storage can allow for a colonized or fragmented colonized substrate to be stockpiled for subsequent use or for other logistical purposes. The storage environment and storage time period are typically adjusted such that the colonized or fragmented colonized substrate, at the end of the storage time period, is substantially similar to the colonized or fragmented colonized substrate at the end of the colonization time period. Thus, the storage environment can be an environment that slows the metabolism of the colonizing fungus and/or maintains the colonizing fungus in a primary myceliation stage. In a non-limiting example, the storage environment can be an environment having a temperature substantially below that of the colonization environment. In another non-limiting example, the storage environment can be a refrigerated environment. In some further embodiments, the storage time in the colonization environment can be up to about a month.

In some aspects, the present disclosure provides for growth media preparation, fungal-inoculated growth medium preparation, and subsequent mycelial growth to occur in a single locale, or in two or more locales. For example, growth media bioburden reduction and/or inoculation can be performed in a locale that is the same or different from the locale wherein substrate colonization and/or aerial mycelial growth occurs. Methods of reducing growth media bioburden, including those disclosed herein (supra) or generally known in the art, can be applied to “in situ” bioburden reduction of a locale of the present disclosure, including a first, second or third (or further) locale, as disclosed herein, and the contents thereof. A means and/or agent suitable for use in reducing bioburden of a locale can be introduced to the locale via one or more points of entry to the locale, including an HVAC system, a misting system, a 3D printer, and the like.

In some embodiments, reduction of growth media bioburden occurs in a first locale, inoculation of the resulting growth media with fungal inoculum occurs a second locale, and production of mycelial growth (e.g., substrate colonization and/or extra-particle aerial mycelial growth from the fungal-inoculated growth medium) occurs in a third locale.

In other embodiments, reduction of growth media bioburden occurs in a first locale, inoculation of the resulting growth media with fungal inoculum occurs in the first locale, and production of mycelial growth (e.g., substrate colonization and/or extra-particle aerial mycelial growth from the resulting fungal-inoculated growth medium) occurs in a second locale.

In yet other embodiments, reduction of growth media bioburden occurs in a first locale, inoculation of the resulting growth media with fungal inoculum occurs in the first locale, and production of mycelial growth (e.g., substrate colonization and/or extra-particle aerial mycelial growth from the resulting fungal-inoculated growth medium) also occurs in the first locale.

In some more particular embodiments, the growth media is loaded into a growth locale. The growth locale can be a growth chamber or system configured to support conditions required for each of growth media bioburden reduction, growth media inoculation, and the growth of a mycelium of the present disclosure. The growth media within the growth locale is treated to reduce the growth media bioburden to the desired level, and subsequently inoculated with fungal inoculum therein. The growth locale conditions can be subsequently modified to a growth environment of the present disclosure, with an optional interim substrate colonization step. In a non-limiting example, the locale is a growth chamber equipped with hardware that supports the growth media (e.g., trays, conveyer belts, shelves, beds or other surfaces or containers, as disclosed herein), and the growth media is loaded into the growth chamber. This can be achieved at scale using a conventional head filler, conveyer, or other loading equipment. If necessary, the growth chamber is closed off from the ambient environment in preparation for the bioburden reduction step. The growth media is then treated (e.g., with steam) to achieve the desired level of sterility. The growth chamber environmental conditions are then modified to those that support inoculation, and the resulting growth media is then inoculated “in place” by any suitable means. For example, an automated robotic system can be used to inoculate the growth media. In some embodiments, the fungal inoculum can be in the form of a liquid or a slurry. Accordingly, the liquid or slurry inoculum could be pumped into the chamber and deposited into or onto the growth media, e.g., via a sprayer, a 3D printer, or other suitable means. The growth chamber environmental conditions are then modified to those that support mycelial growth. The resulting fungal-inoculated growth medium is then exposed to the growth environment conditions to produce the mycelial growth. Optionally, the modification of the growth locale conditions to a growth environment is preceded by modification of the growth locale conditions to those of a colonization environment. Thus, each step of bioburden reduction, inoculation, optionally, substrate colonization, and aerial mycelial growth production can be performed in a single locale.

### Definitions and Methods Related to Growth Environment

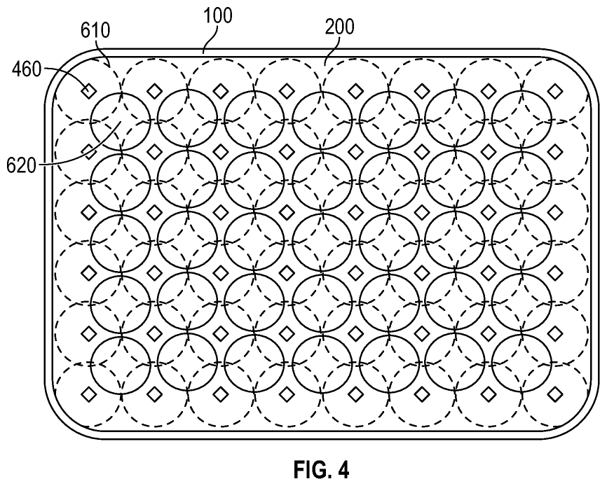

FIG. 4 illustrates an embodiment of the present disclosure, showing a pattern of aerial mycelial growth resulting from a method to affect the topology thereof. The pattern can result when spawn is applied to a substrate in a geometrically regular pattern, resulting in aerial mycelial growth with an expression of bulbous features (previously shown as 331 in FIG. 2) in a defined geometry. In this figure, a packed tool (e.g., tray, container, belt, carrier sheet, etc.) 100 contains a substrate 200. Spawn 460 is applied to the substrate 200 in the packed tool 100 at various distinct positions (e.g., in a grid pattern, with various numbers of positions (e.g., numbering forty-eight (48) in FIG. 4). The spawn can be applied in other desired patterned configurations, such as concentric perimeters (e.g., concentric rings), a spiral shape, rows (e.g., rows of continuous lines of spawn); columns (e.g., columns of continuous lines of spawn), a spoked pattern, and other suitable regular or irregular shapes). After incubation starts (e.g., in 2 days), radial growth 610 can be visible extending from the spawn 460. Radial growth 610 continues until reaching a stage at which the margins of discrete radial colonies intersect one another. As incubation continues (e.g., from approximately days 3-5), discrete bulbous aerial formations 620 (formed by the intersecting radial growth 610) initiate at positions between areas where spawn 460 had been applied, resulting in a regular pattern of bulbous expression over the surface of the substrate. Aerial expansion has been observed as these bulbous formations between areas of spawn application, with a lack of aerial growth extending directly from the areas where spawn had been applied. Various methods disclosed herein can be used to grow aerial mycelium with geometric regularity or with a homogeneous growth expression of a lack of distinct bulbous features. The remaining figures and additional embodiments are provided further below.

US Published Patent Application 2015/0033620, the entire contents of which is hereby incorporated by reference in its entirety to the extent not inconsistent with the content of this disclosure, describes techniques for growing a material comprising aerial mycelium, referred to as a “mycological biopolymer.” As described therein, a mycological biopolymer product provided by the disclosed method is characterized as containing a homogeneous biopolymer matrix that is comprised predominantly of fungal chitin and trace residues (e.g., beta-glucan, proteins). The mycological biopolymer is up-cycled from domestic agricultural lignocellulosic waste and is made by inoculating the substrate made of domestic agricultural lignocellulosic waste with a selected fungus in a container that is sealed off from the ambient environment external to the container. In addition to the substrate and fungal inoculum, the container contains a void space. A network of undifferentiated aerial mycelium comprising a chitin-polymer grows into and fills the void space of the container. The chitin-polymer-based aerial mycelium is subsequently extracted from the substrate and dried. As further described in US2015/0033620, the environmental conditions for producing the mycological biopolymer product described therein, i.e., a high carbon dioxide (CO₂) content (about 3% to about 7% by volume) and an elevated temperature (from about 85° F. to about 95° F.), prevent full differentiation of the fungus into a mushroom, as evidenced by the absence of a visible fruiting body.

In one aspect, the present disclosure provides an aerial mycelium. In a further aspect, the aerial mycelium does not contain a visible fruiting body.

As described in WO2019/099474A1, the entire contents of which is hereby incorporated by reference in its entirety to the extent not inconsistent with the content of this disclosure, another method of growing a mycological biopolymer material employs incubation of a substrate with nutritive value inoculated with a fungus in containers that are placed in a closed incubation chamber with air flows passed over each container while the chamber is maintained with a predetermined environment of humidity, temperature, carbon dioxide, and oxygen.

The aerial mycelia of the present disclosure are growth products obtained from an inoculated substrate incubated for a period of time (i.e., an incubation time period) in a growth environment, as disclosed herein.

In some aspects, a method of making an aerial mycelium of the present disclosure comprises placing a growth matrix in contact with a tool. In some aspects, the tool can have a base having a surface area. In some embodiments, the surface area can be at least about 1 square inch. In some embodiments, the surface area can be at most about 2000 square feet. In some embodiments, the growth matrix can be placed in contact with the base, e.g., placed on top of or distributed across the base. In some embodiments, the base can be a planar surface. Non-limiting examples of a tool include a tray, a sheet, a table, or a conveyer belt. In some embodiments, the tool can have at least one wall. In some embodiments, the base and the at least one wall can together form a cavity. In some embodiments, the growth matrix can be placed or packed in a tool having a cavity. In some embodiments, the tool can be an uncovered tool. In some other embodiments, the tool can have a lid, the lid having at least one opening, or the tool can be covered at least in part with a perforated barrier. Non-limiting embodiments of a tool having a lid with an opening are disclosed in US2015/0033620A1. An uncovered tool, or a tool having a lid with an opening or a perforated barrier, and further having growth matrix on or within the tool, can allow for aqueous mist to be deposited onto the growth matrix surface, and/or onto any resulting mycelial growth.

“Growth environment” as used herein refers to an environment that supports the growth of mycelia, as would be readily understood by a person of ordinary skill in the art in the mycelial cultivation industry, which contains a growth atmosphere having a gaseous environment of carbon dioxide (CO₂), oxygen (O₂), and a balance of other atmospheric gases including nitrogen (N₂), and which is further characterized as having a relative humidity. In some aspects of the present disclosure, the growth atmosphere can have a CO₂ content of at least about 0.02% (v/v), at least about 0.6%, at least about 5% (v/v), less than about 10% (v/v), less than about 8% (v/v), less than about 7%, between about 0.02% and 10%, between about 0.02% and 8%, between about 0.6% and about 7%, between about 5% and about 10%, or between about 5% and about 8%. In some other aspects, the growth atmosphere can have an O₂ content of at least about 12% (v/v), or at least about 14% (v/v), and at most about 21% (v/v). In yet other aspects, the growth atmosphere can have an N₂ content of at most about 79% (v/v). Each foregoing CO₂, O₂ or N₂ content is based on a dry gaseous environment, notwithstanding the growth environment atmosphere relative humidity.

In some other aspects of the present disclosure, there is provided a method of making an aerial mycelium comprising incubating a growth matrix in a growth environment for an incubation time period, wherein the growth matrix comprises a substrate and a fungus; introducing aqueous mist into the growth environment throughout the incubation time period or a portion thereof; and producing extra-particle aerial mycelial growth from the growth matrix; wherein introducing comprises introducing aqueous mist into the growth environment resulting in a mean mist deposition rate that is below about 0.01 uL/cm²/hour. In some embodiments, introducing comprises introducing aqueous mist into the growth environment resulting in a mean mist deposition rate that does not result in a detectable quantity of deposited mist in the growth environment. In some embodiments, introducing comprises introducing aqueous mist into the growth environment resulting in a mean mist deposition rate that does not result in a visible quantity of deposited mist in the growth environment. In some embodiments, introducing comprises introducing aqueous mist into the growth environment resulting in a mean mist deposition rate that does not result in a measurable quantity of deposited mist in the growth environment. In some embodiments, introducing comprises introducing aqueous mist into the growth environment resulting in a mean mist deposition rate that does not result in detectable quantities of deposited mist in the growth environment on the growth matrix, the extra-particle aerial mycelial growth, or both. In some embodiments, the growth matrix comprises a substrate and a fungal inoculum, said fungal inoculum comprising a fungus. In some other embodiments, the growth matrix comprises a colonized substrate, said colonized substrate comprising a substrate, wherein said substrate is previously colonized with mycelium of a fungus. In some embodiments, the colonized substrate is a fragmented colonized substrate.

In some other aspects of the present disclosure, there is provided a method of making an aerial mycelium comprising incubating a growth matrix in a growth environment for an incubation time period, wherein the growth matrix comprises a substrate and a fungus; introducing aqueous mist into the growth environment throughout the incubation time period or a portion thereof; and producing extra-particle aerial mycelial growth from the growth matrix; wherein the total quantity of aqueous mist resulting from the introducing aqueous mist that is deposited on the growth matrix, the resulting extra-particle aerial mycelial growth, or both, is negligible. In some embodiments, the growth matrix comprises a substrate and a fungal inoculum, said fungal inoculum comprising a fungus. In some other embodiments, the growth matrix comprises a colonized substrate, said colonized substrate comprising a substrate, wherein said substrate is previously colonized with mycelium of a fungus. In some embodiments, the colonized substrate is a fragmented colonized substrate.

In some other aspects of the present disclosure, there is provided a method of making an aerial mycelium comprising providing a growth environment, the growth environment comprising an amount of aqueous mist; incubating a growth matrix in the growth environment for an incubation time period, wherein the growth matrix comprises a substrate and a fungus, wherein incubating comprises exposing the growth matrix to aqueous mist during at least a portion of the incubation time period; and producing extra-particle aerial mycelial growth from the growth matrix; wherein a mean mist deposition rate resulting from the amount of aqueous mist during the at least a portion of the incubation time period is below about 0.01 uL/cm²/hour. In some embodiments, the mean mist deposition rate is below an amount that results in a detectable quantity of deposited mist in the growth environment. In some embodiments, the method further comprises introducing aqueous mist into the growth environment. In some embodiments, the growth matrix comprises a substrate and a fungal inoculum, said fungal inoculum comprising a fungus. In some other embodiments, the growth matrix comprises a colonized substrate, said colonized substrate comprising a substrate, wherein said substrate is previously colonized with mycelium of a fungus. In some embodiments, the colonized substrate is a fragmented colonized substrate.

Temperature and Light

In some further aspects, a method of making an aerial mycelium of the present disclosure comprises incubating the growth matrix in a growth environment, wherein the growth environment has a temperature that supports mycelial growth. In some embodiments, the growth environment has a temperature within a range of about 55° F. to about 100° F., or within a range of about 60° F. to about 95° F. In some more particular embodiments, the growth environment has a temperature within a range of about 80° F. to about 95° F., or within a range of about 85° F. to about 90° F. throughout the incubation time period. In other embodiments, the growth environment has a temperature within a range of about 60° F. to about 75° F., within a range of about 65° F. to about 75° F., or within a range of about 65° F. to about 70° F. In some embodiments, the growth environment temperature can be tuned to optimize for the growth of a particular fungal genus, species, or strain. In some further embodiments, the growth environment temperature can be tuned to improve the homogeneity of growth topology of a particular fungal genus, species, or strain. In some particular embodiments, the growth environment has a temperature within a range of about 86° F. to 88° F.

In some aspects of the present disclosure, the growth environment suitable for the growth of the aerial mycelia of the present disclosure can be a dark environment. “Dark environment” as used herein in connection with a growth environment would be readily understood by a person of ordinary skill in the art in the mycelial cultivation industry and refers to an environment without natural or ambient light and without growing lights.

Exposing fungi to white light, and especially blue light, has been associated with the induction of fruiting and the enhancement of production efficiency of oyster mushrooms (e.g., see Roshita & Goh, AIP Conference Proceedings 2030, 020110 (2018)), the entire contents of which are hereby incorporated by reference in their entirety to the extent not inconsistent with the content of this disclosure). Surprisingly, Applicant has discovered that an aerial mycelium of the present disclosure, absent visible fruiting bodies, can be prepared by the methods of the present disclosure in the presence of white light, which includes blue light. Aerial mycelium prepared in the presence of white light was consistent in yield, thickness, density, morphology and in the absence of visible fruiting bodies when compared to control aerial mycelia produced under the same growth conditions but in a dark environment. Thus, in some embodiments, a growth environment suitable for the growth of the aerial mycelia of the present disclosure is not a dark environment. In some embodiments, the growth environment does not exclude light. In some embodiments, the growth environment can include natural light. In some embodiments, the growth environment can include ambient light. In some embodiments, the growth environment can include a growing light.

Air Content and Air Flow

As disclosed in US2015/0033620, environmental conditions for producing a mycological biopolymer include a CO₂ content of about 3% to about 7% (v/v) to prevent full differentiation of the fungus into a mushroom. Accordingly, in some aspects, the present disclosure provides for methods of producing an aerial mycelium in a growth environment comprising a growth atmosphere, wherein the growth atmosphere can have a CO₂ content within a range of about 3% (v/v) to about 7% (v/v), or within a range of about 5% (v/v) to about 7% (v/v). In some embodiments, the growth atmosphere can have a CO₂ content of about 3%, about 4%, about 5%, about 6%, or about 7% (v/v), or any range therebetween.

Surprisingly, Applicant has discovered that an aerial mycelium of the present disclosure can be produced without visible fruiting bodies under conditions wherein aqueous mist is introduced into a growth environment having a growth atmosphere containing much lower CO₂ content. For example, Applicant has shown that aerial mycelia obtained from a growth environment of circulating mist and an atmosphere having a mean CO₂ content of about 0.04% (v/v) over the course of the incubation time period or having a mean CO₂ content of about 2% (v/v) over the incubation time period were similar in yield, thickness, density, and morphology to aerial mycelia obtained via growth in an atmosphere having a mean CO₂ content of 5% (v/v) but otherwise identical growth conditions. Thus, the present disclosure advantageously provides for a safer, more efficient, and more cost-effective manufacturing process with reduced environmental impact (e.g., by circumventing the risk of exposure to high CO₂ content growth environments, increasing operator accessibility to growth environments, eliminating the costs associated with CO₂ injection into the growth environment, and reducing off-gassing of CO₂ into the atmosphere). As further disclosed herein, Applicant has shown that aerial mycelia of increased thickness can be obtained via incubation in a growth environment characterized as having a particular misting profile. Prior to this discovery, efforts to obtain thicker aerial mycelia included extending incubation time periods to support continued aerial growth over time. As extended incubation time periods can increase the risk of fruiting body formation, strategies known to attenuate fruiting body formation (e.g., elevated CO₂ content) were simultaneously employed. The present disclosure advantageously provides for methods of making aerial mycelia of increased thickness, absent visible fruiting bodies, by adopting preselected misting profiles, without requiring a high CO₂ content growth environment. The ability to increase aerial mycelial thickness, absent visible fruiting bodies, by tuning mist deposition rate can also advantageously reduce incubation time periods, thereby allowing more efficient production of aerial mycelia and reduced risk of microbial contamination that can occur in high moisture environments.

Thus, the present disclosure provides for a method of growing aerial mycelia in a growth environment comprising a growth atmosphere having markedly reduced CO₂ content compared to the prior state of the art of growing aerial mycelia. Accordingly, in some embodiments, the growth atmosphere CO₂ content can be less than about 3% (v/v). In some embodiments, the growth atmosphere CO₂ content can be no greater than about 2.9% (v/v), no greater than about 2.8% (v/v), no greater than about 2.7% (v/v), no greater than about 2.6% (v/v) or no greater than about 2.5% (v/v). In some further embodiments, the growth atmosphere CO₂ content can be less than 2.5% (v/v). In some embodiments, a growth atmosphere of the present disclosure can have a CO₂ content of at least about 0.02% (v/v). In some embodiments, a growth atmosphere of the present disclosure can have a CO₂ content of at least about 0.03% (v/v). In some further embodiments, the growth atmosphere CO₂ content can approximate ambient atmospheric CO₂ content; for example, the growth atmosphere CO₂ content can be at least about 0.04% (v/v). In some more particular embodiments, the growth atmosphere CO₂ content can be within a range of about 0.02% to about 3% (v/v), about 0.02 to about 2.5% (v/v), about 0.03% to about 3% (v/v), about 0.03% to about 2.5% (v/v), about 0.04% to about 3% (v/v), or about 0.04% to about 2.5% (v/v).

In other embodiments, the growth atmosphere CO₂ content can be within a wider range. Thus, in some embodiments, the growth atmosphere CO₂ content can be within a range of about 0.02% to about 7% (v/v), within a range of about 0.04% to about 7% (v/v), within a range of about 0.1% to about 7% (v/v), within a range of about 0.2% to about 7% (v/v), within a range of about 1% to about 7% (v/v), or within a range of about 2% to about 7% (v/v); or can be within a range of about 0.02% to about 5% (v/v), within a range of about 0.04% to about 5% (v/v), within a range of about 0.1% to about 5% (v/v), within a range of about 0.2% to about 5% (v/v), or within a range of about 1% to about 5% (v/v). In some more particular embodiments, the growth atmosphere CO₂ content can be about 1%, about 2% or about 3%, or any range therebetween. In yet other embodiments, the growth atmosphere CO₂ content can be a mean CO₂ content over the course of the incubation time period. In some embodiments, the growth atmosphere mean CO₂ content can be less than about 3% (v/v), can be less than 2.5% (v/v), or can be no greater than about 2% (v/v) over the course of the incubation time period.

It is understood that fungal growth requires respiration, which can increase CO₂ content and decrease oxygen (O₂) content in the growth atmosphere, particularly in an enclosed growth environment such as an incubation chamber or “growth chamber.” In some aspects, the present disclosure provides for a growth environment having a growth atmosphere that is maintained during the incubation time period by replenishing the growth environment with one or more of the atmospheric gases, such as CO₂, replenishing the growth environment with air having the same composition as the target growth atmosphere composition, venting the growth environment to reduce content of one or more gases, or a combination thereof. In a non-limiting example, if the CO₂ content in a growth chamber is below a target set point, CO₂ gas can be infused into the growth chamber. Conversely, if the CO₂ content exceeds a target set point, then fresh air having the target growth atmosphere composition can be introduced into the growth chamber while venting the chamber to release the existing air having the high CO₂ content. Accordingly, growth chamber atmospheric content can be maintained via CO₂ and fresh air infusion to maintain a target CO₂ set point; as such, O₂ and other atmospheric components are maintained indirectly and fluctuate as a function of fungal respiration. In some other aspects, the present disclosure provides for a growth environment wherein the growth atmosphere CO₂ and O₂ contents are allowed to modulate with fungal respiration, without adjusting the growth atmosphere to maintain preselected CO₂ or O₂ content. Thus, the growth environment can be a closed system. The present disclosure also provides for a growth environment wherein the growth atmosphere CO₂ and O₂ contents are allowed to modulate with fungal respiration, and further allowing for adjustments to be made to the growth atmosphere under conditions wherein a particular preselected growth atmospheric condition is breached. In a non-limiting example, an aerial mycelium can be grown in a growth atmosphere that allows for natural fungal respiration to occur, with a preselected CO₂ content ranging from about 0.02% to about 7% CO₂ (v/v), wherein the CO₂ content is adjusted (e.g., by injection of CO₂ into the growth atmosphere) if the CO₂ content falls outside the scope of the preselected range.

A growth environment of the present disclosure can be further characterized as having an atmosphere having a pressure as would be readily understood by a person of ordinary skill in the art in the mycelial cultivation industry. In a non-limiting embodiment, a growth atmosphere of the present disclosure can have an atmospheric pressure within a range of about 27 to about 31 inches of mercury (Hg), can have an atmospheric pressure of about 29 to about 31 inches Hg, or can have an atmospheric pressure of about 29.9 inches Hg. In some embodiments, a growth environment of the present disclosure can be characterized as having an ambient atmospheric pressure.

In some aspects of the present disclosure, the growth environment suitable for the growth of the aerial mycelia of the present disclosure is characterized as having an airflow. In a non-limiting embodiment, the airflow velocity is at least 10 ft/minute and at most 50 ft/minute. In some further aspects, the air composition of the airflow can be substantially the same as the composition of the growth environment atmosphere. In some embodiments, an airflow can be used to direct and/or deposit aqueous mist that is present in the growth environment towards or onto a growth matrix. The skilled person can adopt various means of directing the flows of air, including baffles, perforated barriers, airflow boxes and/or other tools that can be suitably positioned in the growth environment or in relation to tools or beds containing growth matrix in order to achieve the desired outcome, including a substantially homogeneous airflow, with respect to direction and/or velocity, across a plurality of growth matrices in the growth environment, and/or a substantially homogeneous introduction and/or deposition of mist in the growth environment.

“Horizontal airflow” as used herein refers to flows of air directed substantially parallel to the surface of a growth matrix and any subsequent extra-particle mycelial growth.

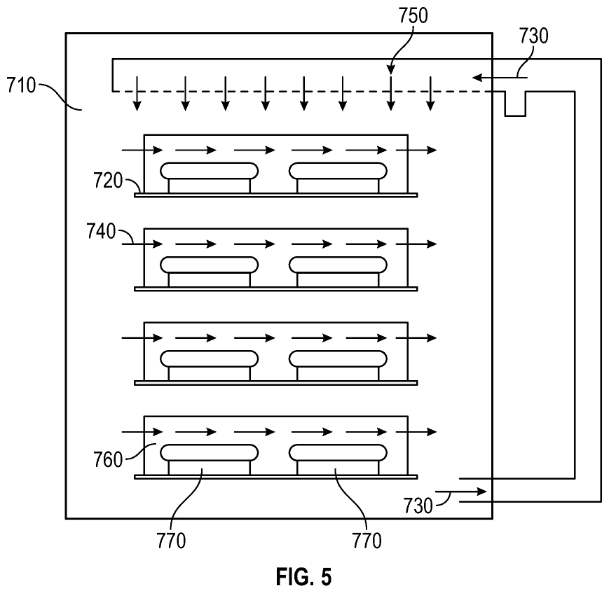

An embodiment of horizontal airflow of the present disclosure is illustrated in FIG. 5. Referring to FIG. 5, a method of growing a mycelium of the present disclosure employs a closed incubation chamber 710 having a plurality of vertically spaced apart shelves 720 and transparent front walls (not shown) for viewing the interior of the chamber 710. In addition, an air flow system 730 is connected with the chamber 710 for directing substantially horizontal air flows across the chamber 710 as indicated by arrows 740 from one side of the chamber 710 to and through the opposite side of the chamber 710. As illustrated, the air flow system 730 includes a manifold 750 in the upper part of the chamber 710 for distributing humidified air across the top of the chamber 710 for cascading down the shelves 720 until being recirculated on the bottom right for re-humidification. Each shelf 720 of the chamber 710 is sized to receive an air box 760 that contains two containers 770, each of which contains growth media comprised of a substrate and a fungus.

Thus, in some other aspects, a method of preparing an aerial mycelium of the present disclosure can include directing an airflow through the growth environment. In some embodiments, the airflow can be a relatively high airflow environment, wherein the airflow can have a velocity of greater than about 250 linear feet per minute (lfm). In other embodiments, the airflow can be a relatively lower airflow environment, wherein the airflow can have a velocity of less than about 150 lfm, less than about 125 lfm, less than about 100 lfm or less than about 75 lfm. In some more particular embodiments, the growth environment can have an airflow, wherein the airflow velocity is less than about 50 lfm, less than about 40 lfm, less than about 30 lfm or less than about 25 lfm.

In some embodiments, the airflow is a substantially horizontal airflow. In some embodiments, the substantially horizontal air flow can have a velocity of no greater than about 350 lfm, or a velocity no greater than about 300 lfm. In other embodiments, the substantially horizontal airflow can have a velocity of no greater than about 275 lfm, a velocity of no greater than about 175 lfm, a velocity of no greater than about 150 lfm, a velocity of no greater than about 125 lfm, or a velocity of no greater than about 110 lfm. In some further embodiments, the velocity is at least about 5 lfm, at least about 10 lfm, at least about 15 lfm, at least about 20 lfm, at least about 25 lfm, at least about 30 lfm, at least about 35 lfm, at least about 40 lfm, at least about 45 lfm or at least about 50 lfm. In some more particular embodiments, the substantially horizontal airflow has mean velocity of about 5 lfm, about 10 lfm, about 15 lfm, about 20 lfm, about 25 lfm, about 30 lfm, about 35 lfm, about 40 lfm, about 45 lfm, about 50 lfm, about 55 lfm, about 60 lfm, about 65 lfm, about 70 lfm, about 75 lfm, about 80 lfm, about 85 lfm, about 90 lfm, about 95 lfm, about 100 lfm, about 105 lfm, about 110 lfm, about 115 lfm or about 120 lfm. In some more particular embodiments still, the substantially horizontal air flow can have a velocity within a range of about 5 lfm to about 125 lfm, within a range of about 5 lfm to about 100 lfm, within a range of about 5 lfm to about 75 lfm, or within a range of about 5 lfm to about 50 lfm. In yet more particular embodiments, the substantially horizontal air flow can have a velocity within a range of about 5 lfm to about 40 lfm, or within a range of about 5 to about 25 lfm. In other embodiments, the substantially horizontal air flow can have a velocity within a range of about 40 lfm to about 120 lfm. Without being bound to any particular theory, the flows of air can facilitate the distribution of mist throughout the growth environment, can facilitate the distribution of mist onto the growth matrix surface and/or extra-particle mycelial growth, or both. The air flow and misting apparatus can be tuned in concert to achieve the desired mist deposition rate and/or mean mist deposition rate, and to tune the mycelial tissue morphology.

Mist Deposition

“Mist deposition rate” as used herein refers to the rate at which mist is deposited per discrete instance of mist deposition. Any standalone usage herein of “mist deposition rate,” without the prefix “mean,” refers to the rate at which mist is deposited per discrete instance of mist deposition and is used interchangeably herein with “instantaneous mist deposition rate” or “momentary mist deposition rate.” “Mean mist deposition rate” is not used interchangeably herein with respect to “mist deposition rate” and is as defined elsewhere herein. The mist deposition rate can be based on or determined by measuring the volume of mist deposited on a surface area over a period of time, wherein the period of time is a fraction of the total incubation time period. In a non-limiting example, the mist is deposited on an exposed surface of growth matrix at a mist deposition rate of about 1 microliter per square centimeter of growth matrix per hour. In another non-limiting example, the mist is deposited on extra-particle aerial mycelial growth, and the mist deposition rate is about 1 microliter per square centimeter of the extra-particle aerial mycelial growth per hour. In some embodiments, the mist deposition rate can be reported as the volume of mist deposited per misting duty cycle. For the purposes of the present disclosure, a mist deposition rate of 1 microliter per centimeter squared per hour (1 uL/cm²/hour) is substantially equivalent to a mist deposition rate of 1 milligram per centimeter squared per hour (1 mg/cm²/hour), solute concentration notwithstanding.

“Mean mist deposition rate” as used herein refers to a mist deposition rate averaged over an incubation time period. The mean mist deposition rate can be expressed based on a surface area over which the mist is deposited. In a non-limiting example, the mist is deposited on an exposed surface of growth matrix at a mean mist deposition rate of about a microliter per square centimeter of growth matrix per hour. In another non-limiting example, the mist is deposited on an exposed surface of growth matrix containing extra-particle aerial mycelial growth, and the mean mist deposition rate is about 1 microliter per square centimeter of the growth matrix containing the extra-particle aerial mycelial growth per hour. For the purposes of the present disclosure, a mean mist deposition rate of 1 microliter per centimeter squared per hour (1 uL/cm²/hour) is substantially equivalent to a mean mist deposition rate of 1 milligram per centimeter squared per hour (1 mg/cm²/hour), solute concentration notwithstanding.

In some aspects, a method of growing aerial mycelium can include controlling homogenization of aerial growth by controlling mist deposition rate. The aerial growth response can be affected by the presence of mist in the growth environment, and/or by mist deposition in the growth environment, and/or by mist deposition on the growth matrix. Thus, in some embodiments, a method of making an aerial mycelium of the present disclosure can include exposing a growth matrix to a growth environment that has an amount of mist present therein. In some embodiments, exposing the growth matrix to the growth environment can include introducing aqueous mist into the growth environment. In some embodiments, aqueous mist can be introduced into the growth environment resulting in a detectable quantity of deposited mist in the growth environment. In some more particular embodiments, aqueous mist can be introduced into the growth environment resulting in a mean mist deposition rate that results in a detectable quantity of deposited mist in the growth environment. For example, aqueous mist can be introduced into the growth environment resulting in a mean mist deposition rate that results in a detectable quantity of deposited mist on surfaces of the container or other structure, on the growth matrix, on the extra-particle aerial mycelial growth, and/or on other structures within the growth environment. Methods of detecting deposited mist include visual inspection methods for visibly detectable deposited mist, measuring a quantity of deposited mist based on mass of collected mist or deposited solute, or other reasonable detection methods, as disclosed herein.

In some other embodiments, a growth environment can be provided that has an amount of mist present therein. The amount of mist present can be established before or during various actions taken within the growth environment, for example, during incubating a growth matrix. In some embodiments, the aqueous mist can be introduced into the growth environment resulting in a mean mist deposition rate that does not result in a detectable quantity of deposited mist in the growth environment. For example, the aqueous mist can be introduced into the growth environment resulting in a mean mist deposition rate that does not result in a detectable quantity of deposited mist on surfaces of the container or other structure, on the growth matrix, on the extra-particle aerial mycelial growth, and/or on other structures within the growth environment. Methods of detecting deposited mist include visual inspection methods for visibly detectable deposited mist, measuring a quantity of deposited mist based on mass of collected mist or deposited solute, or other reasonable detection methods.

The mean mist deposition rate within a growth environment is measurable by a variety of methods. A non-limiting example of a method of measuring an amount of deposited mist can be based upon the method of measuring mean mist deposition rate described below. Thus, in some embodiments, the aqueous mist can be introduced into the growth environment resulting in a mean mist deposition rate that does not result in a measurable mass of deposited mist in the growth environment. This can be confirmed after placing one or more open Petri dishes of known surface area in a growth environment during an incubation time period for at least 24 hours and at most about 7 days. Throughout this incubation time period, some amount of mist is present within the growth environment based upon some amount of mist introduction, allowing for the collection of some theoretical amount of deposited mist in the one or more open Petri dishes. The total theoretical mass of collected mist can be determined (to determine the mass of the deposited mist) and divided by the period of time (to determine the mean mist deposition rate based on mass). In embodiments wherein the aqueous mist present in the growth environment does not result in measurable mist deposition in the growth environment, the total amount (i.e., mass) of collected mist is negligible, i.e., not measurable within the tolerance of the balance used to determine the mass, but at some small amount above zero.

Mist deposition was assessed during abiotic (growth chamber absent fungal-inoculated growth media) and biotic trials (growth chamber containing fungal-inoculated growth media) using Celltreat 90×15 mm polypropylene Petri dishes, which were weighed without lids both before and after a mist deposition time period of 24 hours using an Intell-Lab Balance Model PM 300 having a resolution of 0.001 g, a minimum mass of 0.01 g, and a maximum mass of 300 g. Linearity between paired abiotic and biotic measurements at mean mist deposition rates in the range of 0 to 5 mg/cm²/hour showed a coefficient of variation (R²) of 0.98. A mass of collected mist of 0.01 g corresponded to a mean mist deposition rate of 0.006 mg/cm²/hour and gave rise to a film of moisture on the surface of the Petri dish that was visible to the naked eye. The limit of quantification (LOQ) of mean mist deposition rate was established as 0.006 mg/cm²/hour. The limit of detection (LOD) of the mean mist deposition rate was established as a rate below the LOQ that did not give rise to moisture on the Petri dish that was visible to the naked eye. For the purposes of the present disclosure, a mean mist deposition rate of 1 milligram per centimeter squared per hour (1 mg/cm²/hour) is substantially equivalent to 1 microliter per centimeter squared per hour (1 uL/cm²/hour), solute concentration notwithstanding. Accordingly, the LOQ of mean mist deposition rate can be expressed as 0.006 uL/cm²/hour, and the LOD of the mean mist deposition rate can be expressed as a rate below 0.006 uL/cm²/hour that did not give rise to moisture on the Petri dish that was visible to the naked eye.

It is hypothesized that the aforementioned methods for assessing the mean mist deposition rate with respect to a Petri dish in a growth environment would correlate and thus provide similar results for assessing these same variables on the container, on the growth matrix, on the extra-particle aerial mycelial growth, and/or on other structures within the growth environment. Additionally, the aforementioned methods can be applied and thus would correlate and provide similar results for assessing the mist deposition rate (i.e., instantaneous mist deposition rate) by dividing the mean mist deposition rate by the duty cycle.

In some other embodiments, aqueous mist can be introduced into the growth environment resulting in a mean mist deposition rate that does not result in a measurable volume of deposited mist in the growth environment. This can be confirmed after placing one or more open Petri dishes of known surface area in a growth environment during an incubation time period for at least 24 hours and at most about 7 days. Throughout this incubation time period, some amount of mist is present within the growth environment based upon some amount of mist introduction, allowing for the collection of some theoretical volume of deposited mist in the one or more open Petri dishes. The total theoretical volume of collected mist can be determined (to determine the volume of the deposited mist) and divided by the period of time (to determine the mean mist deposition rate based on volume). In embodiments wherein the aqueous mist present in the growth environment does not result in measurable mist deposition in the growth environment based on volume, the total amount (i.e., volume) of collected mist is negligible, i.e., not measurable within the tolerance of the volumetric equipment used to determine the volume, but at some small amount above zero.

In yet other embodiments, the aqueous mist can be introduced into the growth environment resulting in a mean mist deposition rate that does not result in visible deposited mist in the growth environment. This can be confirmed after placing one or more open Petri dishes in a growth environment during an incubation time period for at least 24 hours and at most about 7 days. Throughout this incubation time period, some amount of mist is present within the growth environment based upon some amount of mist introduction, allowing for the collection of some theoretical amount of deposited mist in the one or more open Petri dishes. During and/or upon completion of the incubation time period, the one or more open Petri dishes can be visually inspected to confirm that no visible amount of mist deposition is present (functionally, when the one or more Petri dishes are dry).

In some aspects, the total volume of aqueous mist introduced into the growth environment throughout the incubation period, or a portion thereof, is less than about 200 uL/cm², is less than about 100 uL/cm², is less than about 50 uL/cm², is less than about 25 uL/cm², is less than about 20 uL/cm², is less than about 15 uL/cm², or is less than about 10 uL/cm². In some further aspects, the total volume of aqueous mist introduced into the growth environment throughout the incubation period, or a portion thereof, is at least about 5 uL/cm².

In some aspect of the present disclosure, aqueous mist can contain one or more dissolved solutes. US 2020/0146224, the entire contents of which are hereby incorporated by reference in their entirety to the extent not inconsistent with the content of this disclosure, discloses a method of growing a mycelium biopolymer material comprising placing the plurality of containers in a closed incubation chamber and distributing a mist throughout the incubation chamber for passage over the growth media in each container, wherein the mist includes moisture and a solute, such as minerals. US 2020/0146224 further discloses that growing tissue in each container comprises aerial hypha growing up and out of a nutritious space into a non-nutrient environment, and that, in order to control growth in such an environment, the organism employs the use of turgor pressure to regulate the extension of the hyphae at the hyphal tip; thus, regulating the amount, distribution and/or droplet size of available moisture and solutes deposited across the top surface of the growing material can control the osmotic gradient created within the hyphae and subsequently, its growth rate and pattern of colonization. Surprisingly, Applicant has discovered that aerial mycelial growth can be produced by introducing aqueous mist into the growth environment, which can result in depositing aqueous mist in the growth environment, wherein the aqueous mist deposits substantially no amount of dissolved solute onto the growth matrix and/or the extra-particle aerial mycelial growth produced therefrom. In some aspects, a method of making an aerial mycelium can include introducing aqueous mist sourced from tap water having a conductivity within a range of 400 to 500 microsiemens/cm. In other aspects, a method of making aerial mycelium can include introducing aqueous mist sourced from reverse osmosis filtered water having a conductivity within a range of 20 to 40 microsiemens/cm. In yet other aspects, a method of making aerial mycelium can include introducing aqueous mist sourced from distilled water having a conductivity of about 3 microsiemens/cm. In addition to Applicant's discovery of a binary aerial growth response to the binary application of aqueous mist (i.e., aerial mycelial growth in response to presence of aqueous mist vs. reduced or no growth in absence of aqueous mist), Applicant has further discovered that this binary response is observed even when the aqueous mist contains substantially no amounts of dissolved solute. Moreover, the aerial mycelia of the present disclosure have properties including their native thickness that exceed those observed under standard culture conditions and exceed those of any mycelia found in nature.

Thus, in some aspects, the present disclosure provides for a method of growing an aerial mycelium in a growth environment, the growth environment comprising a growth matrix and aqueous mist (or the method comprising introducing the aqueous mist), wherein the aqueous mist can have a conductivity of no greater than about 500 microsiemens/cm. In some further aspects, the aqueous mist conductivity can be no greater than about 400 microsiemens/cm, no greater than about 300 microsiemens/cm, no greater than about 200 microsiemens/cm, or no greater than about 100 microsiemens/cm. In some other aspects, the aqueous mist conductivity can be no greater than about 50 microsiemens/cm, no greater than about 40 microsiemens/cm, no greater than about 30 microsiemens/cm, no greater than about 20 microsiemens/cm, no greater than about 10 microsiemens/cm, or no greater than about 5 microsiemens/cm.

As disclosed herein, in some embodiments, the mist comprises one or more solutes. In some embodiments, the one or more solutes is an additive. Non-limiting examples of additives include mineral nutrients, additives for pH adjustment, additives for electrical conductivity adjustment, soluble sugars, nitrogen sources, or other water-soluble additives. Non-limiting examples of mineral nutrients include calcium chloride, potassium phosphate, magnesium sulfate, ferrous sulfate, manganese sulfate, and copper sulfate. Non-limiting examples of additives for pH adjustment include hydrochloric acid and sodium hydroxide. Non-limiting examples of sugars include glucose, xylose, maltose, maltotriose, and cellobiose. Non-limiting examples of nitrogen sources include peptone, urea, and glutamic acid.

In some further aspects of the disclosure, the mist that is introduced into the growth environment is characterized as having a mist deposition rate and a mean mist deposition rate.

In some embodiments, the mist deposition rate is less than about 50 uL/cm²/hour, is less than about 25 uL/cm²/hour, is less than about 15 uL/cm²/hour, is less than about 10 uL/cm²/hour, is less than about 5 uL/cm²/hour, is less than about 4 uL/cm²/hour, is less than about 3 uL/cm²/hour or is less than about 2 uL/cm²/hour. In some more particular embodiments, the mist deposition rate is less than about 1 uL/cm²/hour. In some further embodiments, the mist deposition rate is at least about 0.01 uL/cm²/hour, is at least about 0.02 uL/cm²/hour, is at least about 0.03 uL/cm²/hour, is at least about 0.04 uL/cm²/hour, or is at least about 0.05 uL/cm²/hour. In yet some further embodiments, the mist deposition rate is within a range of: about 0.05 to about 0.8 uL/cm²/hour, about 0.05 to about 0.75 uL/cm²/hour, about 0.1 to about 0.8 uL/cm²/hour, about 0.1 to about 0.75 uL/cm²/hour, about 0.2 to about 0.8 uL/cm²/hour, about 0.2 to about 0.75 uL/cm²/hour, about 0.2 to about 0.7 uL/cm²/hour, about 0.2 to about 0.6 uL/cm²/hour, about 0.2 to about 0.5 uL/cm²/hour, about 0.2 to about 0.4 uL/cm²/hour, about 0.3 to about 0.5 uL/cm²/hour, about 0.3 to about 0.4 uL/cm²/hour or about 0.30 to about 0.35 uL/cm²/hour. In yet more particular embodiments still, the mist deposition rate is about 0.01 uL/cm²/hour, about 0.02 uL/cm²/hour, about 0.03 uL/cm²/hour, about 0.04 uL/cm²/hour, about 0.05 uL/cm²/hour, about 0.10 uL/cm²/hour, about 0.15 uL/cm²/hour, about 0.20 uL/cm²/hour, about 0.25 uL/cm²/hour, about 0.30 uL/cm²/hour, about 0.35 uL/cm²/hour, about 0.40 uL/cm²/hour, about 0.45 uL/cm²/hour, about 0.50 uL/cm²/hour, about 0.55 uL/cm²/hour, about 0.60 uL/cm²/hour, about 0.65 uL/cm²/hour, about 0.70 uL/cm²/hour, about 0.75 uL/cm²/hour, about 0.80 uL/cm²/hour, about 0.85 uL/cm²/hour, about 0.90 uL/cm²/hour, or about 0.95 uL/cm²/hour, or any range therebetween.

In some embodiments, the mist deposition rate is less than about 1 μL/cm²/hour, is less than about 0.9 μL/cm²/hour, is less than about 0.8 μL/cm²/hour, is less than about 0.7 μL/cm²/hour, is less than about 0.6 μL/cm²/hour, is less than about 0.5 μL/cm²/hour, is less than about 0.4 μL/cm²/hour, is less than about 0.3 μL/cm²/hour or is less than about 0.2 μL/cm²/hour. In some more particular embodiments, the mist deposition rate is less than about 1.25 μL/cm²/hour, or in some embodiments, less than about 1.0 μL/cm²/hour. In some further embodiments, the mist deposition rate is at least about 0.2 μL/cm²/hour, is at least about 0.3 μL/cm²/hour, is at least about 0.4 μL/cm²/hour, is at least about 0.5 μL/cm²/hour, or is at least about 0.6 μL/cm²/hour. In yet some further embodiments, the mist deposition rate is within a range of: about 0.2 to about 0.3 μL/cm²/hour, about 0.2 to about 0.4 μL/cm²/hour, about 0.2 to about 0.5 μL/cm²/hour, about 0.2 to about 0.6 μL/cm²/hour, about 0.2 to about 0.7 μL/cm²/hour, about 0.2 to about 0.8 μL/cm²/hour, about 0.2 to about 0.9 μL/cm²/hour, about 0.2 to about 1 μL/cm²/hour, about 0.2 to about 1.25 μL/cm²/hour, or any range therebetween.

In some embodiments, the mean mist deposition rate is less than or equal to about 10 uL/cm²/hour, is less than or equal to about 5 uL/cm²/hour, is less than or equal to about 4 uL/cm²/hour, is less than or equal to about 3 uL/cm²/hour or is less than or equal to about 2 uL/cm²/hour. In some embodiments, the mean mist deposition rate is less than or equal to about 1 uL/cm²/hour, is less than or equal to about 0.95 uL/cm²/hour, is less than or equal to about 0.9 uL/cm²/hour, is less than or equal to about 0.85 uL/cm²/hour, is less than or equal to about 0.8 uL/cm²/hour, is less than or equal to about 0.75 uL/cm²/hour, is less than or equal to about 0.7 uL/cm²/hour, is less than or equal to about 0.65 uL/cm²/hour, is less than or equal to about 0.6 uL/cm²/hour, is less than or equal to about 0.55 uL/cm²/hour, or is less than or equal to about 0.5 uL/cm²/hour. In some further embodiments, the mean mist deposition rate is at least about 0.01 uL/cm²/hour, is at least about 0.02 uL/cm²/hour, is at least about 0.03 uL/cm²/hour, is at least about 0.04 uL/cm²/hour or is at least about 0.05 uL/cm²/hour. In yet some further embodiments, the mean mist deposition rate is within a range of: about 0.01 to about 10 uL/cm²/hour, about 0.01 to about 5 uL/cm²/hour, about 0.01 to about 4 uL/cm²/hour, about 0.01 to about 3 uL/cm²/hour, about 0.01 to about 2 uL/cm²/hour, about 0.01 to about 1 uL/cm²/hour, about 0.01 to about 0.9 uL/cm²/hour, about 0.01 to about 0.8 uL/cm²/hour, about 0.01 to about 0.75 uL/cm²/hour, about 0.01 to about 0.7 uL/cm²/hour, about 0.02 to about 10 uL/cm²/hour, about 0.02 to about 5 uL/cm²/hour, about 0.02 to about 4 uL/cm²/hour, about 0.02 to about 3 uL/cm²/hour, about 0.02 to about 2 uL/cm²/hour, about 0.02 to about 1 uL/cm²/hour, about 0.02 to about 0.9 uL/cm²/hour, about 0.02 to about 0.8 uL/cm²/hour, about 0.02 to about 0.75 uL/cm²/hour, about 0.02 to about 0.7 uL/cm²/hour, about 0.03 to about 10 uL/cm²/hour, about 0.03 to about 5 uL/cm²/hour, about 0.03 to about 4 uL/cm²/hour, about 0.03 to about 3 uL/cm²/hour, about 0.03 to about 2 uL/cm²/hour, about 0.03 to about 1 uL/cm²/hour, about 0.03 to about 0.9 uL/cm²/hour, about 0.03 to about 0.8 uL/cm²/hour, about 0.03 to about 0.75 uL/cm²/hour, about 0.03 to about 0.7 uL/cm²/hour, about 0.04 to about 10 uL/cm²/hour, about 0.04 to about 5 uL/cm²/hour, about 0.04 to about 4 uL/cm²/hour, about 0.04 to about 3 uL/cm²/hour, about 0.04 to about 2 uL/cm²/hour, about 0.04 to about 1 uL/cm²/hour, about 0.04 to about 0.9 uL/cm²/hour, about 0.04 to about 0.8 uL/cm²/hour, about 0.04 to about 0.75 uL/cm²/hour, about 0.04 to about 0.7 uL/cm²/hour, about 0.05 to about 10 uL/cm²/hour, about 0.05 to about 5 uL/cm²/hour, about 0.05 to about 4 uL/cm²/hour, about 0.05 to about 3 uL/cm²/hour, about 0.05 to about 2 uL/cm²/hour, about 0.05 to about 1 uL/cm²/hour, about 0.05 to about 0.9 uL/cm²/hour, about 0.05 to about 0.8 uL/cm²/hour, about 0.05 to about 0.75 uL/cm²/hour, about 0.05 to about 0.7 uL/cm²/hour, about 0.1 to about 10 uL/cm²/hour, about 0.1 to about 5 uL/cm²/hour, about 0.1 to about 4 uL/cm²/hour, about 0.1 to about 3 uL/cm²/hour, about 0.1 to about 2 uL/cm²/hour, about 0.1 to about 1 uL/cm²/hour, about 0.1 to about 0.9 uL/cm²/hour, about 0.1 to about 0.8 uL/cm²/hour, about 0.1 to about 0.75 uL/cm²/hour, about 0.1 to about 0.7 uL/cm²/hour, about 0.2 to about 10 uL/cm²/hour, about 0.2 to about 5 uL/cm²/hour, about 0.2 to about 4 uL/cm²/hour, about 0.2 to about 3 uL/cm²/hour, about 0.2 to about 2 uL/cm²/hour, about 0.2 to about 1 uL/cm²/hour, about 0.2 to about 0.9 uL/cm²/hour, about 0.2 to about 0.8 uL/cm²/hour, about 0.2 to about 0.75 uL/cm²/hour, about 0.2 to about 0.7 uL/cm²/hour, about 0.2 to about 0.6 uL/cm²/hour, about 0.2 to about 0.5 uL/cm²/hour, about 0.2 to about 0.4 uL/cm²/hour, about 0.3 to about 0.5 uL/cm²/hour, about 0.3 to about 0.4 uL/cm²/hour or about 0.30 to about 0.35 uL/cm²/hour. In some more particular embodiments, the mean mist deposition rate is about 0.05 microliters/cm²/hour, about 0.10 microliters/cm²/hour, about 0.15 microliters/cm²/hour, about 0.20 microliters/cm²/hour, about 0.25 microliters/cm²/hour, about 0.30 microliters/cm²/hour, about 0.35 microliters/cm²/hour, about 0.40 uL/cm²/hour, about 0.45 uL/cm²/hour, about 0.50 uL/cm²/hour, about 0.55 uL/cm²/hour, about 0.60 uL/cm²/hour, about 0.65 uL/cm²/hour, about 0.70 uL/cm²/hour, about 0.75 uL/cm²/hour, about 0.80 uL/cm²/hour, about 0.85 uL/cm²/hour, about 0.90 uL/cm²/hour, about 0.95 uL/cm²/hour, or about 1.0 uL/cm²/hour, or any range therebetween.

In some embodiments, the mist deposition rate is at most about 20-fold greater than the mean mist deposition rate. In some embodiments, the mist deposition rate is at most about 10-fold greater than the mean mist deposition rate. In some further embodiments, the mist deposition rate is at most about 5-fold greater, is at most 4-fold greater, is at most about 3-fold greater, or is at most about 2-fold greater than the mean mist deposition rate. In some embodiments, the mist deposition rate is substantially the same as the mean mist deposition rate. In some more particular embodiments, the mist deposition rate is less than about 2 uL/cm²/hour and the mean mist deposition rate is less than about 1 uL/cm²/hour. In yet further embodiments, the mist deposition rate and the mean mist deposition rate are each less than about 1 uL/cm²/hour. In yet further embodiments still, the mist deposition rate is less than about 1 uL/cm²/hour, and the mean mist deposition rate is less than about 0.5 uL/cm²/hour.

In other embodiments, the mist deposition rate is at most about 150 uL/cm²/hour, is at most about 100 uL/cm²/hour, is at most about 75 uL/cm²/hour, is at most about 50 uL/cm²/hour or is at most about 25 uL/cm²/hour. In some further embodiments, the mist deposition rate is at least about 10 uL/cm²/hour or is at least about 15 uL/cm²/hour. In some embodiments, the mist deposition rate is at most about 100 uL/cm²/hour, and the mean mist deposition rate is at least about 10 uL/cm²/hour or is at least about 15 uL/cm²/hour.

Applicant has discovered that aerial mycelial growth can be produced from a growth matrix in a growth environment comprising very low levels of aqueous mist. Reducing moisture content of an aerial mycelium can advantageously reduce the risk of microbial contamination. Thus, in some aspects of the present disclosure, mist deposition rates and mean mist deposition rates can be tuned to drive a target native moisture content of an aerial mycelium. In some aspects of the present disclosure, there is provided a method of making an aerial mycelium comprising incubating a growth matrix in a growth environment for an incubation time period, wherein the growth matrix comprises a substrate and a fungus; introducing aqueous mist into the growth environment throughout the incubation time period or a portion thereof; and producing extra-particle aerial mycelial growth from the growth matrix; wherein introducing comprises introducing aqueous mist into the growth environment resulting in a mean mist deposition rate of at most about 0.45 uL/cm²/hour. In some embodiments, introducing comprises introducing aqueous mist into the growth environment resulting in an instantaneous mist deposition rate and a mean mist deposition rate, wherein the ratio of the instantaneous mist deposition rate to the mean mist deposition rate is at most about 20 to about 1. In some embodiments, the ratio of the instantaneous mist deposition rate to the mean mist deposition rate is at most about 10 to about 1. In some embodiments, the ratio of the instantaneous mist deposition rate to the mean mist deposition rate is at most about 5 to about 1. In some embodiments, introducing further comprises introducing aqueous mist into the growth environment resulting in an instantaneous mist deposition rate of at most about 2 uL/cm²/hour. In some embodiments, the introducing comprises introducing aqueous mist into the growth environment resulting in a mean mist deposition rate of at most about 0.40 uL/cm²/hour, at most about 0.35 uL/cm²/hour, at most about 0.30 uL/cm²/hour, at most about 0.25 uL/cm²/hour, at most about 0.20 uL/cm²/hour, at most about 0.15 uL/cm²/hour, or at most about 0.10 uL/cm²/hour. In some embodiments, the mean mist deposition rate is at least about 0.01 uL/cm²/hour. In some embodiments, the growth matrix comprises a substrate and a fungal inoculum, said fungal inoculum comprising a fungus. In some other embodiments, the growth matrix comprises a colonized substrate, said colonized substrate comprising a substrate, wherein said substrate is previously colonized with mycelium of a fungus. In some embodiments, the colonized substrate is a fragmented colonized substrate.

In some other aspects of the present disclosure, there is provided a method of making an aerial mycelium comprising introducing aqueous mist into a growth environment; incubating a growth matrix in a growth environment for an incubation time period, wherein the growth matrix comprises a substrate and a fungus, and wherein incubating comprises exposing the growth matrix to aqueous mist; and producing extra-particle aerial mycelial growth from the growth matrix; wherein introducing comprises introducing aqueous mist into the growth environment resulting in a mean mist deposition rate that is below about 0.01 uL/cm²/hour. In some embodiments, introducing comprises introducing aqueous mist into the growth environment resulting in a mean mist deposition rate that does not result in a detectable quantity of deposited mist in the growth environment. In some embodiments, the growth matrix comprises the substrate and a fungal inoculum, said fungal inoculum comprising a fungus. In some other embodiments, the growth matrix comprises a colonized substrate, said colonized substrate comprising a substrate, wherein said substrate is previously colonized with mycelium of a fungus. In some embodiments, the colonized substrate is a fragmented colonized substrate.

In some non-limiting embodiments, aqueous mist can be introduced into the growth environment via a misting apparatus, which can be incorporated into the growth environment. The apparatus that introduces the aqueous mist can be the same as or different from an apparatus that controls relative humidity of the growth environment. Non-limiting examples of a misting apparatus suitable for introducing mist into the growth environment include a high pressure misting pump, a nebulizer, an aerosol generator or aerosolizer, a mist generator, an ultrasonic nebulizer, an ultrasonic aerosol generator or aerosolizer, an ultrasonic mist generator, a dry fog humidifier, an ultrasonic humidifier, or an atomizer misting system (including but not limited to a “misting puck”), essentially as described in WO 2019/099474 A1, the entire contents of which is hereby incorporated by reference in its entirety to the extent not inconsistent with the content of this disclosure, or a print head configured to deposit mist, such as a 3D printer, essentially as described in U.S. patent application publication no. 2020/0157506A1, the entire contents of which is hereby incorporated by reference in its entirety to the extent not inconsistent with the content of this disclosure. In some other non-limiting embodiments, mist can be introduced into the growth environment via modulation of growth environmental factors such as growth environment atmospheric pressure, temperature and/or relative humidity, or via modulation of the growth atmosphere dew point.

In some embodiments, mist can be continuously introduced into the growth environment. In some further embodiments, the continuous introduction of mist can be pulse width modulated. In some other embodiments, the continuous introduction of mist deposition can occur at a fixed rate. In yet some other embodiments, the continuous introduction of mist deposition can occur at a variable rate.

In other embodiments, mist can be intermittently introduced into the growth environment. In some further embodiments, the intermittent introduction of mist can occur at a fixed rate. In other further embodiments, the intermittent introduction of mist can occur at a variable rate. In other further embodiments, the intermittent introduction of mist can occur at regular or irregular periods. In other further embodiments, the intermittent introduction of mist can occur with regular or irregular intervals therebetween without mist introduction.

“Duty Cycle” as used herein is the percentage of the given mist cycle period over which mist is provided. In some embodiments, a misting apparatus can be operated at a particular duty cycle. In some embodiments, the misting apparatus is operated at a duty cycle of about 100%. In some embodiments, the misting apparatus is operated at a duty cycle within a range of about 0.1% to about 100%. In some embodiments, the misting apparatus is operated at a duty cycle within a range of about 1% to about 100%, about 5% to about 100%, about 10% to about 100%, about 15% to about 100%, about 20% to about 100% or about 25% to about 100%. In some other embodiments, the misting apparatus is operated at a duty cycle of less than 100%. In some embodiments, the misting apparatus is operated at a duty cycle of no greater than about 75%, no greater than about 50%, no greater than about 40%, no greater than about 30%, no greater than about 25%, no greater than about 20% or no greater than about 15%. In some further embodiments, the misting apparatus is operated at a duty cycle of at least about 1%, at least about 5%, at least about 10%, at least about 15%, at least about 20% or at least about 25%. In some more particular embodiments, the misting apparatus is operated within a range of about 1% to about 15%, about 5% to about 25%, about 25% to about 50%, about 50% to about 75%, or about 75% to about 100%.

In some embodiments, a duty cycle can be further characterized by a “mist cycle period.” Non-limiting examples include a duty cycle period of about 3600 second (i.e., about 1 hour), about 1800 seconds (i.e., about 30 minutes), about 360 seconds, (i.e., about 6 minutes), about 180 seconds (i.e., about 3 minutes), or about 60 seconds (i.e., about 1 minute), or any value or range therebetween. In some embodiments, a duty cycle period can be at most about 60 minutes, at most about 30 minutes, at most about 15 minutes, or at most about 10 minutes. In some other embodiments, a duty cycle period can be at most about 9 minutes, at most about 8 minutes, at most about 7 minutes or at most about 6 minutes.

As disclosed herein, a method of making an aerial mycelium of the present disclosure can include introducing aqueous mist into the growth environment throughout an incubation time period. Introducing aqueous mist “throughout the incubation time period” as used herein refers to introducing the aqueous mist from the beginning of the incubation time period to the end of the incubation time period. In some aspects, introducing aqueous mist into the growth environment can comprise operating a misting apparatus at a duty cycle of greater than zero from the beginning of the incubation time period to the end of the incubation time period. In a non-limiting example, introducing aqueous mist into a growth environment throughout the incubation time period can comprise operating a misting apparatus at a 50% duty cycle from the beginning of the incubation time period to the end of the incubation time period. Further to this non-limiting example, the misting apparatus operating at the 50% duty cycle can have a duty cycle period of at most about 10 minutes. Thus, in this non-limiting example, the misting apparatus can operate (and thus release mist) for 5 minutes out of each 10-minute duty cycle period, and each 10-minute duty cycle period repeats from the beginning of the incubation time period to the end of the incubation time period. Similarly, introducing mist “throughout a portion of the incubation time period” as used herein refers to introducing the mist from the beginning of the portion of the incubation time period to the end of the portion of the incubation time period. In some embodiments, the end of the portion of the incubation time period can be the end of the entire incubation time period. In some aspects, introducing aqueous mist into the growth environment throughout a portion of the incubation time period can comprise operating a misting apparatus at a duty cycle of greater than zero from the beginning of the portion of the incubation time period to the end of the portion of the incubation time period. It will be understood that introducing aqueous mist “throughout the incubation time period” and “throughout a portion of the incubation time period” as used herein can include, but does not require, mist introduction at exactly the beginning or at exactly the end of the incubation time period or during a portion of the incubation time period, for example, in embodiments where the mist is not applied continuously throughout the entirety of the incubation time period or a portion of the incubation time period.

In some aspects, the present disclosure provides for an aqueous mist characterized as having a mean droplet diameter. In some embodiments, the aqueous mist has a droplet diameter within a range of about 1 to about 30 microns (um), within a range of about 1 to about 25 microns, within a range of about 1 to about 20 microns, within a range of about 1 to about 15 microns, within a range of about 1 to about 10 microns, or within a range of about 5 to about 10 microns.

The present disclosure provides for a growth environment atmosphere that is characterized as having a relative humidity sufficient to support mycelial growth. In some aspects, a growth environment atmosphere of the present disclosure can have a relative humidity of greater than 99.5%. In some other embodiments, a growth environment atmosphere of the present disclosure can have a relative humidity of at least about 97%. In some even more particular embodiments, the growth environment atmosphere can have a relative humidity of at least about 98%. In yet more particular embodiments still, the growth environment atmosphere can have a relative humidity of at least about 99% or can have a relative humidity of about 100%. In some embodiments, the growth environment atmosphere can have a relative humidity of at least about 99.0%, 99.1%, 99.2%, 99.3%, 99.4%, 99.5%, 99.6%, 99.7%, 99.8% or 99.9%; or any range therebetween. In some embodiments, the growth environment atmosphere can have a relative humidity of about 100%. In some further embodiments still, the growth environment atmosphere can have a relative humidity of greater than 100%. In some embodiments, the growth environment atmosphere can be a saturated atmosphere. In some other embodiments, the growth environment atmosphere can be a supersaturated atmosphere. As used herein, a “supersaturated atmosphere” refers to an atmosphere wherein the relative humidity is greater than 100%. Regardless of the relative humidity of the growth environment atmosphere, a growth environment of the present disclosure that is suitable for producing aerial mycelium contains liquid phase water in the form of aqueous mist. Thus, even in a growth environment having a saturated or supersaturated atmosphere, methods of growing aerial mycelia of the present disclosure can include introducing aqueous mist to the growth environment; accordingly, a growth environment of the present disclosure can include a saturated or supersaturated growth atmosphere plus aqueous mist that is introduced from a source other than the water vapor held in the saturated or supersaturated atmosphere. In sum, a growth environment of the present disclosure contains water vapor and droplets of liquid water in the form of aqueous mist.

Means of introducing and regulating relative humidity of a growth environment suitable for the growth of mycelia would be readily understood by a person of ordinary skill in the art in the mycelial cultivation industry. In some embodiments, the relative humidity can be controlled independent of misting using conventional heating, ventilation, and air conditioning (HVAC) practices. For example, gaseous moisture can be added to the growth environment by introducing steam into the growth atmosphere via such conventional HVAC practices. In other embodiments, an interplay between the gas phase water vapor and liquid phase aqueous mist can be exploited. Accordingly, aqueous mist can be introduced into the growth environment at an increased or decreased rate as a means of modifying the growth environment relative humidity.

Time

Aerial mycelia of the present disclosure can be grown in a matter of weeks or days. This feature is of especially practical value in the production of food ingredients or food products, where time and efficiency are at a premium. Accordingly, the presently disclosed method of making an aerial mycelium comprises incubating a growth matrix in a growth environment for an incubation time period of up to about 3 weeks. In some embodiments, the incubation time period can be within a range of about 4 days to about 17 days. In some further embodiments, the incubation time period can be within a range of about 7 days to about 16 days, within a range of about 8 days to about 15 days, within a range of about 9 days to about 15 days, within a range of about 9 days to about 14 days, within a range of about 8 to about 14 days, within a range of about 7 to about 13 days, or within a range of about 7 to about 10 days. In some more particular embodiments, the incubation time period can be about 7 days, about 8 days, about 9 days, about 10 days, about 11 days, about 12 days, about 13 days, about 14 days, about 15 days or about 16 days, or any range therebetween.

Advantageously, incubating a growth matrix comprising a colonized substrate (wherein said colonized substrate comprises a growth medium previously colonized with mycelium of a fungus) in a growth environment of the present disclosure can result in earlier expression of aerial mycelial tissue compared to incubation of a growth matrix comprising substantially the same or a similar growth medium and a fungal inoculum, wherein the fungal inoculum contains a fungus. Accordingly, a method of making an aerial mycelium of the present disclosure can comprise incubating a growth matrix comprising a colonized substrate (wherein said colonized substrate comprises a growth medium previously colonized with mycelium of a fungus) in a growth environment for an incubation time period, and producing extra-particle aerial mycelial growth therefrom, wherein the incubation time period is at least about 1 day, at least about 2 days, at least about 3 days, or at least about 4 days less than the incubation time period for producing extra-particle aerial mycelial growth from a growth matrix comprising a growth medium and a fungal inoculum, wherein the fungal inoculum comprises a fungus.

In some other embodiments, the incubation time period ends no later than when a visible fruiting body forms. In a non-limiting example, the incubation time period can end prior to a karyogamy or meiosis phase of the fungal reproductive cycle. In some other embodiments, the incubation time period ends when a visible fruiting body forms. As disclosed herein, aerial mycelia of the present disclosure can be prepared without the formation of a visible fruiting body, thus, in some embodiments, an incubation time period can end without regard to the formation of a visible fruiting body. Trial incubation runs can be used to inform the period of time in the growth environment during which sufficient extra-particle aerial mycelial growth product occurs (e.g., aerial mycelial growth of a predetermined thickness) without the formation of visible fruiting bodies.

In some embodiments, the method of making an aerial mycelium of the present disclosure can comprise introducing aqueous mist into the growth environment throughout the incubation time period.

Beyond the discovery of a binary aerial growth response to aqueous mist (supra), Applicant has also discovered that aerial mycelia of the present disclosure can be prepared by exposing a growth matrix to aqueous mist throughout a portion of the incubation time period (e.g., by introducing mist into the growth environment throughout a portion of the incubation time period). Applicant has measured vertical expansion kinetics of mycelia over the course of an entire incubation period and has characterized the kinetics as having a primary myceliation phase and a vertical expansion phase. The primary myceliation phase included days 1 to 3 of the incubation time period. Introducing aqueous mist throughout a portion of the incubation time period (wherein the portion included the vertical expansion phase), and not introducing aqueous mist on days 1 to 3 of the incubation time period was sufficient to produce aerial mycelium having substantially similar characteristics to aerial mycelia obtained by depositing mist throughout the entire incubation period.

Thus, while some aspects of the present disclosure provide for a method of making an aerial mycelium comprising exposing a growth matrix to a growth environment comprising aqueous mist throughout the incubation time period (e.g., by introducing aqueous mist into the growth environment throughout the incubation time period, i.e., throughout the entire incubation time period), in other aspects, the present disclosure provides for a method of making an aerial mycelium comprising exposing a growth matrix to aqueous mist throughout a portion of the incubation time period (e.g., by introducing aqueous mist into the growth environment throughout a portion of the incubation time period). In some embodiments, a portion of the incubation time period can comprise a vertical expansion phase. In some further embodiments, a portion of the incubation time period can further comprise at least a portion of a primary myceliation phase. In some other embodiments, a portion of the incubation time period can exclude a primary myceliation phase. In yet some other embodiments, a portion of the incubation time period can comprise a vertical expansion phase. Accordingly, in some aspects, introducing aqueous mist into a growth environment throughout a portion of an incubation time period can comprise introducing aqueous mist into the growth environment throughout a vertical expansion phase. In some embodiments, introducing aqueous mist into the growth environment throughout a portion of the incubation time period can comprise introducing aqueous mist into the growth environment throughout a vertical expansion phase and can exclude introducing aqueous mist during the primary myceliation phase. In some embodiments, the portion of the incubation time period can terminate at the end of a vertical expansion phase or can terminate at the end of an incubation time period.

In some other aspects, a portion of an incubation time period can begin during a first day, a second day, a third day or a fourth day of the incubation time period. Accordingly, in some aspects, introducing aqueous mist into a growth environment throughout a portion of an incubation time period can comprise introducing aqueous mist into the growth environment during a first, a second, a third or a fourth day of the incubation time period. In some embodiments, the portion of the incubation time period can terminate at the end of a vertical expansion phase or can terminate at the end of an incubation time period.

### Methods Relating to Topology Adjustment Layers

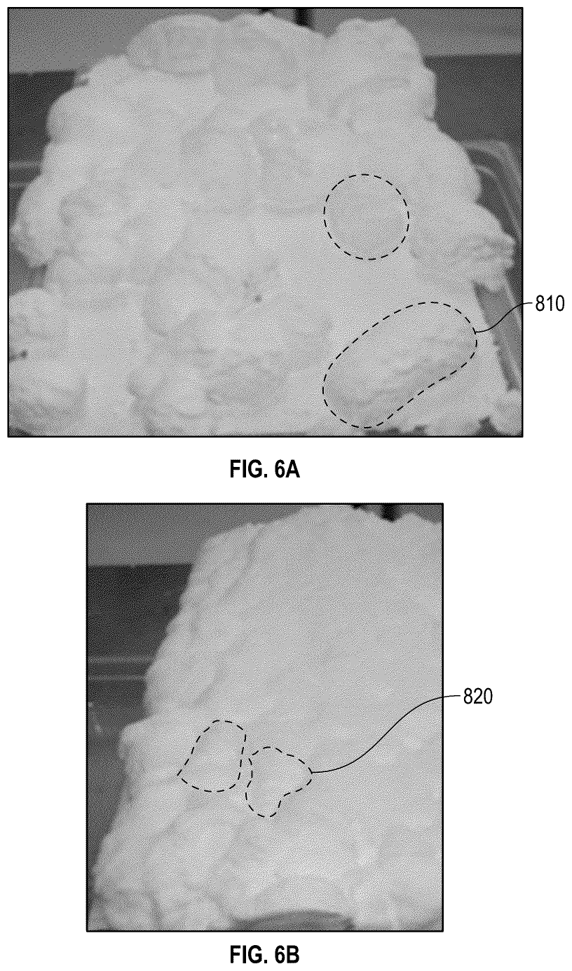

FIG. 6 illustrates aerial mycelium morphologies on an aerial mycelium panel grown from the control cultivation paradigm, in accordance with embodiments for growing aerial mycelium as disclosed in U.S. Pat. No. 11,266,085, issued Mar. 8, 2022, the entire contents of which is hereby incorporated by reference in its entirety to the extent not inconsistent with the content of this disclosure.

The topological morphology of the aerial mycelium panel is generally composed of bulbous forms which may be discrete 810, forming a dis-contiguous panel (FIG. 6A), or bulbous forms which may be diffusive and fused together 820, forming a contiguous panel (FIG. 6B). Of the bulbous forms 810 and 820, the pattern of expression, size and shape of these bulbous forms are generally variable and irregular. Density of the tissue generally presents radially within a given bulbous structure (either discrete 810 or fused 820), where the densest tissue is at the core and density decreases toward the margins of the bulbous form, resulting in the lowest density in the overlapping or fused regions between bulbous forms.

A method for affecting the growth topology of aerial mycelium, including, for example, growing aerial mycelium with increased homogeneity, is described herein. The increased homogeneity can include a reduced number of discrete bulbous features, a lack of discrete bulbous features, or a plurality of discrete bulbous features wherein such bulbous features have so intersected and merged that the overall topography of the overall material includes few if any visually discernible low or high points in the vertical (Z) direction as observed by an observer without visual impairment from a distance of about 12 inches.

A method for growing aerial mycelium with increased geometric regularity is also described herein. The geometric regularity can include expression of bulbous features in a defined geometry such that textural, aesthetic, and mechanical qualities are reproducible.

In various aspects, the method for affecting the growth topology of aerial mycelium can include applying spawn to a substrate surface in a defined pattern. The method can further include depositing spawn by hand, screen printing, slurry inkjet printing, or removing spawn in a defined pattern. The depositing can be done according to a defined pattern that can vary in the exact pattern and spacing across a surface.

In various aspects, the method for affecting the growth topology of aerial mycelium can include applying an uninoculated substrate. The uninoculated substrate can vary in composition and thickness. In some aspects, the method for growing aerial mycelium can include applying an uninoculated substrate layer above and/or below the applied pattern of spawn.

In some aspects, the method for affecting the growth topology of aerial mycelium can include applying an inoculated substrate. In various aspects, inoculating can be done via printing, by applying liquid inoculum, or by applying solid inoculum. In other aspects, an entire substrate can be inoculated, and a treatment can be further applied in a manner to kill some inoculum, such that remaining viable inoculum can expand into inhibited regions and initiate growth.

In some aspects, the method for affecting the growth topology of aerial mycelium can further include depositing a substrate by hand, screen printing, slurry inkjet printing, or removing substrate in a defined pattern. The method of depositing a substrate can include using a template. The method of depositing a substrate could include depositing the substrate in a defined pattern. The pattern can vary in the exact pattern and spacing across a surface. Non-limiting examples of a pattern can be flat, pyramidal, grooved, or checkerboard.

In some aspects, the method for affecting the growth topology of aerial mycelium can include using different substrates. The method can further include patterning different substrates. The substrates can include for example one or more of the substrates disclosed in the present application.

In some aspects, the method for affecting the growth topology of aerial mycelium can further include depositing nutrients. The method of depositing nutrients can be, for example, by hand, screen printing, or slurry inkjet printing. The method of depositing nutrients can include using a template. The method of depositing nutrients could include depositing nutrients in a defined pattern. The pattern can vary in the exact pattern and spacing across a surface.

In various aspects, a method for affecting the growth topology of aerial mycelium can include using a topology adjustment layer. Non-limiting examples of a topology adjustment layer can include a membrane, a film, a grid, a fabric layer, a sheet, a template, or a layer of substrate, or other configurations suitable for the function described below. A topology adjustment layer can be flexible or stiff. A topology adjustment layer can serve one or more purposes, for example, as a template for positioning spawn, as a tearing structure, to aid separation of aerial mycelium (e.g., a panel) from a solid substrate, as a structural support, as a homogeneous growth support, as a morphology modifier, or as a mechanism for handling or moving the aerial mycelium. Non-limiting examples of the material from which a topology adjustment layer can be made includes metal, fiber (including but not limited to woven fiber such as jute or burlap), a woven or nonwoven sheet, an open-cell porous polymer textile, or a nonwoven flash-spun polymer textile such as Tyvek™, or any material otherwise suitable for such purpose. Nonlimiting examples of a polymer textile can be made of polyethylene, polypropylene, polytetrafluoroethylene, or polyvinylidene fluoride. A porous textile can be for example Porex™ or any material otherwise suitable for such purpose. A porous textile can include pores of sizes between 0.5 microns and 300 microns, between 0.5 microns and 60 microns, between 5 and 250 microns, between 20 and 30 microns, between 100 and 300 microns, or any range between each of these values. A porous textile can include pore volume taking up between 25% and 70%, between 25% and 60%, between 30% and 70%, between 30% and 40%, or any range between each of these values. A nonwoven sheet can be for example spun lace, needle punched, perforated films, or hydro-entangled products, and is not intended to be limiting. A topology adjustment layer can have perforations with various patterns, sizes, or densities. A topology adjustment layer could be designed as, for example, a flat plate, a perforated roller, or a perforated belt.

In some embodiments, the topology adjustment layer can comprise a perforated (e.g., porous) material with a plurality of pores that are relatively small (for example, when the layer is used as a tearing structure), yet configured (e.g., shaped and sized) to allow for one or more hyphal cells of a mycelium to grow therethrough. For example, a hyphal cell may have a width (e.g., diameter) of about 0.5-4 microns. Use of a topology adjustment layer that functions as a tearing structure provides advantages over cutting aerial mycelium from the substrate with a blade, which includes allowing for a clean peeling of the aerial mycelium from the substrate and recovering a larger volume of an intact sheet of aerial mycelium with substantially no substrate. Thus, a topology adjustment layer may be selected which includes at least some amount of pores, or substantially all the pores, with a width (e.g., diameter) that is greater than 0.5 microns, 1 micron, 2 microns, 3 microns, 4 microns, or 5 microns. In some embodiments, a topology adjustment layer can include a plurality of pores with a width (e.g., diameter) that overlaps with the hyphal cell size, but includes a substantial amount of pores that are greater than the hyphal cell size. For example, the topology adjustment layer can include a pore distribution with a pore sized distribution ranging between about 0.5 microns to about 50 microns, with an average of around 3 microns.

In some aspects, a topology adjustment layer can be chemically treated to encourage various growth patterns. For example, growth may be selectively restricted or selectively encouraged in defined locations of the topology adjustment layer by addition of growth inhibitors or additional nutrients. Nonlimiting examples of growth inhibitors include antifungal compounds such as amphotericin, metals such as Cu or Al, or other treatments such as isopropanol or sodium chloride. Nonlimiting examples of additional nutrients include sugars such as glucose, starch, or xylose; nitrogen supplements such as peptone, urea, or specific amino acids; or minerals that may include Mn, Mg, Fe, K, or Cu. A topology adjustment layer may likewise be treated by addition of other compounds such as surfactants like Tween80™ or paramorphogens like sorbose, cellobiose, or lithium. Nonlimiting examples of treatment application include in solid form, as a liquid suspension, sprayed, or printed onto the topology adjustment layer.

The topology adjustment layer can be positioned adjacent to (e.g., directly in contact with) another layer, such as an inoculated or uninoculated substrate. In some aspects, the topology adjustment layer can be extended a selected distance above the growth matrix (e.g., between about 0.1 mm to about 15 cm, or in some aspects about 0.1 to 250 mm, about 1 to 20 mm, or about 5 to 10 mm). The topology adjustment layer can include perforations and be positioned relative to the growth matrix to allow extra-particle growth to grow through the perforations (e.g., in the center of the extra-particle growth). One or more topology adjustment layers may be suspended above the growth surface with spaces as close as 1 mm and as wide as about 15 cm. The one or more topology adjustment layers may be suspended above the growth surface before aerial mycelial growth is initiated or after a time period of growth or may be laid on top of the aerial mycelium after a time period of growth.

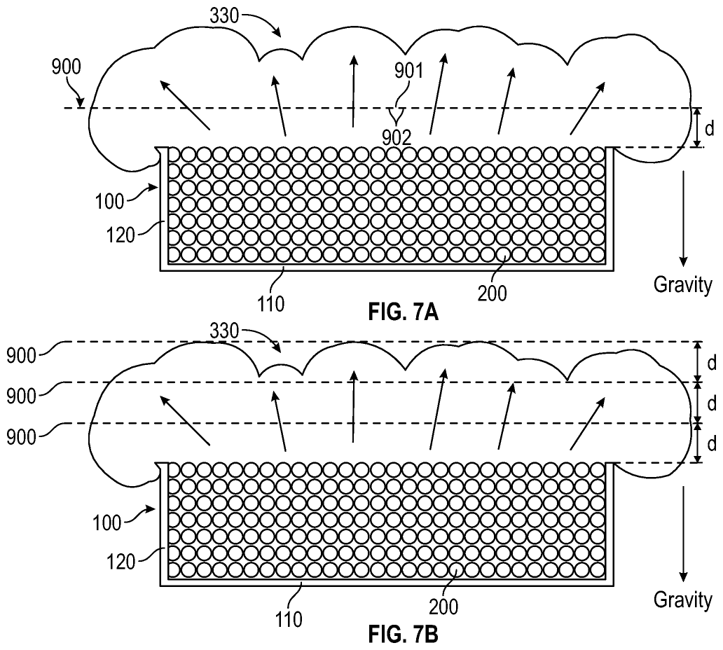

FIGS. 7A and 7B illustrate cross sectional schematic views of two examples of a growth matrix with extra-particle growth extending through one or more topology adjustment layers. The growth unit consists of a single tray container 100 with a bottom 110 and side walls 120. The container 100 contains growth matrix 200 below one (FIG. 7A) or more (FIG. 7B) topology adjustment layers 900 shown as dashed lines. Perforations or other functional openings are illustrated by the blank portions 901 shown between the solid portions 902 of the line 900, which allow extra-particle growth 330 to grow through them. FIG. 7A shows an embodiment with one topology adjustment layer 900 positioned 15 cm (distance “d”) above the growth matrix 200. FIG. 7B shows an embodiment with 3 topology adjustment layers 900 positioned 15 cm apart. The extra-particle growth 330 is the portion shown extending from the growth matrix 200, through perforations or openings, and continuing through to the opposite side of the topology adjustment layer(s) 900. As described above, a topology adjustment layer 900 can be in contact with the growth matrix 200 or can be spaced (e.g., about 15 cm, or in other embodiments about 10 mm) above the growth matrix 200. Some embodiments can include a plurality of stacked (e.g., spaced or directly contacting each other) topology adjustment layers 900 above the growth matrix 200. The topology adjustment layers 900 described herein can be used alone or in combination with other embodiments described herein, such as for example, a perforated layer with a casing layer.

In some aspects, a topology adjustment layer can be a casing layer. A casing layer is a layer of organic or inorganic material placed on top of or below a patterning of spawn. A casing layer can be made of, for example, vermiculite, peat moss, coconut coir, any material that can be used as an uninoculated substrate, or a combination of such materials. A casing layer can be included above or below a patterning of spawn or both above and below a patterning of spawn.

In some aspects, the method for affecting the growth topology of aerial mycelium can include controlling CO₂ level.

In some aspects, the method for affecting the growth topology of aerial mycelium can include controlling mist. In various aspects, mist can be controlled by deposition rate, application, or composition.

In some embodiments, the method for affecting the growth topology of aerial mycelium can include spraying nutrients or inhibitors to particular areas, or using air jets, sound, vibration, direct mist application, or light to modify aerial mycelium topology.

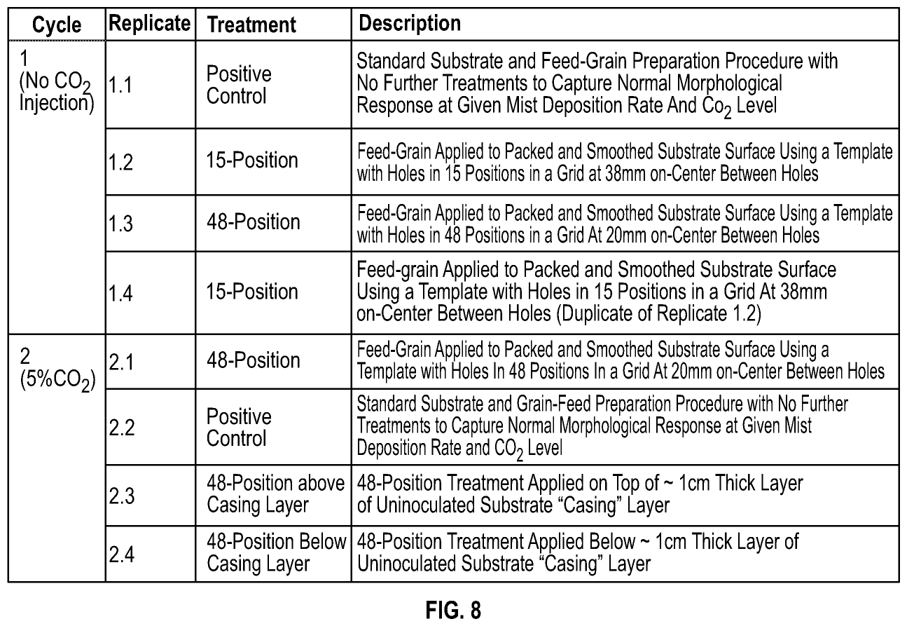

FIGS. 8-29 show experimental support for various embodiments of the present disclosure.

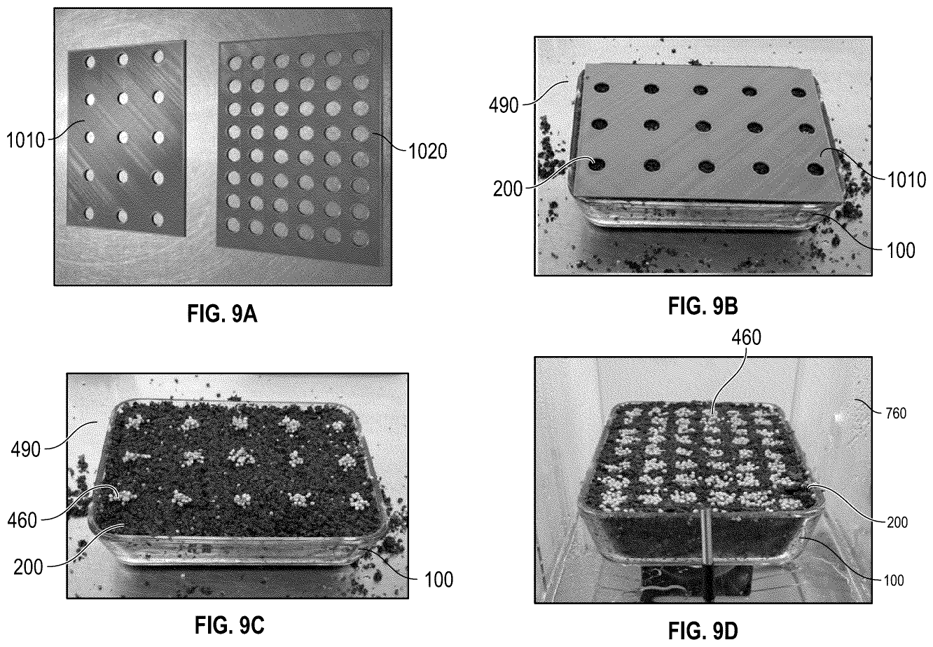

FIG. 8 shows a table of treatments and their descriptions, which correspond to the replicates described in FIGS. 9-29. As used herein, “replicate” is used to refer to a label for an experimental condition. Replicates 1.1, 1.2, 1.3, and 1.4 were grown during a first Cycle without active CO₂ injection with a 5% CO₂ ceiling, resulting in only metabolic CO₂ accumulation, and leading to mean CO₂ levels below 2%. Replicates 2.1, 2.2, 2.3, and 2.4 were grown during a second Cycle with CO₂ injection with a setpoint of 5%, leading to a mean CO₂ level of approximately 5%. All replicates were grown at a temperature setpoint of 85° F. and mist deposition of 0.1-0.3 mg/cm²/hr. All replicates were grown according to the following methods of growing aerial mycelium.

In a first example describing the method of replicates 1.1 and 2.2 used as positive controls, the method of growing aerial mycelium included preparing a substrate and operating a bioreactor. The substrate was prepared in polypropylene mushroom cultivation bags with 50% soy hulls and 50% oak pellets (e.g., Mycomedia™ brand pellets) and water was added to a moisture content of 66-70%. The prepared substrate was sterilized in a pressure sterilizer at 15 psi for 1 hour and was subsequently cooled to room temperature. Once cooled, the prepared and sterilized substrate was aseptically inoculated by the addition of white millet feed-grain particles colonized with Ganoderma sessile strain 045-08-003 (i.e., spawn) at a rate of 22% (calculated as mass of spawn divided by bone-dry mass of prepared and sterilized substrate), creating an inoculated substrate. In some aspects, a fraction of uninoculated prepared sterilized substrate was retained prior to inoculation. The inoculated substrate was loaded into a container at a density of 11.05 lbs/ft³ bone-dry substrate, creating a packed tool with a random distribution of spawn across the substrate surface. The container was, as an example, an 810 mL Pyrex dish of dimensions roughly 45 mm height×180 mm length×130 mm width, which is not intended to be limiting.

The packed tool was placed in a benchtop bioreactor and incubated at 75° F., a relative humidity of 100%, a mist deposition rate from 0.1-0.44 mg/cm²/hr, an airflow velocity from 50-150 ft/min, and a mean CO₂ content of 1.07% (as in replicate 1.1) or 5.06% (as in replicate 2.2) for a period of 7 days. Over the course of incubation, growth progression and morphology was qualitatively observed. At the conclusion of 7 days, samples comprising packed tools with myceliated substrates and resultant aerial mycelium were removed from the bioreactors, scanned with a 3D scanner, and mass and moisture content was collected. Samples were retained for further analysis including microscopy and tensile strength testing. Morphological analyses were performed from 3D scan data.

### Topology Adjustment by Application Spacing

In a second example describing the method of replicates 1.2, 1.3, 1.4, and 2.1, a method of growing aerial mycelium included controlling a pattern of bulbous feature expression across an inoculated substrate surface based on a pattern of spawn application. The process of the first example was followed with the following changes. After loading the container with inoculated substrate, a template was placed over the substrate. For replicates 1.2 and 1.4, the template had 15 12-mm diameter holes in a grid pattern with 38 mm spacing on-center between holes. For replicates 1.3 and 2.1, the template had 48 12-mm diameter holes in a grid pattern with 20 mm spacing on-center between holes. Spawn was spread across the template such that each hole allowed for approximately 10 feed-grain particles, which after removing the template resulted in a grid pattern of spawn application positions across the substrate surface. Incubation and sample processing was further performed according to the first example. In other embodiments, the number of feed-grain particles at each position of application can be, for example, 5-20, or 5-15, or 10-20, or 10-15, or approximately 15, or approximately 1.

### Topology Adjustment by Casing Application

In a third example describing the method of replicate 2.3, a method of growing aerial mycelium included controlling a pattern of bulbous feature expression by stratification of an inoculated substrate, an uninoculated substrate, and a pattern of spawn application. The process of the first example was followed with the following changes. The top ˜1 cm of substrate was a substrate that was not inoculated with spawn and came from the fraction of uninoculated prepared sterilized substrate that was retained in the first example prior to inoculation. The feed-grain application treatment of the second example was then performed, followed by incubation and sample processing according to the first example.

### Topology Adjustment by Alternative Casing Application

In a fourth example describing the method of replicate 2.4, a method of growing aerial mycelium included controlling homogenization of aerial growth by stratification of an inoculated substrate, an uninoculated substrate, and a pattern of spawn application. The process of the first example was followed with the following changes. The top ˜1 cm of the container was left unfilled. Spawn was applied to the substrate surface according to the second example, and an additional ˜1 cm of uninoculated substrate was placed over the spawn application positions such that no spawn was present on the top surface of the substrate (i.e., the substrate-environment interface). Incubation and sample processing was further performed according to the first example.

FIGS. 9A-D illustrate an embodiment of feed-grain application and positions. In FIG. 9A, two feed-grain application templates are shown, the left 1010 with 15 positions at 38 mm spacing and the right 1020 with 48 positions at 20 mm spacing, where each hole has a 12 mm diameter. In FIG. 9B, the positioning of a template 1010 on a prepared tool 490 is shown. In FIG. 9C, the resulting pattern of spawn 460 in 15 positions with approximately 10-15 feed-grain particles per position is shown. The prepared tool 490 comprises a container (or tray) 100 packed with substrate 200 made of a 1:1 soy hull-oak mixture hydrated to a target of 65% moisture content and inoculated at 22% spawn mass/substrate bone-dry mass, packed to a bone-dry mass packing density of 11.05 lb/ft³. For each experimental condition, a template with either 15 positions 1010 or 48 positions 1020 and 12 mm diameter holes was placed over the surface of the prepared tool 490 and spawn 460 was wiped over the template 1010 or 1020 in order to deposit approximately 10-15 feed-grain particles in each position. When the template 1010 or 1020 was lifted from the substrate surface, it left a distinct pattern of feed-grain application. FIG. 9D illustrates a pattern of spawn 460 in 48 positions in a container 100 packed with substrate 200 and placed in a bioreactor 760.

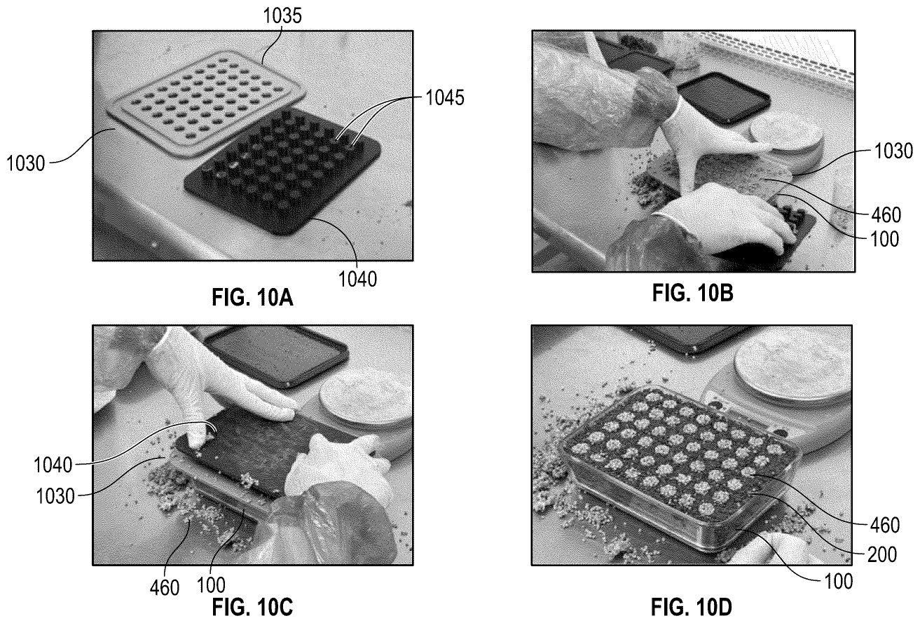

FIGS. 10A-D illustrate an apparatus suitable to implement a method for surface application of feed-grain spawn on a substrate within a container. FIG. 10A shows a feed-grain application template 1030 and stencil punch 1040. The template 1030 has 48 positions at 20 mm spacing, where each hole has 13 mm diameter and 4 mm depth, and a rim 1035, which lines up with the ridge of a matched container 100 to ensure consistent positioning and spacing from the edges of the container 100. The stencil punch 1040 includes 48 round dowels 1045 which line up with the positioning of the holes in the template 1030. The dowels 1045 have 9 mm diameter at 20 mm spacing. The template 1030 is placed over a container 100 which has been loaded with substrate 200, wherein the rim 1035 of the template 1030 is aligned with the ridge of the container 100. Keeping pressure on the template 1030, homogenized spawn 460 is applied over the face of the template 1030 (FIG. 10B). Maintaining pressure on the template 1030, the spawn 460 is worked into the holes of the template 1030 with the flat side of the stencil punch 1040 using a rotating motion, and the excess spawn 460 is removed such that the holes of the template 1030 are clearly visible and filled (not shown). Maintaining the position of the template 1030, the stencil punch 1040 is turned over and positioned to align the dowels 1045 with the holes of the template 1030 (FIG. 10C). Pressure is applied to the stencil punch 1040, and the template 1030 is released from the container 100 (not shown). The template 1030 and stencil punch 1040 are raised away from the container 100 together, trying not to disturb the deposited spawn 460 (not shown). The resulting surface application is shown in FIG. 10D. As an optional additional step, stray spawn deposits may be removed, such as with tweezers. It will be understood that the specific dimensions and number of components and configuration of the embodiments described above with reference to FIGS. 9A-10D (or elsewhere herein) are for illustrative purposes only.

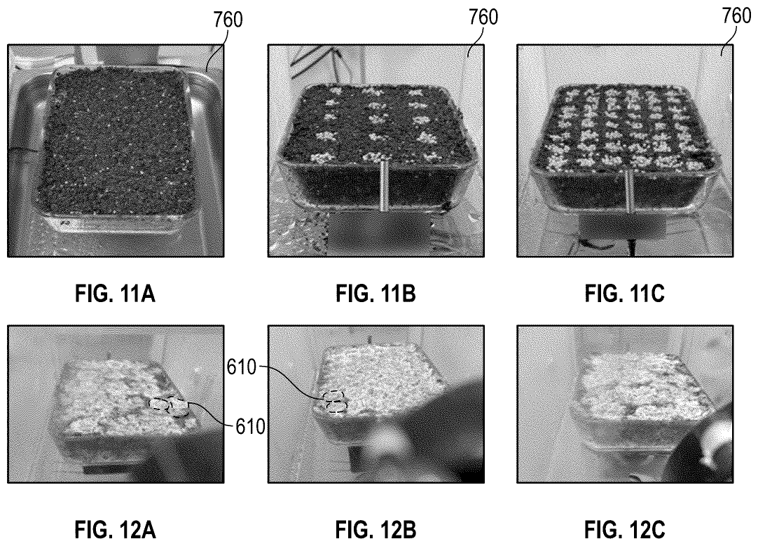

FIGS. 11-21 illustrate a visual timeline of growth morphology for each experimental condition.

FIGS. 11A-C illustrate three replicates (1.1, 1.2, and 1.3) in Cycle 1 placed in the bioreactor chamber 760 at day 0. Cycle 1 was grown with metabolic CO₂ (no injection of CO₂).

FIGS. 12A-C illustrate three replicates (1.2, 1.3, and 1.4) in Cycle 1 at day 2, showing distinct radial colonial expansion 610 extending from each area of feed-grain application and adjacent colonial margins beginning to coincide with one another.

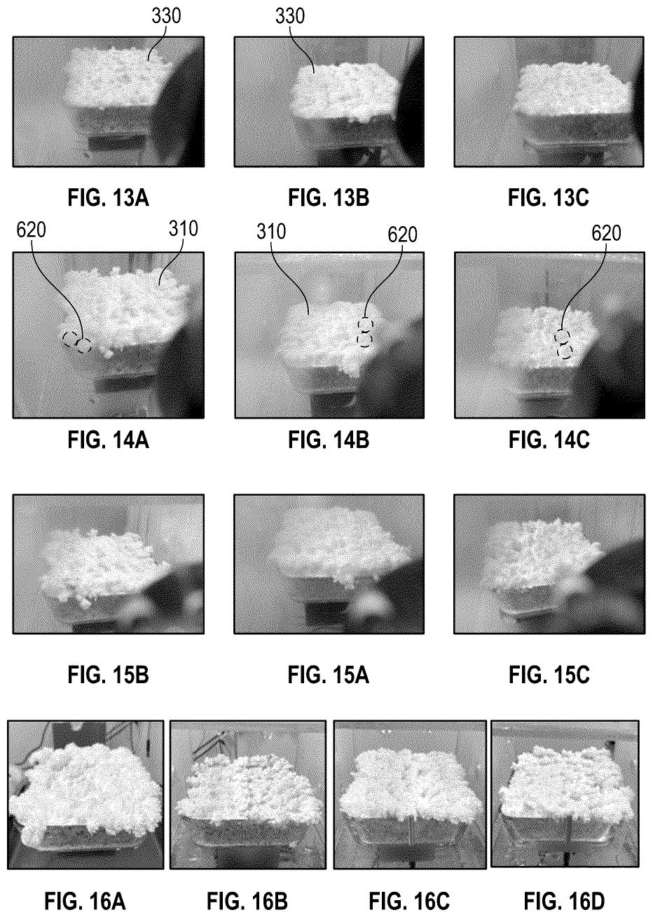

FIGS. 13A-C illustrate three replicates (1.2, 1.3, and 1.4) in Cycle 1 at day 3, showing initiation of aerial growth 330 in all replications. Aerial growth 330 was localized to the regions between feed-grain applications where discrete colonial margins were coinciding in the previous day's observation. This pattern of bulbous initiation between feed-grain applications was more distinct and homogeneous with the 48-position treatment compared to the 15-position treatment. The feed-grain particles of the spawn themselves and substrate regions immediately around them showed resupinate growth with a lack of aerial initiation.

FIGS. 14A-C illustrate three replicates (1.2, 1.3, and 1.4) in Cycle 1 at day 4, showing a general pattern of aerial growth 330 that is evident through the remaining incubation time. All replicates showed bulbous and eccentric discrete aerial growth formations 620 between feed-grain application locations (roughly in positions where discrete colonial margins extending from the feed-grain locations coincided with adjacent colonies at day 2). The 48-position replicate (replicate 1.3) showed more consistent expression of more regular and discrete bulbous forms 620 as compared to the more eccentric 15-position replicates (replicates 1.2 and 1.4).

FIGS. 15A-C illustrate three replicates (1.2, 1.3, and 1.4) in Cycle 1 at day 6, showing growth becoming considerably more coralloid with little further development in terms of panel homogeneity or volume.

FIGS. 16A-D illustrate four replicates (1.1, 1.2, 1.3, and 1.4) in Cycle 1 at day 7 at the conclusion of incubation. All panels shared a distinctly heterogeneous and coralloid morphology, with replicate 1.3 showing more regular expression over the substrate surface.

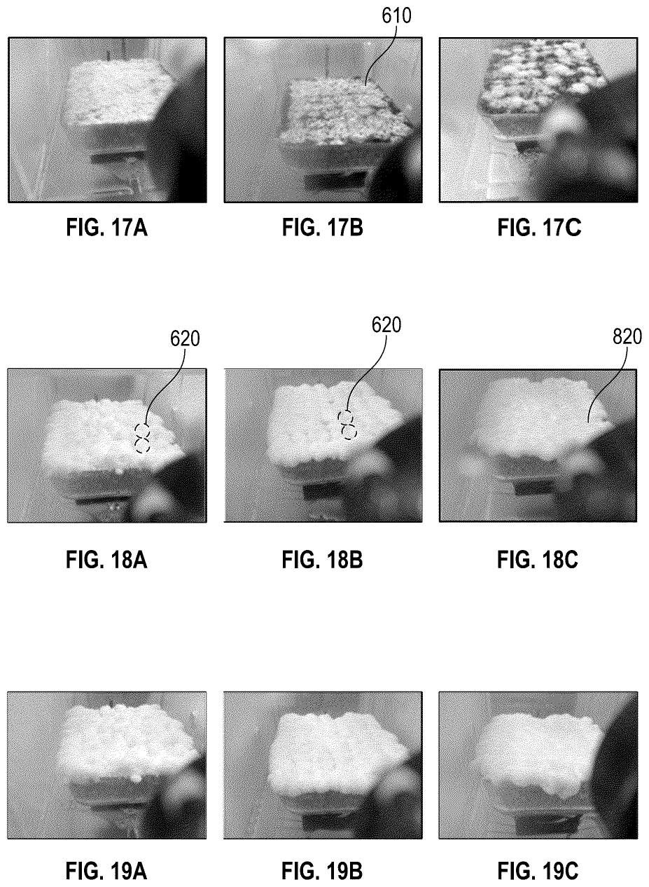

FIGS. 17A-C illustrate three replicates (2.2, 2.3, and 2.4) in Cycle 2 at day 2. Cycle 2 was grown with CO₂ injection at a setpoint of 5%. The appearance of replicate 2.3 (48-position above an uninoculated casing layer) was similar to that observed with replicate 1.3 (48-position in Cycle 1), where discrete colonial margins 610 were converging with one another. The appearance of replicate 2.4 (48-position below an uninoculated casing layer) showed a fairly regular pattern of cottony to floccose colonies without resupinate restricted areas due to spawn being present on the substrate surface.

FIGS. 18A-C illustrate three replicates (2.2, 2.3, and 2.4) in Cycle 2 at day 4. Similar to Cycle 1, the general pattern of mycelium topology that would be retained through the remainder of the cycle was essentially established. The general morphology of replicate 2.2 (Cycle 2 positive control) was similar to replicate 1.1 (Cycle 1 positive control) and consistent with historical samples, wherein discrete bulbous forms 620 are visible. Replicate 2.3 (48-position above a casing layer) showed high geometric regularity of both the size and the positioning of discrete bulbous forms 620. Replicate 2.4 (48-position below a casing layer) showed a meaningful increase in homogeneity of expression resulting in less discrete distinguishable bulbous forms 820 compared to replicates 2.2 or 2.3.

FIGS. 19A-C illustrate three replicates (2.2, 2.3, and 2.4) in Cycle 2 at day 5. There was little difference in growth height, growth volume, and morphology observed between day 4 and day 5, suggesting growth rate was beginning to deflect at a similar timepoint to that observed in Cycle 1. It is hypothesized that nutritional restriction may be a factor here.

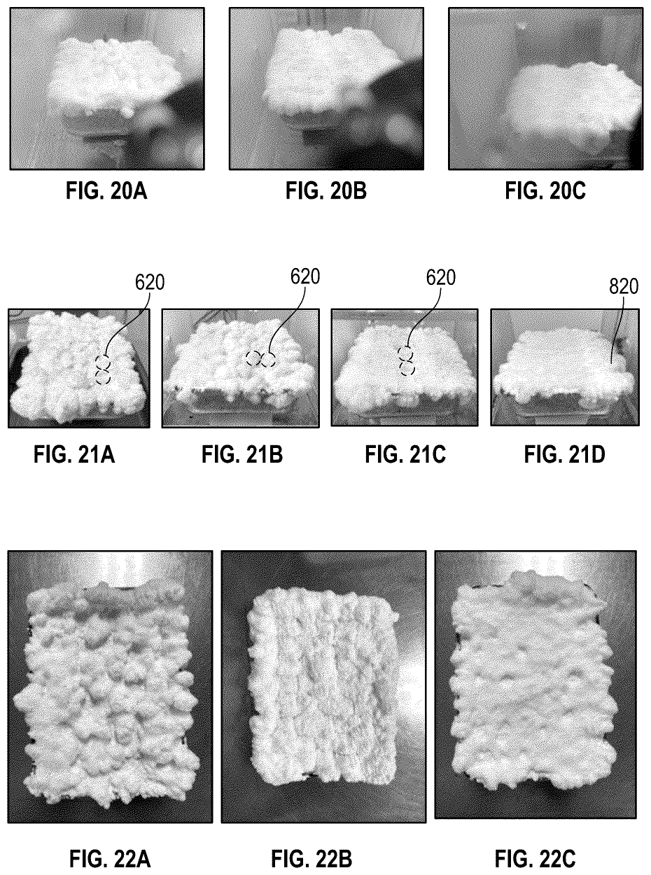

FIGS. 20A-C illustrate three replicates (2.2, 2.3, and 2.4) in Cycle 2 at day 6. Growth height, growth volume, and morphology was essentially unchanged from day 4 to 6 other than a slightly more cottony texture beginning to develop at day 6. Growth appeared to be essentially stalled at this point.

FIGS. 21A-D illustrate four replicates (2.1, 2.2, 2.3, and 2.4) in Cycle 2 at day 7 at the conclusion of incubation. From left to right: Replicate 2.1 (48-position) shows geometric regularity of visible bulbous features 620. Replicate 2.2 (positive control) shows visible bulbous features 620 of inconsistent size, shape, and spacing. Replicate 2.3 (48-position above a casing layer) shows more space-filling bulbous features 620 compared to replicates 2.1 or 2.2. Replicate 2.4 (48-position below a casing layer) shows a lack of distinguishable bulbous forms 820 as compared to any other replicates tested.

FIGS. 22A-C illustrate an overhead view of three replicates (2.2, 2.3, and 2.4) in Cycle 2 at day 7 at the conclusion of incubation, further visualizing the features described in the previous figure.

Referring back to FIG. 4, this drawing illustrates the pattern of growth observed for replicate 2.1. In contrast, application of an uninoculated ˜1 cm casing layer over the spawn application (replicate 2.4) resulted in a homogeneous growth expression of a lack of distinct bulbous features. Furthermore, application of an uninoculated ˜1 cm casing layer before (under) the spawn application (replicate 2.3) resulted in geometric regularity of bulbous features which exhibited more space-filling than those observed in replicates 2.1 or 2.2.

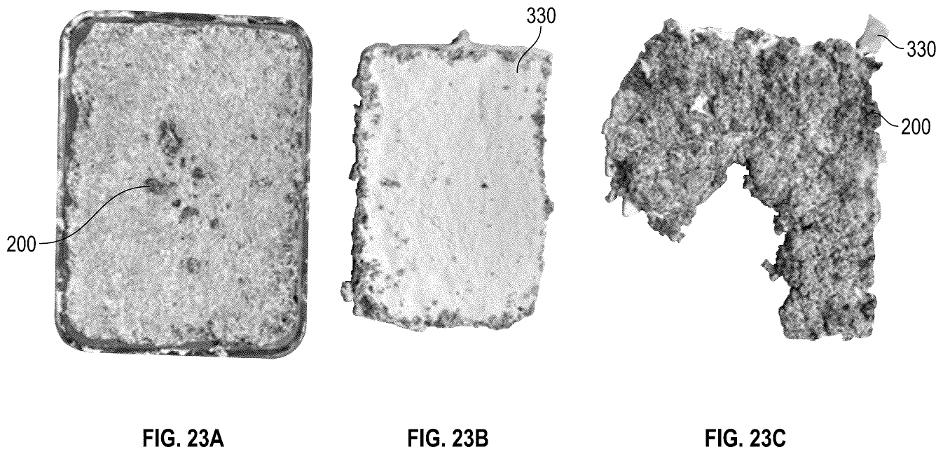

FIGS. 23A-C illustrate intact sheets of aerial mycelium from Cycle 2 at day 7 after extraction, wherein a lack of spawn at the interface of the substrate and aerial mycelium allows for maximum volume recovery of an intact sheet of aerial mycelium with substantially no substrate. FIGS. 23A-B show replicate 2.4 (48-position below a casing layer) peeled cleanly from the uninoculated substrate layer, wherein FIG. 23A shows the substrate 200 remaining in the tool and FIG. 23B shows the underside of the intact sheet of aerial mycelium 330. FIG. 23C shows replicate 2.2 (positive control) peeled from the substrate, wherein the substrate 200 remained heavily bound to the interfacing mycelium 330 as a result of dense growth emanating from the feed-grain particles, resulting in mycelium 330 that did not separate cleanly from the substrate 200. Densification and adhesion to the substrate 200 appeared connected to the presence of feed-grain particles at the substrate-air interface. Replicate 2.4 not only represented the most homogeneous sample, but the lack of feed-grain particles in the substrate surface reduced the overall adhesion between the aerial mycelium 330 and the substrate 200 such that the whole sheet of aerial mycelium 330 could be peeled cleanly from the substrate 200. This allowed for reclamation of aerial mycelium which is typically lost when cutting aerial mycelium from the substrate with a blade and resulted in much higher mass recovery and a more contiguous and consistent separation.

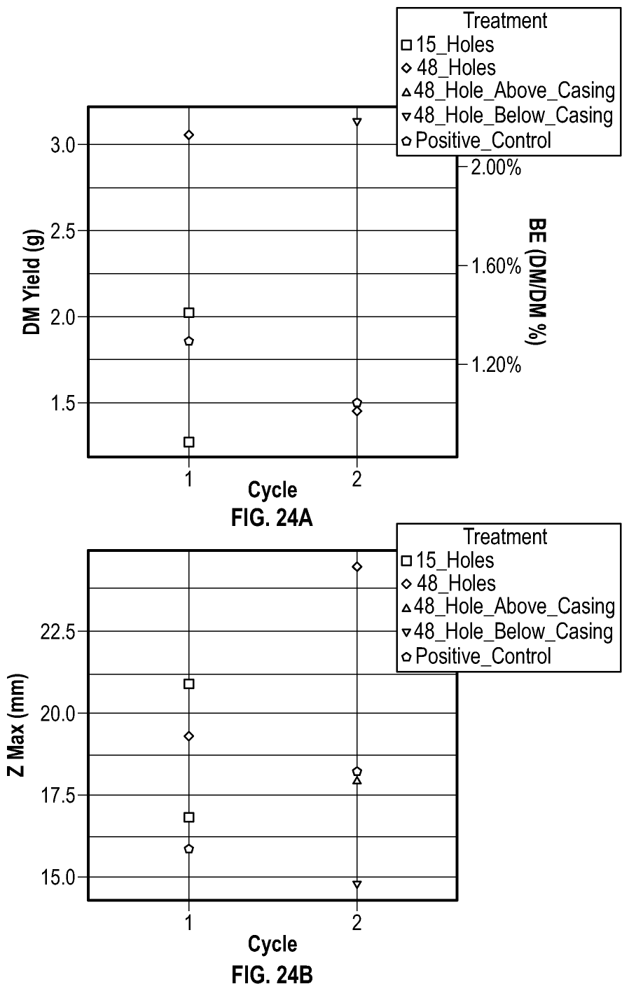

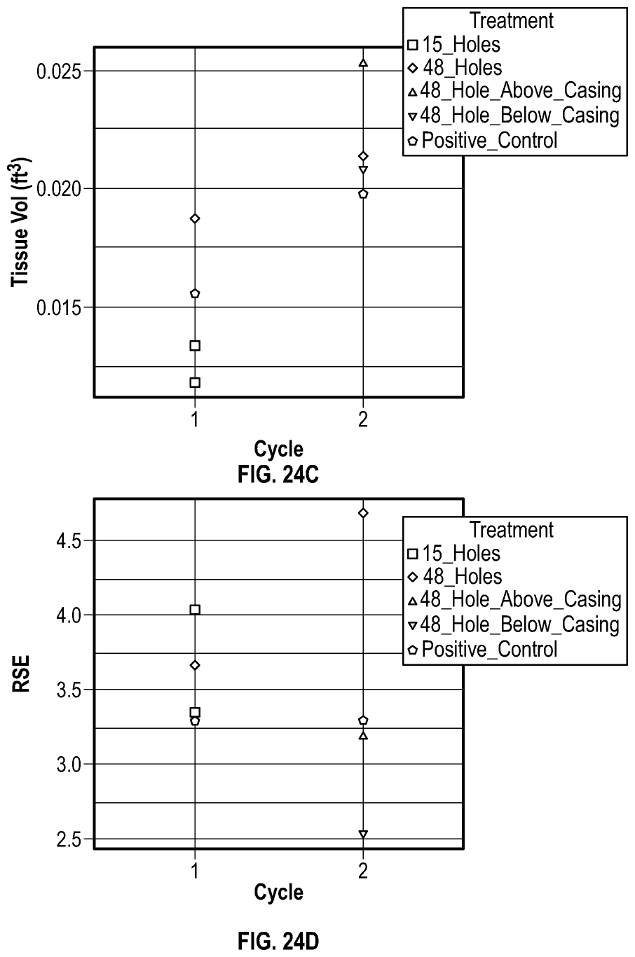

FIGS. 24A-D show graphs comparing physical characteristics of replicates including total bone-dry mass yield (DM Yield; FIG. 24A), growth height (Z max; FIG. 24B), growth volume (Tissue Vol; FIG. 24C), and topological heterogeneity (RSE; FIG. 24D). FIG. 24C shows a meaningful level shift in growth volume between cycles, which may suggest a meaningful effect attributable to elevated CO₂ levels. Replicate 2.4 (48-position below casing) returned both the lowest topological heterogeneity, and therefore the highest homogeneity and geometric topology control, and one of the highest total bone-dry mass yields.

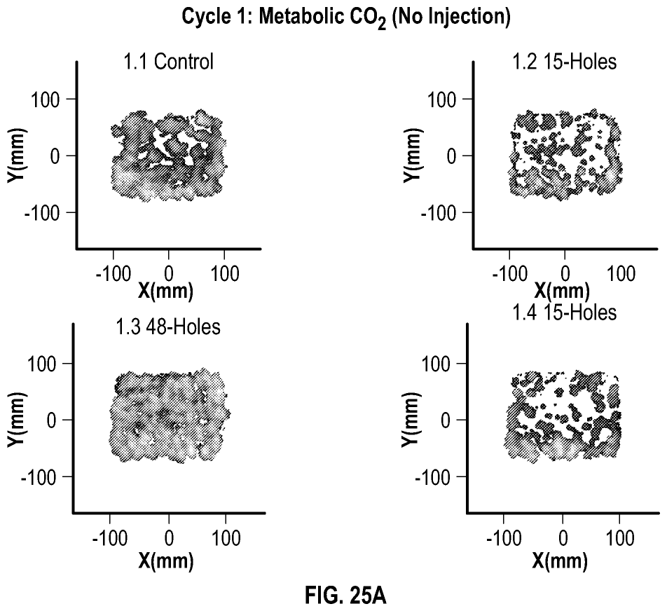

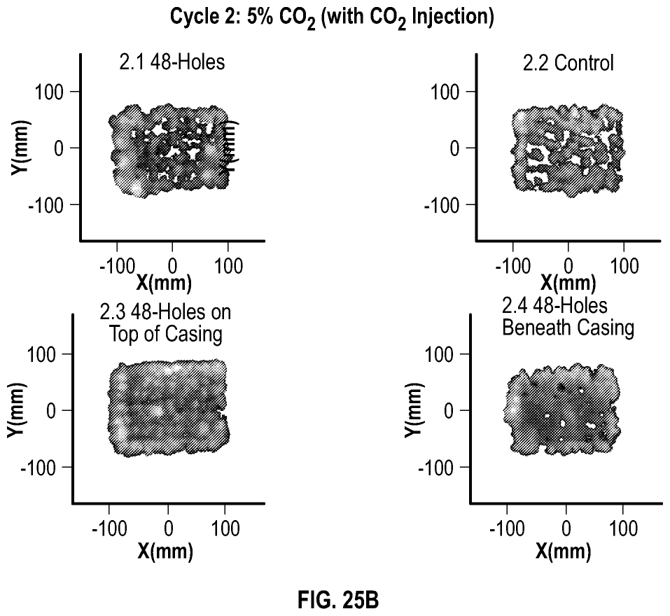

FIGS. 25A-B illustrate luminance height profiles as top-down views of 3D scans of each sample, showing only tissue that grew above the surface of the substrate. Gray value indicates height from the substrate surface, where darker indicates lower height and brighter indicates higher height. These provide a qualitative image of panel growth and can be quantified through image-based morphological analysis.

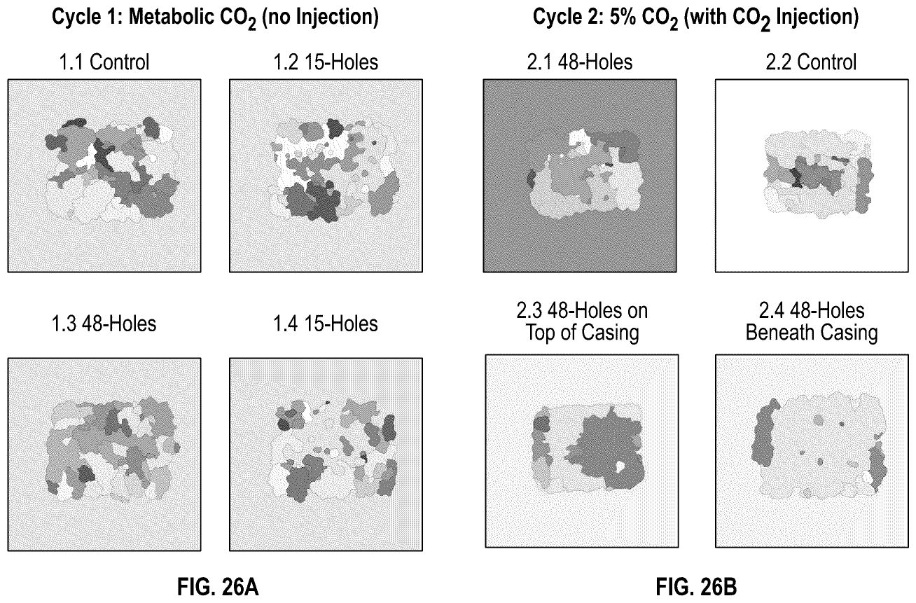

FIGS. 26A-B illustrate morphological segmentation of each of a plurality of point clouds based on their Z by XY luminance height profiles, where the panel is subdivided into the minimum number of discrete morphological features based on the eccentricity of the panel's topology. A more homogeneous panel discretizes to fewer larger segments and a less homogeneous panel discretizes to many smaller segments. This was performed using the ImageJ plugin MorphoLibJ/Morphological Segmentation, although it is hypothesized that other image-based morphological segmentation tools may be implemented, including through ImageJ or FIJI. Replicates 2.3 and 2.4 appear unique in subdividing into fewer larger segments. All replicates of Cycle 2 appear to have fewer larger morphological features than those of Cycle 1. The data shown in FIGS. 26A-B can be used to calculate a segment to area ratio based upon the 36 in² size of the tray in which the panels were formed. For example, the 1.1 control profile includes 39 segments, and thus at least some of the experimental groups demonstrated a segment to area ratio of less than this amount, e.g., less than 1 segment per square inch. The data in FIGS. 26A-B can also define a mean segment area, and a maximum segment area, based upon the 36 in² size of the tray in which the panels were formed. For example, each of the profiles included the following mean segment areas (and maximum segment area): Profile 1.1=648 mm² (max 2626 mm²); Profile 1.2=554 mm² (max 5211 mm²); Profile 1.3=592 mm² (max 3219 mm²); Profile 1.4=406 mm² (max 2482 mm²); Profile 2.1=1152 mm² (max 8087 mm²); Profile 2.2=875 mm² (max 7821 mm²); Profile 2.3=1968 mm² (max 12434 mm²); Profile 2.4=1829 mm² (max 21864 mm²).

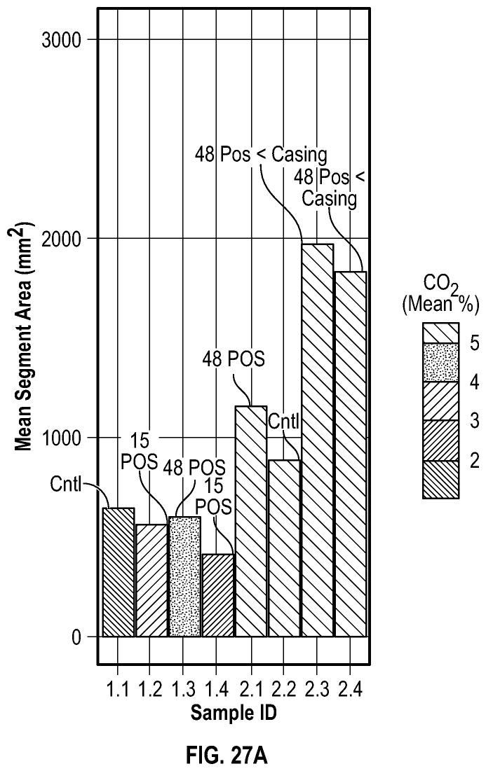

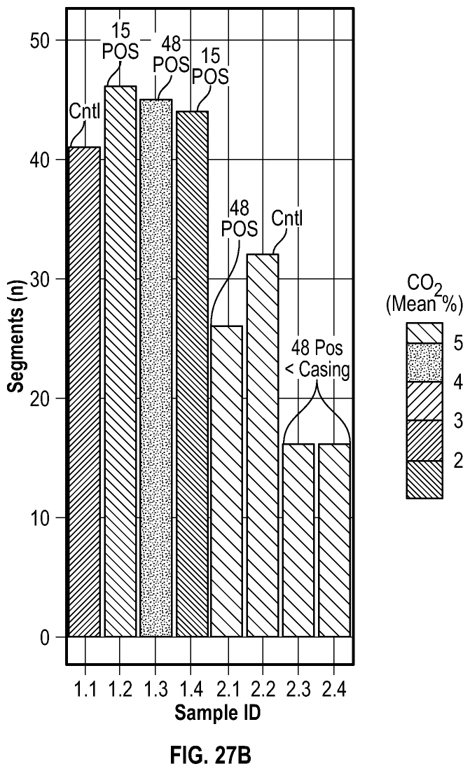

FIGS. 27A-B illustrate mean segment area and number of discrete morphological segments based on image-based morphological analysis and segmentation. Cycle 2 replicates with injected CO₂ appear to produce fewer larger segments (comparing 48-position and control). The replicates incorporating 48 positions with a casing layer produce the largest mean segment size with the fewest discrete morphological features. In general, this suggests the greatest morphological homogeneity is associated with elevated CO₂ combined with the 48-position feed-grain application treatment and application of an uninoculated casing layer.

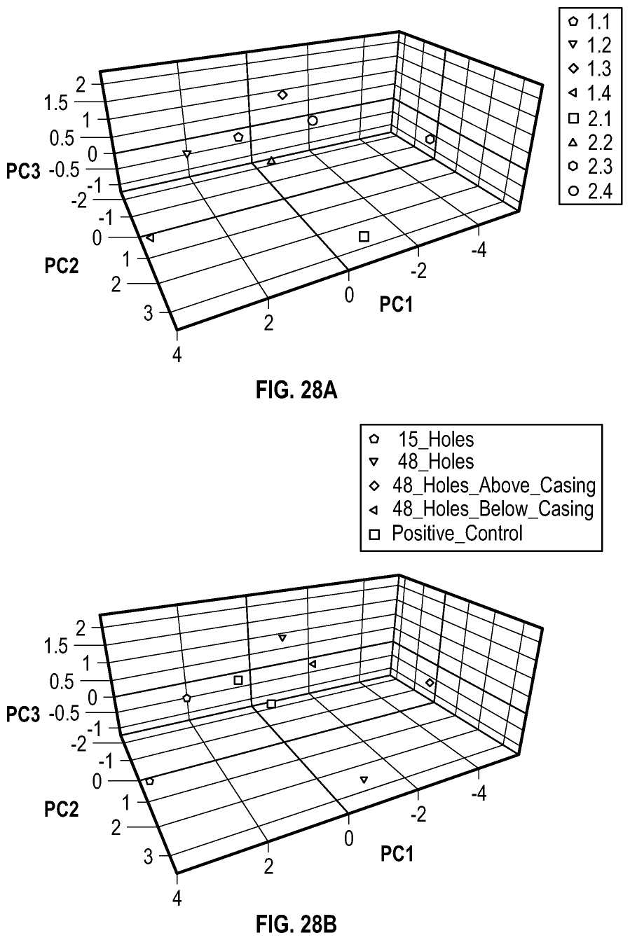

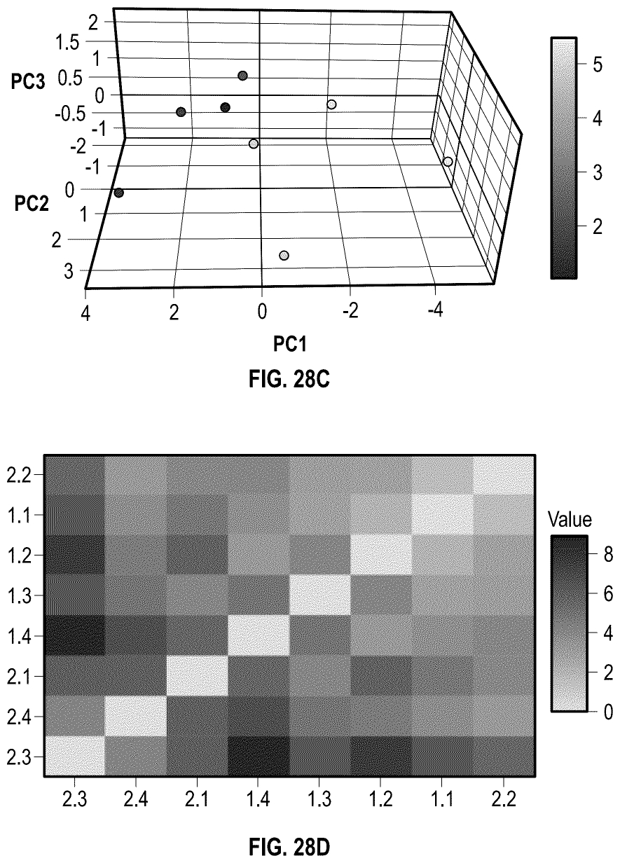

FIGS. 28A-D illustrate relative differences between samples based on total morphological response (FIG. 28A: coding by replicate ID; FIG. 28B: coding by treatment;

FIG. 28C: coding by mean CO₂ %; FIG. 28D: Euclidean distance between samples based on total morphological response). FIGS. 28A-C show clustering between replicates based on the total combined effect of gross physical features and morphological segmentation. FIG. 28D shows Euclidean distance between points within the PCA k-dimensional space, which provides a picture of relative level of difference between samples based on total morphology. There is clear distinction between low and high CO₂ levels, suggesting potentially meaningful morphological consequences to CO₂. There is reasonably good grouping between individual treatments, suggesting independent morphological response spaces between control and 15-position, 48-position applied directly to inoculated substrate, and 48-position applied below or above an uninoculated casing layer. The replicate of 48-position with a casing layer shows some of the furthest distances from control on the Euclidian distance chart, which confirms what can be seen qualitatively.

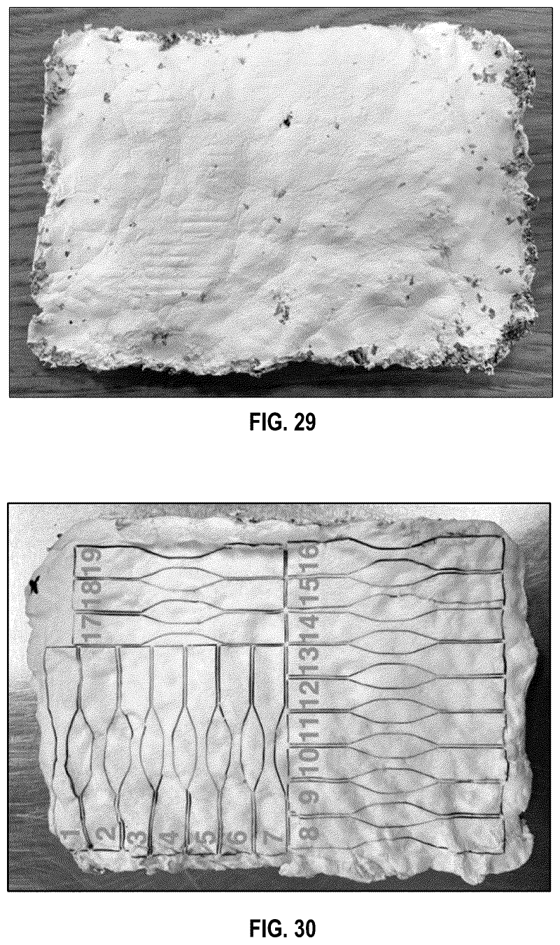

FIG. 29 illustrates replicate 2.4 after completion of the growth cycle, physical removal (peeling) from the substrate, and drying for 24 hours at 110° F. in a commercial dehydrator to a final moisture content of <10%. Once dry, replicate 2.4 consisted of a cohesive sheet of mycelium approximately 16 mm×12 mm with a mean thickness of 2.9 mm.

The sheet from replicate 2.4 was evaluated for tensile strength according to the ASTM D638-10 protocol (incorporated by reference herein), except where deviations were described herein. The ASTM D638-10 protocol provides a standard test method for measuring the tensile properties of plastics and can be used to compare a material's properties (e.g., the tensile strength, the modulus of elasticity, the displacement, and the density) to an international voluntary consensus standard. A laser cutter was used to cut 19 “dog bone” shaped specimens from the sheet according to the layout shown in FIG. 30. Each “dog bone” shaped specimen was tested according to the ASTM D638-10 protocol using an Instron model 3345, resulting in mechanical data for each specimen including a tensile strength, a modulus of elasticity (MOE), a density, and a displacement at tensile strength. Control samples were cut to the dimensions of the sample type IV specification and experimental samples were cut to the dimensions of the sample type V specification. Validation testing was completed on control samples to ensure that analogous results were seen for type IV and V specifications, such that the differences between control samples and experimental samples should not be attributable to differences in specimen dimensions. Test method conditions for the ASTM D638-10 protocol that deviated from type IV to type V also included pre-loading of 0.1 N with a crosshead rate of 10 mm/min and crosshead rate during testing of 50 mm/min.

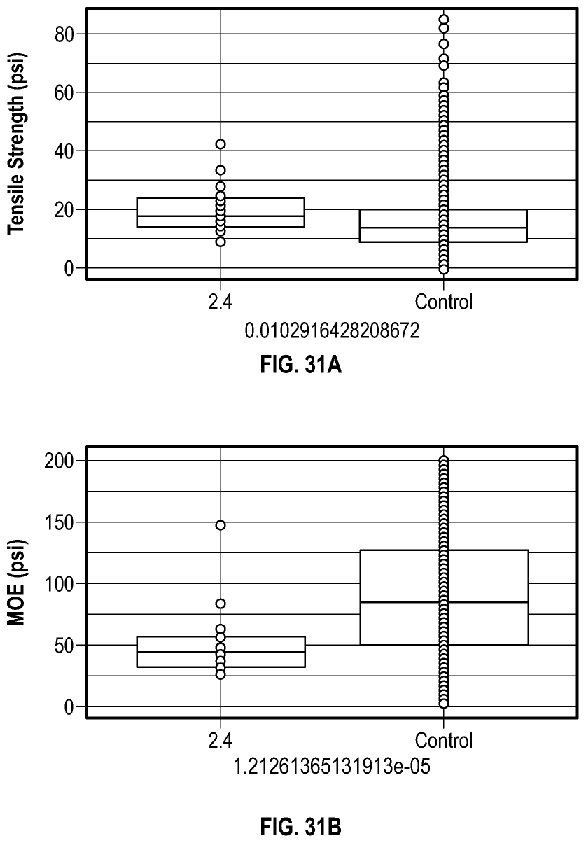

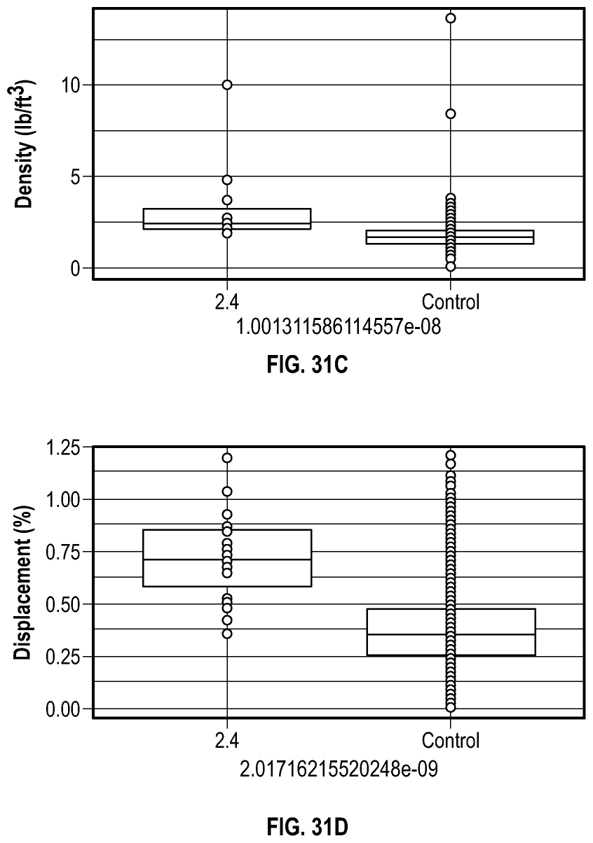

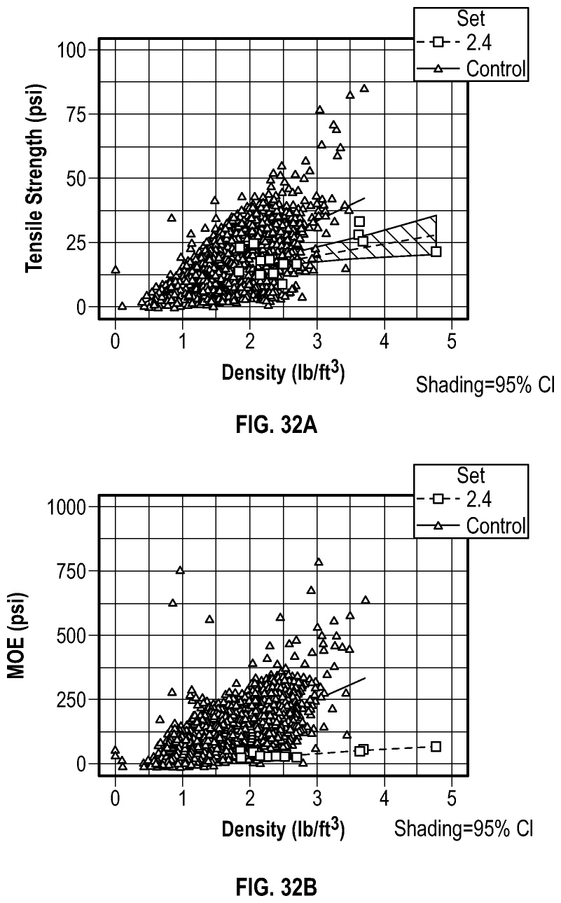

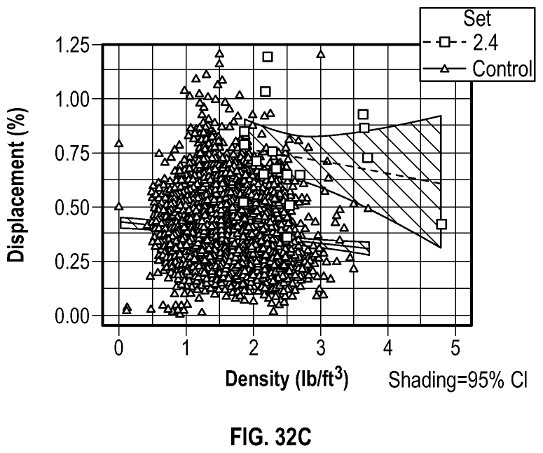

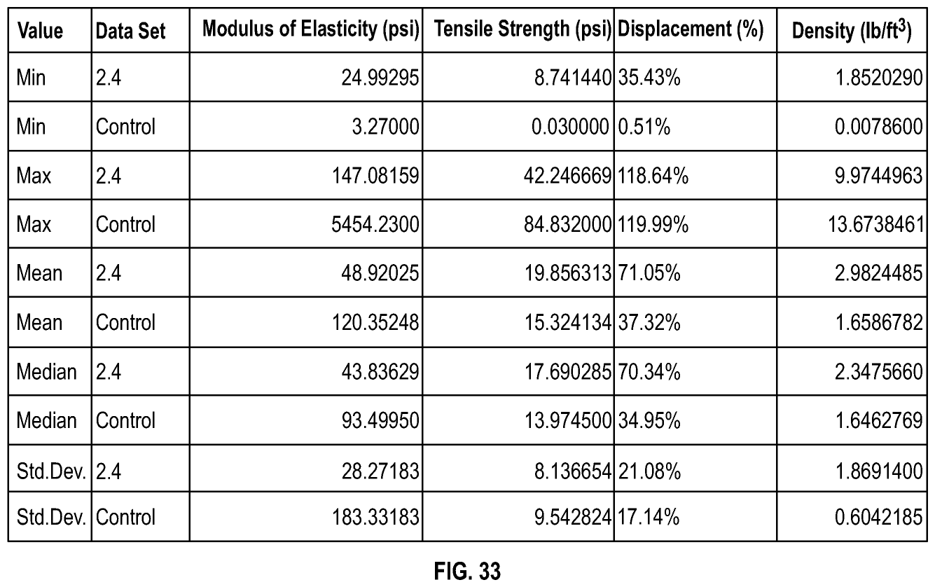

The 19-sample data set produced from the sheet from replicate 2.4 was compared with a population of data collected from aerial mycelium samples produced with the same fungal strain under control conditions, on which tensile testing was similarly performed (data collected between 2016 and 2018, n=2024). FIGS. 31A-D show the resulting mechanical data (FIG. 31A: tensile strength; FIG. 31B: MOE; FIG. 31C: density; FIG. 31D: displacement) for the 19-sample data set from replicate 2.4 (labeled 2.4) and the 2024-sample control condition data set (labeled control). The p-values from non-parametric Kruskal-Wallis one-way analysis of variance are included in the bottom right corner of each plot. The replicate 2.4 group demonstrated highly significant increases in density and displacement (i.e., extension of the sample during testing as a percentage of the gauge length of the “dog bone” sample) as compared to the control group (p<0.001). Additionally, the replicate 2.4 group showed a significantly lower MOE as compared to the control group (p<0.001) and a moderate but significant increase in tensile strength (p=0.01). FIGS. 32A-C show plots of tensile strength (FIG. 32A), MOE (FIG. 32B), and displacement (FIG. 32C) as a function of sample density, wherein the slopes of responses for the replicate 2.4 group are meaningfully different as compared to the slopes for the control group, particularly for MOE and displacement. This demonstrates that the differences observed between the replicate 2.4 group and the control group for these features are not exclusively attributable to density changes. As summarized in the data table of FIG. 33, taken as a whole, the sheet produced under replicate 2.4 conditions demonstrated a unique combination of increased density, increased displacement, decreased modulus of elasticity, and increased tensile strength as compared to the samples produced under control conditions.

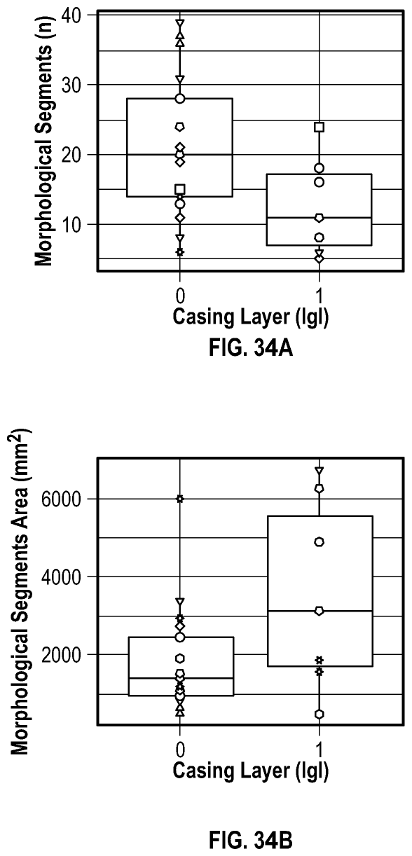

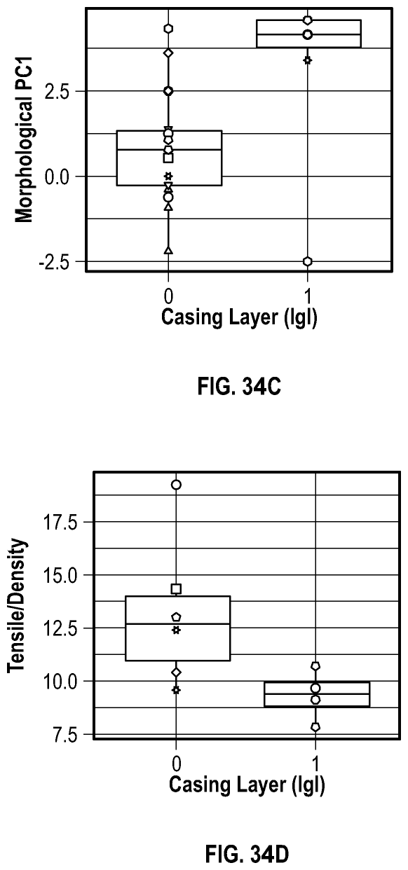

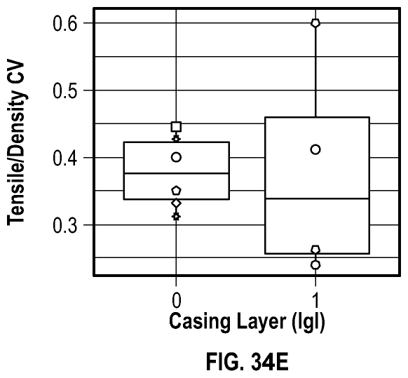

FIGS. 34A-E show morphological and mechanical responses based on application of a casing layer (1 indicates including casing layer, 0 indicates no casing layer). Application of a casing layer is associated with a reduction in the number of discrete morphological features with an increase in mean morphological feature size, as well as an increase in the value of morphological features (e.g., density, bioefficiency, or yield), which generally indicates greater overall morphological homogeneity, higher volume, and reduced directional growth bias. The values shown in the corner of each plot of FIGS. 34A-E indicate p-values for Kruskal-Wallace rank sum test. As seen in FIGS. 34D-E, application of a casing layer is also associated with lower tensile/density values.

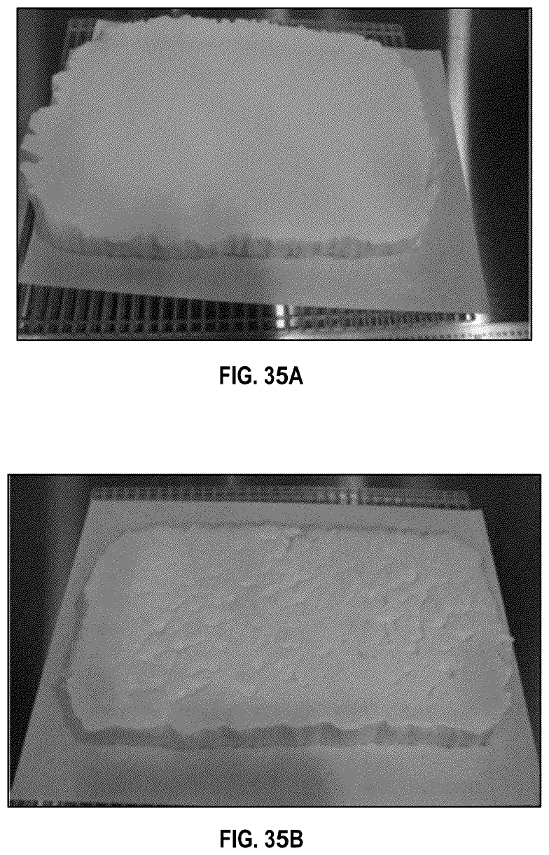

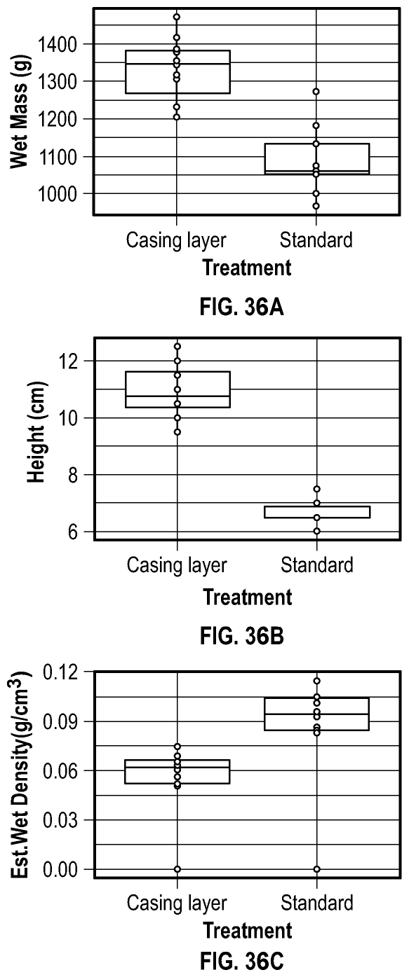

In another example, an observable morphological effect was demonstrated with the use of an uninoculated casing layer. FIGS. 35A-B show a panel cut from a sample grown with an uninoculated casing layer representing 35% of substrate dry mass and a panel cut from a sample grown without a casing layer, respectively. Application of a casing layer qualitatively and meaningfully increased homogeneity. The casing layer treatment also resulted in significant increases in height and mass compared to control, where the high effect was greater than the mass effect, resulting in decreased density. Total harvested wet mass, growth height, and estimated wet density by treatment are shown in FIGS. 36A-C, respectively. Application of a casing layer was associated with an increase in wet mass and height and a decrease in density as compared to control (p<0.001, <0.001 and =0.01 by the analysis of variance (ANOVA), respectively).

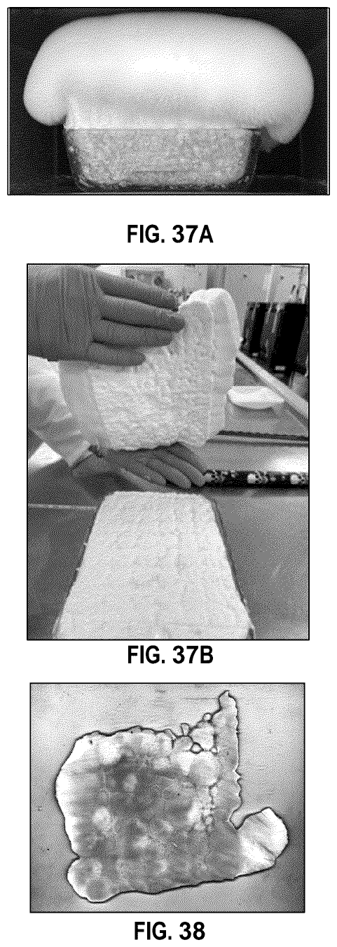

In another example, response to grain application in a defined geometry on top of the substrate surface either below an uninoculated casing layer or without a casing layer was tested. Without a casing layer, a regular pattern of bulbs at the initiation of growth through day 4 was observed, but the level of homogeneity was consistently not maintained after full progression of growth (e.g., by day 7). FIGS. 37A-B show a sample without a casing layer at day 7, wherein the sample demonstrated relatively homogeneous and low-density growth with normal “bulb” distribution reflecting the grain application pattern. In cases where grain application was below a casing layer, initiation of bulb formation was less regularized and did not match the grain application pattern. An example of a mid-section scan from such a sample is shown in FIG. 38. Thus, inoculated grain distribution can regulate bulb initiation. This effect is highly pronounced at early time points when inoculated grain is regularly distributed in discrete points on substrate surface and is diminished or eliminated in later growth time points. The effect is also diminished or eliminated when the inoculated grain is applied below an uninoculated casing layer, even when regularly distributed in discrete points.

### Additives

In some aspects, the present disclosure provides for the incorporation of one or more additives into the mycelial tissue or onto the surface of the mycelial tissue. The additive can be incorporated during or after the growth of the mycelium, and before, during or after any one or more post-processing steps.

In some aspects, an additive can be a colorant. In some embodiments, a colorant is a tannin. An additive can be a naturally occurring additive or an artificial additive, or a combination thereof.

One or more additives can be incorporated into a mycelium of the present disclosure at virtually any step(s) during or between the mycelium growth or post-processing steps described herein.

In some embodiments, one or more additives can be included in (e.g., admixed with) a growth matrix, growth media, growth media substrate, and/or in a further source of nutrition (e.g., a nutritional supplement) in the growth media.

As disclosed in US2020/0024557A1, an additive can be deposited on the growth media during the growth process, either through liquid or solid deposition, or though natural cellular uptake (bioadsorption). Furthermore, during growth, desired additives can be aerosolized into the growth chamber, condense on the propagating tissue, and be incorporated into the growth matrix.

As disclosed herein, an aerial mycelium of the present disclosure can be obtained by depositing aqueous mist onto a growth matrix, an extra-particle mycelial growth or both. The mist can contain a solute, and the solute can be one or more additives. Thus, one or more additives can be incorporated into a growth matrix and/or extra-particle mycelial growth (and thus, into the aerial mycelium obtained therefrom) via misting. These additives may include bioactive elements including bacteria.

As further disclosed in US2020/0024557A1, a mycelial panel can be infused with at least one additive.

In some embodiments, one or more additives is added to a mycelium during the incubation time period. In some embodiments, one or more additives is added to a mycelium after the incubation time period. In some embodiments, one or more additives is added to a mycelium after extraction from the growth matrix.

### Methods Related to Post-Processing Steps

In some aspects, the present disclosure provides for methods of post-processing an aerial mycelium of the present disclosure. Methods of post-processing mycelial tissue, including methods of post-processing a mycelial biopolymer to provide a material that can be used to replace textiles, leather, and leather-like materials, or to provide a high-density foam-like material for use in upholstery, apparel, military gear, athletic gear, and footwear, are disclosed in US 2018/0282529 A1, the entire contents of which are hereby incorporated by reference in their entirety to the extent not inconsistent with the content of this disclosure.

As disclosed herein, an aerial mycelium of the present disclosure can be obtained as a contiguous 3-dimensional object, such as a panel. Thus, an aerial mycelium or a panel or slab thereof can be further characterized by its volume. In some embodiments, the volume of an aerial mycelium (or panel) can be characterized by its thickness, such as its native thickness. In some further embodiments, the aerial mycelial volume can be characterized by its surface area. As such, the surface area of an aerial mycelial (or panel) can be further characterized as having a length and a width.

In some aspects, an aerial mycelium of the present disclosure can be compressed to form a higher density material. The mycelium can be compressed in any direction, such as with the growth-grain or against the growth-grain. In some embodiments, an aerial mycelium can be compressed in a direction substantially parallel with respect to the aerial mycelial growth axis (first axis) to form a compressed aerial mycelium. In some further embodiments, the compressed aerial mycelium has a mean density, wherein the mean density of the compressed aerial mycelium is at least about 2-fold greater than the mean native density of the aerial mycelium.

### Scope of Disclosure

While certain embodiments of the inventions have been described, these embodiments have been presented by way of example only and are not intended to limit the scope of the disclosure. For example, although the present application describes the embodiments of aerial mycelium and processes herein with respect to textiles, it will be understood that the products and processes could be implemented for other applications, such as food. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Although features may be described above as acting in certain combinations, one or more features from a claimed combination can, in some cases, be excised from the combination, and the combination may be claimed as a sub-combination or variation of a sub-combination.

The features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from (i.e., plus or minus) exactly parallel by less than or equal to 45 degrees, 40 degrees, 35 degrees, 30 degrees, 25 degrees, 20 degrees, 15 degrees, 10 degrees, 5 degrees, 3 degrees, 1 degree, or 0.1 degree, and any ranges therebetween. As another example, in certain embodiments, the term “substantially non-parallel” refers to a value, amount, or characteristic that departs from (i.e., plus or minus) exactly zero or 180 degrees by more than 45 degrees, 50 degrees, 55 degrees, 60 degrees, 65 degrees, 70 degrees, 75 degrees, 80 degrees, 85 degrees, and up to 90 degrees, and any ranges therebetween. As another example, in certain embodiments, the terms “generally orthogonal,” “generally perpendicular,” “substantially orthogonal” and “substantially perpendicular” refer to a value, amount, or characteristic that departs from (i.e., plus or minus) exactly 90 degrees by less than or equal to 15 degrees, 10 degrees, 5 degrees, 3 degrees, 1 degree, or 0.1 degree, and any ranges therebetween.

The scope of the present disclosure is not intended to be limited by the specific disclosures of preferred embodiments in this section or elsewhere in this specification and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A method of growing an aerial mycelium biopolymer comprising:
providing an inoculated substrate, the inoculated substrate comprising:
a first substrate; and
a first spawn interspersed throughout the first substrate, the first spawn comprising a second substrate for fungal growth and a first fungus;
providing a second spawn comprising a third substrate for fungal growth and a second fungus; and
positioning the second spawn in a growth pattern approximately parallel to an outer surface of the inoculated substrate, wherein the growth pattern is configured to affect a growth topology of aerial mycelium growing from the first spawn and the second spawn.

2. The method of claim 1, wherein the growth pattern is configured to increase the homogeneity of the growth topology.

3. The method of claim 1, wherein the first spawn comprises a solid fungal inoculum.

4. The method of claim 1, further comprising forming a layer of uninoculated substrate on at least one of the inoculated substrate and the second spawn, wherein the uninoculated substrate comprises a fourth substrate without substantially any fungus.

5. The method of claim 4, wherein forming the layer of uninoculated substrate comprises forming a first layer of uninoculated substrate on the inoculated substrate and forming a second layer of uninoculated substrate on the second spawn.

6. The method of claim 1, wherein positioning comprises positioning the second spawn directly onto the outer surface of the inoculated substrate.

7. The method of claim 1, wherein the method further comprises providing a first topology adjustment layer, wherein the first topology adjustment layer comprises one or more openings.

8. The method of claim 7, wherein positioning further comprises:
positioning the first topology adjustment layer adjacent to the outer surface of the inoculated substrate,
wherein the one or more openings of the first topology adjustment layer form the growth pattern; and
spreading the second spawn across the one or more openings of the first topology adjustment layer.

9. The method of claim 8, wherein positioning further comprises removing the first topology adjustment layer.

10. The method of claim 7, wherein the one or more openings of the first topology adjustment layer comprise a plurality of holes in a grid pattern across the first topology adjustment layer.

11. The method of claim 7, further comprising providing a second topology adjustment layer.

12. The method of claim 7, wherein positioning further comprises positioning the first topology adjustment layer adjacent to the outer surface of the inoculated substrate, wherein one or more of the one or more openings of the first topology adjustment layer comprise perforations, and wherein the first topology adjustment layer is configured to allow for growth through the perforations and further affect the growth topology of aerial mycelium growing from the first spawn and the second spawn.

13. The method of claim 1, further comprising:
placing the inoculated substrate and the second spawn into an incubation chamber;
maintaining the incubation chamber with a predetermined growth environment of humidity, temperature, carbon dioxide content and oxygen content sufficient to produce an aerial mycelium biopolymer consisting essentially of fungal mycelium; and
incubating the inoculated substrate and the second spawn in the incubation chamber for a period of time sufficient to produce the aerial mycelium biopolymer.

14. The method of claim 13, wherein the growth topology of aerial mycelium comprises a vertical growth topology and a horizontal growth topology, and wherein maintaining the incubation chamber comprises maintaining the carbon dioxide content within the incubation chamber at a level between about 0.6% to about 7% to affect the vertical growth topology of aerial mycelium.

15. The method of claim 13, wherein the growth topology of aerial mycelium comprises a vertical growth topology and a horizontal growth topology, and wherein maintaining the incubation chamber comprises maintaining the humidity by introducing aqueous mist into the incubation chamber at a rate of about 0.01 mg/cm²/hr to about 1 mg/cm²/hr to affect the vertical growth topology of aerial mycelium.

16. The method of claim 7, further comprising growing the aerial mycelium through the one or more openings of the first topology adjustment layer.

17. The method of claim 16, further comprising peeling the first topology adjustment layer to separate the aerial mycelium biopolymer from the inoculated substrate.

18. A method of growing an aerial mycelium biopolymer comprising:
providing an inoculated substrate, the inoculated substrate comprising:
a first substrate; and
a spawn interspersed throughout the first substrate, the spawn comprising a second substrate for fungal growth and a first fungus; and
treating the inoculated substrate to prevent growth of the spawn within an inviable portion and permit growth of any remaining spawn within a viable portion, wherein the viable portion forms a growth pattern configured to affect a growth topology of aerial mycelium growing from the inoculated substrate.

19. An aerial mycelium biopolymer grown from the method of claim 18.

20. An aerial mycelium biopolymer comprising a growth surface, wherein the growth surface has a mean native height, and wherein a coefficient of variation of the mean native height is less than 2.

21. The aerial mycelium biopolymer of claim 20, wherein the growth surface comprises a plurality of bulbs forming a preselected pattern.

22. The aerial mycelium biopolymer of claim 21, wherein the plurality of bulbs comprises a plurality of regularly-spaced bulbs.

23. The aerial mycelium biopolymer of claim 21, wherein the growth surface comprises a plurality of depressions forming the preselected pattern.

24.-35. (canceled)
